(19) United States
(12) Patent Application Publication
(10) Pub. No.: US 2022/0081685 A1
(43) Pub. Date: Mar. 17, 2022

(54) CELL-FREE RNA LIBRARY PREPARATIONS

(71) Applicant: Molecular Stethoscope, Inc., San Diego, CA (US)

(72) Inventors: Neeraj Salathia, La Jolla, CA (US); Michael Nerenberg, Del Mar, CA (US); Arkaitz Ibarra, San Diego, CA (US); Jiali Zhuang, San Diego, CA (US)

(21) Appl. No.: 17/243,452

(22) Filed: Apr. 28, 2021

Related U.S. Application Data
(63) Continuation-in-part of application No. PCT/US2019/058961, filed on Oct. 30, 2019.
(60) Provisional application No. 62/752,533, filed on Oct. 30, 2018.

(51) Int. Cl. C12N 15/10

(52) U.S. Cl. CPC C12N 15/1003

(57) ABSTRACT

Diverse cDNA libraries derived from cell-free mRNA and methods of preparing the same are provided. The library may be prepared by extracting RNA from a bodily fluid such as serum or plasma, separating the RNA from contaminants, synthesizing cDNA with a reverse transcriptase enzyme, and enriching protein-coding nucleotide sequences. The library may include a multiplicity of transcripts from solid tissues. Cf-RNA can be measured by qPCR, sequencing, or other suitable methods.

### CROSS REFERENCE

This application is a continuation application of International Application No. PCT/US2019/058961, filed Oct. 30, 2019, which application claims the benefit of U.S. Provisional Application No. 62/752,533, filed Oct. 30, 2018, which is entirely incorporated herein by reference.

### BACKGROUND

A variety of markers are available for detecting various conditions. However, many of these conditions are ones that can affect different tissues. Detecting markers of these conditions in circulation, such as in a blood sample, are not always helpful in identifying which tissue is affected. For example, generic markers for inflammation can indicate an inflammatory response somewhere in the body, but it may not be known which tissue is suffering the response, such as the liver, kidney, lungs, or joints. Tissue-specific tests, such as biopsies, are often invasive, carrying a risk of infection, and typically not comprehensive of the entire organ or tissue. Imaging techniques, such as MRIs and CT-scans, may be used to assess tissue health, but generally can only detect overt features and changes. Thus, these imaging techniques are generally not sensitive enough to pick up early onset of conditions or fairly recent developments of conditions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Cell-free mRNA provides a potential window into the health, phenotype, and developmental programs of a variety of tissues and organs. The present disclosure provides diverse cell-free mRNA libraries enriched in non-blood genes and methods of preparing the same.

In one aspect, provided herein is a method of preparing a cf-RNA sample comprising (a) centrifuging a biological sample at from 1,600 g to 16,000 g and (b) isolating RNA from the biological sample, wherein at least 50, 100, 200, 300, 400, 500, 600, 700, 800, 900, 1000, 1100, 1200, 1300, 1400, or 1500 non-blood genes selected from the list in Table 1 or low stringency non-blood genes selected from Table 10 are present in the cf-RNA sample. The biological sample may be a cell-free biological sample; and may be serum, plasma, saliva, urine, interstitial fluid, cerebrospinal fluid, semen, vaginal fluid, amniotic fluid, tears, synovial fluid, mucus, or lymphatic fluid. In certain embodiments, the biological sample is serum or plasma.

The method of preparing a cf-RNA sample may comprise performing a size selection or immune selection in the biological sample prior to isolating RNA from the biological sample. In some embodiments, performing the size selection comprises centrifugation of the biological sample. Centrifugation may be performed for at least 1 minute, for at least 10 minutes, from 5 minutes to 20 minutes, from 10 minutes to 15 minutes, or for about 10 minutes. In some embodiments, the biological sample is centrifuged at from 10,000 g to 15,000 g. In some embodiments, the biological sample is centrifuged at about 12,000 g. In some embodiments, performing the size selection comprises filtering the sample.

In some embodiments, isolating RNA from the biological sample comprises isolating an extracellular vesicle, which may be an exosome, from the biological sample and isolating the RNA from the extracellular vesicle. In some embodiments, isolating RNA from the biological sample comprises isolating a nucleoprotein complex from the biological sample and isolating the RNA from the nucleoprotein complex.

The method of preparing a cf-RNA sample may further comprising treating the RNA with a deoxyribonuclease. In some aspects, the deoxyribonuclease is TurboDNase I. In some embodiments, the RNA is treated with the deoxyribonuclease in solution.

In some embodiments, isolating RNA from the biological sample comprises contacting the RNA with at least one of an affinity column, a desalting column, or a silica membrane. In additional embodiments, the RNA is contacted with an affinity column, a desalting column, and a silica membrane.

In some embodiments, the method of preparing a cf-RNA sample further comprises enriching at least one protein-coding nucleotide sequence. In other embodiments, the method of preparing a cf-RNA sample comprises depleting ribosomal RNA sequences from the RNA.

In another aspect, provided herein is a method of identifying a cf-RNA molecule comprising (a) isolating RNA from a biological sample; (b) preparing a cDNA library from the RNA; (c) sequencing the cDNA library; and (d) identifying at least one gene in the cDNA library, wherein the biological sample is substantially cell-free, and wherein at least 50, 100, 200, 300, 400, 500, 600, 700, 800, 900, 1000, 1100, 1200, 1300, 1400, or 1500 non-blood genes selected from the list in Table 1 or low stringency non-blood genes selected from Table 10 are detected. In some embodiments, the method of identifying a cf-RNA molecule further comprises aligning sequences from the cDNA library to a reference genome.

In this method of identifying a cf-RNA molecule, in some aspects, the biological sample is cell free. In some embodiments, the biological sample is serum, plasma, saliva, urine, interstitial fluid, cerebrospinal fluid, semen, vaginal fluid, amniotic fluid, tears, synovial fluid, mucus, or lymphatic fluid. In other embodiments, the biological sample is serum or plasma.

In some embodiments, the method of identifying a cf-mRNA molecule identifies at least 1, 5, 10, 20, 50, 100, 200, 300, 400, or 500 tissue-specific genes selected from Table 2; at least 1, 5, 10, 20, 50, 100, or 150 brain-specific genes selected from Table 6; at least 1, 5, 10, 20, or 50 liver-specific genes selected from Table 7 or liver-diagnostic genes from Table 8; or any combination thereof.

In some aspects, the method of identifying a cf-RNA molecule provided herein comprises identifying a first gene, wherein the RNA comprises less than 500, 200, 150, 100, 50, 25, or 15 cf-mRNA polynucleotides that align to the first gene.

In some embodiments of the method of identifying a cf-mRNA molecule, at least 2, 4, 6, 8, or 10 unique fragments are detected per 100 reads. In some embodiments, at least 2, 4, 6, 8, or 10 protein-coding genes are detected per 10,000 reads.

In some embodiments, the method of identifying a cf-mRNA molecule further comprises performing a size selection or immune selection in the biological sample prior to isolating RNA from a biological sample. In some aspects, the size selection comprises centrifugation of the biological sample. The biological sample may be centrifuged at from 1,600 g to 16,000 g; and may be centrifuged for at least 1 minute, for at least 5 minutes, for at least 10 minutes, from 5 minutes to 20 minutes, from 10 minutes to 15 minutes, or for about 10 minutes. In some embodiments, the biological sample is centrifuged at from 10,000 g to 15,000 g, or at about 12,000 g. In other embodiments, performing the size selection comprises filtering the sample.

In some embodiments of the method of identifying a cf-RNA molecule provided herein, isolating RNA from a biological sample comprises isolating an extracellular vesicle from the biological sample and isolating the RNA from the extracellular vesicle. In some embodiments, the extracellular vesicle is an exosome.

In some embodiments, isolating RNA from a biological sample comprises isolating a nucleoprotein complex from the biological sample and isolating the RNA from the nucleoprotein complex. In some embodiments, the method of identifying a cf-RNA molecule further comprises adding an exogenous RNA polynucleotide comprising a first nucleotide sequence to the biological sample and detecting a cDNA polynucleotide comprising the first nucleotide sequence, wherein the first nucleotide sequence of the cDNA polynucleotide comprises a thymine at each position where the first nucleotide sequence of the RNA polynucleotide comprises a uracil.

In some embodiments, the method of identifying a cf-RNA molecule further comprises treating the RNA with a deoxyribonuclease. In some embodiments, the deoxyribonuclease is TurboDNase I. In some embodiments, the RNA is in solution when treated with the deoxyribonuclease.

In some embodiments, the isolating RNA from a biological sample step comprises contacting the RNA with at least one of an affinity column, a desalting column, or a silica membrane. In further embodiments, the RNA is contacted with an affinity column, a desalting column, and a silica membrane.

In some aspects, preparing a cDNA library from the RNA comprising a random sequence, which may be a random hexanucleotide. In some embodiments, the concentration of the random hexanucleotide is at least 60 μM, 70 μM, 80 μM, 90 μM, 100 μM, 150 μM, 200 μM, 300 μM, 400 μM, 500 μM, 600 μM, 700 μM, 800 μM, 900 μM, 1000 μM, 1100 μM, 1200 μM, 1300 μM, 1400 μM, or 1500 μM.

In some embodiments, the preparing a cDNA library from the RNA step of identifying a cf-mRNA molecule comprises forming a single-stranded cDNA. In some aspects, the method further comprising contacting the RNA with a reverse transcriptase to form the single-stranded cDNA. In additional aspects, a double-stranded cDNA is formed from the single-stranded cDNA. In yet further aspects, the single-stranded DNA is contacted with a NEBNext DNA polymerase to form the double-stranded cDNA. In some embodiments, the method further comprises ligating unique dual indexes to both ends of the double-stranded cDNA.

In some embodiments, the method of identifying a cf-RNA molecule further comprises enriching at least one protein-coding nucleotide sequence. The enrichment comprises depleting ribosomal RNA sequences from the RNA in some embodiments, and depleting ribosomal RNA sequences from the cDNA library in some embodiments. In some embodiments, enriching at least one protein-coding nucleotide sequence comprises isolating the at least one protein-coding sequence from the RNA, or from the cDNA. In some embodiments, enriching at least one protein-coding nucleotide sequence comprises hybridizing whole exome baits to the cDNA. The whole exome baits may be RNA polynucleotides or DNA polynucleotides.

Other aspects of the disclosure provided herein are cf-mRNA sequencing libraries comprising cDNA molecules arising from at least 50, 100, 200, 300, 400, 500, 600, 700, 800, 900, or 1000 non-blood genes selected from the list in Table 1 or low stringency non-blood genes selected from Table 10; at least 1, 10, 20, 50, 100, 200, 300, 400, 500, 600, 700, 800, 900, or 1000 non-blood genes selected from the list in Table 1 or low stringency non-blood genes selected from Table 10 per 1,000,000 cDNA polynucleotides; at least 5, 10, 20, 50, 100, 200, 300, 400, or 500 tissue-specific genes selected from Table 2; at least 1, 5, 10, 20, 50, or 100 brain-specific genes selected from Table 6; or at least 1, 5, 10, 20, or 50 liver-specific genes selected from Table 7.

Yet another aspect of the disclosure is a cf-mRNA sequencing library comprising cDNA polynucleotides arising from at least 2000, 3000, 4000, 5000, or 6000 protein coding genes, wherein at least 8%, 15% or 24% of the protein coding genes are non-blood genes.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1 depicts a flowchart of a method of cf-mRNA analysis according to some embodiments of the present disclosure.

FIG. 2A-2E are graphs showing the centrifugation of plasma at forces ranging from 1,900 g to 16,000 g or the filtration of plasma through membranes with pore sizes ranging from 0.2 um to 0.8 um depletes cf-mRNA transcripts derived from red blood cells (FIG. 2A), platelets (FIG. 2B), neutrophils (FIG. 2C), liver (FIG. 2D), and brain (FIG. 2E).

FIGS. 3A-3B are graphs showing enrichment of non-blood cf-mRNA transcripts with increasing centrifugal force. Blood transcripts are depleted at lower speeds than non-blood transcripts. FIG. 3A shows copy number of blood and non-blood transcripts. The number of transcripts was normalized to 1.0 for the 1900 g spin. FIG. 3B shows normalized number of blood and non-blood transcripts per million. For each group, the highest number of transcripts per million was normalized to 1.0.

FIG. 4 is a graph depicting number of non-blood genes detected from cf-mRNA transcripts after centrifugation with forces ranging from 1,900 g to 16,000 g.

FIG. 5 is a graph depicting cf-RNA yields for three RNA extraction kits as determined by qPCR for (3-actin cf-mRNA.

FIGS. 6A-6B are graphs showing enhanced yield of shorter cf-RNA polynucleotide fragments with the QIAamp Circulating Nucleic Acid kit using an optimized miRNA extraction protocol (FIG. 6A) compared with the manufacturer's standard nucleic acid extraction protocol as determined by capillary electrophoresis on a Bioanalyzer (FIG. 6B).

FIG. 7 is a graph depicting enhanced cf-RNA yield with the QIAamp Circulating Nucleic Acid kit using an optimized miRNA extraction protocol compared with the QIAamp ccfDNA/RNA kit.

FIG. 8 is a graph illustrating that treatment of the extracted c-RNA with TurboDNase I in solution eliminated trace contamination with DNA polynucleotides as determined by capillary electrophoresis on a Bioanalyzer.

FIG. 9 is a graph showing that removing inhibitors from the sample increased the apparent yield of cf-RNA as determined by qPCR of 18S rRNA.

FIGS. 10A-10B are graphs showing the OneStep PCR inhibitor removal column (Current desalting column) retains less RNA than other columns the Micro-Bio-Spin column (Desalting column 1) as determined by capillary electrophoresis on a Bioanalyzer.

FIG. 11 is a graph showing reduced cf-RNA extraction failures with the method described in Example 1 (Now) compared with an older method (Before).

FIG. 12 is a graph showing recovery of cf-RNA with the method described in Example 1 is linear, consistent and increases with increased plasma input as determined by qPCR of (3-actin cf-mRNA.

FIG. 13 is a graph quantifying RNA yields with different reverse transcriptase enzymes as determined by qPCR of 18S cDNA.

FIG. 14 is a graph depicting conversion of RNA into cDNA from varied amounts of input cf-RNA as determined by qPCR of 18S cDNA.

FIGS. 15A-15B are graphs depicting unique sequence fragments in cDNA libraries prepared using Accel-NGS 1S Plus (Swift 1) and Accel-NGS 2S Plus (Swift 2) (FIG. 15A) and standard and optimized Accel-NGS 2S Plus protocols (FIG. 15B).

FIGS. 16A-16B are graphs showing misassigned sequences using unique dual indexes (FIG. 16A) compared to standard indexes (FIG. 16B).

FIG. 17 depicts abundance of RNA forms in total cf-RNA (ver1), in rRNA depleted cf-RNA (Ver2) and upon whole exome capture of mRNA (Ver3) as determined by DNA sequencing.

FIGS. 18A-18C are graphs showing the sensitivity of cf-mRNA sequencing using the methods described in Example 1 (Swift 1S) compared to the SMARTer kit with rRNA depletion. Detection sensitivity for ERCC standards (FIG. 18A). Number of genes detected (FIG. 18B). Exemplary determinations of the detection sensitivity for ERCC standards (FIG. 18C). ERCC were standards spiked into samples from four patients (Pt 7171, Pt, 7131, Pt 7139, and Pt 7155).

FIGS. 19A-19E are graphs illustrating comparison of cf-mRNA libraries prepared by the method of Example 1 with cf-mRNA libraries described by Pan et al: (FIG. 19A) number of sequencing reads; (FIG. 19B) number of unique fragments detected; (FIG. 19C) number of protein-coding genes detected; (FIG. 19D) number of genes with >80% coverage; and (FIG. 19E) number of liver genes detected.

### DETAILED DESCRIPTION

Provided herein are methods that can employ upfront centrifugation to reduce contamination of unwanted “blood” transcripts from cf-mRNA sequencing data. The methods herein can reduce background noise arising from blood cell RNA (the “blood component”). Such noise can increase sequencing depth requirements and dilute signal from tissue-specific cf-mRNA.

Protocols, methods and kits disclosed herein can be consistent with a broad range of centrifugal force ranges, such as ranges spanning, lower than or greater than from 1,500 g to 20,000 g, 1,900 g to 16,000 g, 4,000 g to 16,000 g, 8,000 g to 16,000, 10,000 g to 14,000 g, 11,000 g to 13,000 g, 11,500 g to 12,500 g, about 12,000 g, essentially 12,000 g, substantially 12,000 g, or about 12,000 g. Some ranges span about 12,000 g. Some ranges are within 100 g of 12,000 g. Some centrifugation protocols do not differ substantially from 12,000 g, such as centrifugations at 12,000 g. Some ranges are within 100 g of 16,000 g. Some centrifugation protocols do not differ substantially from 16,000 g, such as centrifugations at 16,000 g. Alternate ranges having a starting point at a low figure listed above or ending at a high figure listed above are also contemplated. Such centrifugation protocols can contribute to an improvement such as a 2.5× (for example, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 40 or greater than 4.0×) improvement in the diversity of an extracted cf-RNA sample for processing.

The rate of separation in a suspension of particles by way of gravitational force applied by centrifugation generally depends on the particle size and density. Particles of higher density or larger size generally travel at a faster rate and at some point can be separated from particles less dense or smaller. Alternative technologies for separating particles according to their size include, but are not limited to, gel filtration chromatography and filtration through size-selective membranes. All such technologies are within the scope of this disclosure.

Some commercially available extraction protocols can exhibit high sample extraction failure rates, extract low amounts of cf-mRNA, and fail to eliminate many contaminants that cause downstream assay steps to underperform. Such kits and protocols may only extract sub-populations of either smaller or larger cf-mRNA fragments. As such, provided herein are methods for extracting cf-mRNA from blood, which aids in generating high quality sequencing data that can be rich in biological information. The methods herein may employ a kit for consistent extraction of cf-mRNA from blood with a low failure rate and an enhanced yield of cf-mRNA. Such a yield may retain both the smaller and larger cf-mRNA fragments to produce amplifiable cf-mRNA.

As disclosed herein, some approaches may improve sample extraction success or RNA library diversity through the retention of eluates of at least one extraction wash step, such that small RNA polynucleotides otherwise lost in a wash step eluate are retained so as to contribute to diversity of an RNA library for processing.

Low level DNA contamination can be a source of error in gene-expression quantification, whereas contaminants in blood may inhibit downstream assay biochemistries. Further, commercially available RNA extraction kits can ignore steps to remove DNA or recommend on-column DNAse treatments, which may be suboptimal for robust removal of DNA. For example, in low-yield cf-mRNA samples, low levels of contamination can contribute to significant data misrepresentation. As such, provided herein are methods and systems configured with cf-mRNA washing conditions to remove contaminating substance in blood. Further, such methods may eliminate sporadic genomic DNA contamination of cf-mRNA samples.

Alternately, or in combination, methods and systems disclosed herein may remove contaminating substances by adding an enzymatic DNAse step to remove DNA contamination and/or carry-over. A number of enzymatic and nonenzymatic DNA-removing treatments are consistent with the disclosure herein, often sharing an effect of a removal of DNA from a cf-RNA sample. The methods herein can provide a desalting cleanup column that enhances sample amplifiability (such as by removal of inhibitors) and cf-mRNA enrichment, diversity and yield.

Oligo-dT priming for cDNA synthesis may be suboptimal for fragmented and/or degraded mRNA. In particular, degraded samples may comprise fragments lacking poly-A tails, and incomplete reverse transcription may lead to reverse-transcription products lacking a 5′ region. Accordingly, some systems, methods and kits consistent with the disclosure herein may comprise a step of adding reagents for random priming of reverse transcription, such as using oligos comprising up to 4, 5, 6, 7, 8, 9, 10 or more than 10 bases, such as pentamers, hexamers, heptamers, octamers, nonamers, or decamers. In some embodiments, hexamers may be used to prime reverse transcription.

Further, some commercial enzymes may inhibit cDNA production due to inhibitors from previous steps, whereas quantification of cDNA for reverse-transcriptase enzymes may display poor quantification accuracy when using known RNA inputs.

Provided herein are systems and methods that may improve RNA to cDNA conversion efficiency and quantification accuracy from cf-mRNA. The methods herein may employ relatively high concentrations (such as concentrations greater than those recommended in some commercially available kits) of oligos such as hexamers instead of oligo-dT priming for cDNA synthesis, whereas the best reverse-transcriptase enzyme was selected to produce the highest quantity and amount of cDNA from the RNA inputs. Oligos such as random hexamers or other length oligos can be used at a range of concentrations consistent with the disclosure herein. For example, concentrations of up to, at least, consistent with the range of, about or substantially 60 μM, 70 μM, 80 μM, 90 μM, 100 μM, 150 μM, 200 μM, 300 μM, 400 μM, 500 μM, 600 μM, 700 μM, 800 μM, 900 μM, 1000 μM, 1100 μM, 1200 μM, 1300 μM, 1400 μM, or 1500 μM, or greater than 1500 μM are contemplated. Concentrations within these ranges are also consistent with the disclosure herein, such as at least 60 μM, 70 μM, 80 μM, 90 μM, 100 μM, 150 μM, 200 μM, 300 μM, 400 μM, 500 μM, 600 μM, 700 μM, 800 μM, 900 μM, 1000 μM, 1100 μM, 1200 μM, 1300 μM, 1400 μM, or 1500 μM., or greater than 1500 μM. That is, in some cases, random oligos such as random hexamers can be used at about 200 μM. In some cases, random oligos such as random hexamers can be used at about 500 μM. In some cases, random oligos such as random hexamers can be used at about 1000 μM. In some cases, random oligos such as random hexamers can be used at about 1500 μM. In some cases, random oligos such as random hexamers can be used at about 2000 μM. Fractional concentrations are also contemplated.

Alternately, random oligos such as random hexamers or other length oligos may be used at a higher concentration relative to an amount recommended in a kit. Concentrations such as in a range of 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50× or greater than 50× are contemplated for use with the methods, systems and kits herein. In some cases, concentrations ranging from 15× to 40×, 20× to 35×, 25× to 35×, 28× to 32×, or at least 25×, 26×, 27×, 28×, 29×, 30×, 31×, 32×, 33×, 34×, 35×, or greater than 35× are used, such as, for example, 30×.

The implementation of random hexamers at high quantities and a specific reverse-transcriptase enzyme can enable robust and accurate amounts of cDNA to go into library prep. The methods and systems herein may harness improved cDNA synthesis processes to identify improved Library Prep protocol to reduce the number of sample failures and to improve the richness and robustness of biological data and tissue-specific transcript identification. Such methods can reduce the amount of sequencing resources wasted on uninformative reads, such ribosomal RNA, which can comprise >80% of the transcriptome. As such, the methods and systems herein can include whole-exome enrichment to capture only cf-mRNA. Improvements in assay sensitivity RNA molecule detection can be shown. Further, the methods and systems herein may leverage enrichment protocols which are typically not used for RNA-preps and obtain custom probes to capture spike-in transcript cDNA.

In some embodiments, selected populations of cf-mRNA and/or cDNA derived from cf-mRNA can be enriched by hybridization to baits representative of certain organs or tissues such as brain, liver, lung, bladder, kidney, heart, breast, stomach, intestine, colon, gall bladder, pancreas, lung, prostate, ovary, epithelial, connective, nervous, or muscular. In some embodiments, selected populations of cf-mRNA and/or cDNA derived from cf-mRNA can be enriched by hybridization to baits that distinguish between certain organs or tissues or are diagnostic or prognostic for a disease or condition.

In some instances, methods provided herein can improve the efficiency of converting RNA to a sequence-able cDNA library by, for example, choosing a DNA-seq library kit that exhibits improved efficiency. To facilitate application of a reverse-transcribed cf-RNA kit to a sequencing library protocol, in some cases, cDNA libraries can be treated using a second strand synthesis enzyme or protocol so as to generate a population of double-stranded cDNA molecules representative of the cf-RNA in the sample. Double-stranded DNA molecules so generated can then be subjected to analysis or sequencing library generation using protocols directed to DNA library generation rather than RNA or single-stranded DNA library generation. In some cases, library generation protocols directed to double-stranded DNA are observed to produce higher-quality libraries for downstream analysis such as sequencing libraries, than are produced through protocols directed to library generation from cf-RNA or single-stranded reverse-transcription products. In certain embodiments, cf-RNA can be treated using a method comprising contacting an RNA sample to a reverse-transcriptase such as Superscript IV, prior to a second strand synthesis regimen comprising, for example, an NEBNext polymerase, prior to initiation of a sequencing library protocol directed to double-stranded DNA.

Also provided herein are methods, systems and kits that may mitigate misassignment of sequencing reads to the wrong sample. The methods and systems may minimize loss of cf-mRNA library from stringent cleanup conditions during the enrichment process. Stringent conditions may be required to prevent carry-over of indexed primers that can partake in subsequent PCR amplification of cf-mRNA derived library. The method may comprise employing reagents from IDT technologies with unique dual indexes (UDI) to prevent misalignment of sequencing reeds. When standard indexes were used, sequencing reads were misassigned to a negative control (NTC).

As the majority of transcripts found in blood may be derived from blood-cells, provided herein is a list of “non-blood” genes, which can be detected in blood. The list was determined by merging sample processing (centrifugation speeds) and bioinformatics tools to identify “non-blood” and tissue-specific signatures. Non-blood vs blood transcripts as a function of centrifugation speeds was determined. Centrifugation speeds ranging from 8,000 g to 16,000 g provided a balance between the number of transcripts and genes detected and signal to noise ratio.

A partial list of genes relevant to the identification of non-blood cf-RNA transcripts in blood includes the following: Gene ID SEMA3F; HSPB6; MEOX1; CX3CL1; CDKL3; SEMA3G; DCN; IGF1; WWTR1; PHLDB1; SNAI2; CPS1; RAI14; PREX2; KITLG; ELN; BCAR1; ITIH1; LIMCH1; WISP2; CALCRL; EML1; KIF26A; ACSM2B; ADGRF5; GAL; PTPN21; LMCD1; LNX1; FERMT2; CD5L; NTN4; NUAK1; RASAL2; CTTNBP2; RARB; FBLN1; MAP2; NEBL; HOXA9; RAPGEF3; RIMS1; PTPRH; CADPS2; COL16A1; MECOM; MMP2; PIR; EPB41L1; ARHGAP28; NOS1; FXYD3; RAPGEF4; TF; APOH; PITPNM3; ZFHX4; CCDC80; TGFB2; GABRP; FMO2; CRTAC1; PALMD; PALM; CARD10; RASL10A; RBFOX2; GALNT16; CCM2L; PLS3; ASB9; GABRE; FLT1; ZNF423; NDRG4; CD276; TJP1; PLAT; TUSC3; CLEC4M; NOVA2; SYDE1; RASIP1; ATP6VOA4; CAV1; MET; HOXA5; TSPAN12; SFRP4; MEOX2; RARRES2; GLI3; OGN; LHX6; PTGR1; AMBP; MPDZ; GLIS3; APBA1; ATRNL1; CXCL12; PALD1; CCL2; COL1A1; HLF; KIAA1211; SOD3; CRYAB; APOA4; APOC3; ART4; MGP; CDCA3; AICDA; TPD52L1; LAMA4; C7; FGF1; LIFR; DPYSL3; HRG; AMOTL2; RBP1; FGF12; EVA1A; EFEMP1; IGFBP5; EFHD1; TPO; SDC1; RND3; PARD3B; PRRX1; PRG4; PLA2G4A; NR5A2; ADGRL2; MFAP2; KIF17; HSD11B1; PROX1; APOA1; TTR; ELOVL4; FILIP1; PCDH17; ELOVL3; NKX2-3; TEK; KIAA1217; IQSEC3; TBX2; FABP3; TMEM54; HOXA7; DNAI1; RASSF8; IL13RA2; SLC12A5; PTGIS; POF1B; HIF3A; HIST1H1A; NRN1; SSUH2; MT1G; ID1; F10; RHOJ; AIF1L; MASP1; PTPRB; KDR; RFPL1; A4GALT; KRT17; CPA4; FLNC; MYO1B; CHN1; MYO5C; CGNL1; ISLR; RNASE1; SHC2; DOCK6; APOE; APOC1; USHBP1; UNC13A; PXDN; ASS1; GALNT15; PDLIM4; RAMP2; KHDRBS3; RAI2; NROB2; RHPN2; PPARG; REEP2; HSPA12B; NES; ALDH3B2; BHMT2; STARD13; BEX1; PDZD2; SPINK5; LYVE1; MRO; MEIS2; CABLES1; APLNR; COL4A2; TBX3; AMHR2; HEY2; PKIB; STAB2; THSD1; EDNRB; RAPGEF5; ALPK3; GATA4; DAB2IP; ALDOB; NR5A1; IL33; CCL21; SLCO2B1; LRRC32; SULF1; YAP1; SMAD6; ARHGAP29; TACC2; RBP4; OIT3; AOX1; DUOXA1; GCSH; GATA6; CCDC40; FKBP10; MMEL1; PRDM16; FCN3; TINAGL1; RGS5; RGL1; MALL; RBMS3; IL17RD; SHROOM2; DENND2A; CXorf36; AWAT2; FAM13C; ADIRF; ROM1; OOSP2; CLEC1A; ADGRL3; CCDC102B; DOCK1; MAGI1; THRSP; AKR1C2; PTPN14; HSPB8; TMEM178A; SPARCL1; GJA1; PLOD2; FBXL2; SEMA3D; CABYR; ROBO4; ABI3BP; CEP112; UCHL1; ENAH; PDLIM3; JAM2; FGD5; GNA14; KCNMA1; NMNAT2; CCNB2; AFAP1L1; ERG; HPD; SHROOM4; LAD1; C1QC; CIART; FCN2; AZGP1; COX7A1; CYGB; MPP3; BCL6B; SHANK2; PLPP3; FBLIM1; ADGRL4; SNX7; VCAM1; DDR2; C1orf115; PIGR; RFTN2; FAM84A; NOSTRIN; FABP1; ALB; PRICKLE2; ADAMTS9; APBB2; TM4SF18; EMCN; SPINK1; MYOZ3; BMPER; ZNF704; COL1A2; SOX17; DEFB1; AQP7; KIAA1462; SMCO2; FBN1; LARP6; SPIC; CYYR1; TMEM100; MFAP4; NNMT; GPR182; IGF2; MYO5B; CDC42EP5; SEMA6B; GGT6; KLK4; ACER1; GSDMA; DNASE1L2; ACOX2; FAM107A; COL3A1; FAM178B; CPLX1; EFNA1; SHE; ANTXR1; ROBO1; CTNND2; TM4SF1; MYRIP; FABP4; GPRC5C; GSTA4; PRKCDBP; SOX7; TMEM37; KRT19; PDE7B; KRT20; MAP6; FGA; FGB; PAH; ARNT2; SYNPO2; AGXT; MUCL1; SNTG2; GXYLT2; SNCG; STOX2; C1QTNF1; CD34; PHLDA3; PODN; SLCO2A1; DES; LPL; NR2F1; HOXD8; NUPR1; CIDEA; CLEC14A; C8orf4; CBG; CASKIN2; PTRF; CALML3; PSAPL1; LGALS7B; WSCD1; PIPDX; CDH5; TMEM45A; OR6S1; C1S; BGN; CLEC4G; PYCR1; CTNNA3; FBXL7; FAM167B; MAATS1; DGAT2L6; ALDH1A3; TACSTD2; TCEAL2; WBP5; NR2F2; KRT79; RGS7BP; KRT14; KRTAP23-1; LYPD6; FAM9C; C11orf96; GJA4; NANOS3; PLA2G2A; C15orf52; S100A16; FSIP2; AADACL3; APOD; S100A13; KIF19; HRCT1; ADH1B; CLPSL2; SRGAP1; KIAA1671; FAM177B; HOXA4; MFAP5; PARVA; TEAD4; SULT1C4; ADH4; HMGN5; ZNF442; ARHGEF15; DMD; C1orf53; SMIM9; SOX18; AWAT1; IGFL2; ERICH4; MT1M; C2CD4B; FAM127C; KLHL23; EMP2; UBD; NEURL1B; C1QTNF5; APOC4-APOC2; CFLAR-AS1; PLCL2-AS1; LA16c-395F10.1; C14orf132; AC046143.3; PPP5D1; RP11-14N7.2; HLA-DQB2; PHGR1; RP1-67K17.3; APOC2; RP11-758P17.3; TDGF1; INMT; GSTA1; ETV5; RP11-148B6.1; ECSCR; RP11-548K23.11; IQCJ-SCHIP1; SHANK3; RP11-116G8.5; CTD-2135J3.4; RP11-923111.5; RP11-315D16.2; HOXB7; RP11-521L9.1; RP11-680G10.1; RP11-1260E13.4; GJA5; CTD-2350C19.2; AP000275.65; RP11-45215.2; APOC4; AC003002.4; AC007193.8; DOC2B; CCL14; PIK3R3; RP5-1042K10.14; MATR3; RP11-717K11.2; CDR1-AS; and AL365273.1.

Provided herein are methods that can preferentially deplete blood-cell cf-mRNA and thereby enhance the ability to detect organ derived cf-mRNA, which may be more informative for diagnostic purposes. Centrifugation speed and times may be optimized for each tissue to collect relevant organ-specific transcripts and separate cf-mRNA fractions from different organ-types. Such isolation of “non-blood” organ-specific cf-mRNA from blood can enable extraction of meaningful biological information.

Through an implementation of at least one of the approaches above, up to and including methods, systems and kits employing combinations of the above-mentioned approaches using a majority or all of the approaches herein, one can obtain an improvement, or a substantial improvement, in preparation of cf-RNA library preparation. Such improvement can be observed through at least one of an increase in library diversity such as 2.5× (for example, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 40 or greater than 4.0×) improvement in an RNA library diversity. Increased library diversity can allow the same number of unique genes to be detected with a smaller number of sequencing reads, or more unique transcripts to be observed in the same number of sequencing reads. Similarly, one can observe in various cases an increase, or a substantial increase, in non-blood transcripts sequenced in a cf-mRNA library, such as an increase of up to or at least 10%, 20%, 30%, 40%, 50%, 60%, 70%, or greater than a 70% increase. Some systems, methods, or kits can exhibit an increase, for example, of about 50%. Such increases can be observed prior to or in combination with selective removal of sequences identified as being blood-related transcripts. Such increases can facilitate or can be observed in samples having a reduced volume, reduced sequencing depth or both a reduced volume and sequencing depth relative to some standard protocols. In some cases, sequencing depth can be decreased by up to or at least 10%, 20%, 30%, 40%, 50%, 60%, 70%, or greater than 70% without a corresponding reduction in the number of unique transcripts detected. Some systems, methods, or kits can exhibit a decrease, for example, of about 50%, yet can still provide the improvements in diversity describe above. In some cases, sample volume can be decreased by up to or at least 10%, 20%, 30%, 33%, 40%, 50%, 60%, 70%, or greater than 70%. Some systems, methods, or kits exhibit a decrease, for example, of about 33%, despite the improvements in diversity described above.

In some instances, on an absolute scale, methods, systems and kits consistent with the disclosure herein can increase the resolution of low-abundance transcript sequences in a sequence read library resulting from analysis of a sample as provided herein. As measured by sample transcripts or by internal standard RNA molecules or externally processed molecules assayed concurrently or independently, one can observe inclusion in a final sequence dataset of transcripts present at a low range of molecules per initial sample, such as at least or no more than 900, 800, 700, 600, 500, 400, 300, 200, 100, 90, 80, 70, 60, 50, 40, 30, 20, 15, or 10 molecules per sample. That is, in some cases one can observe inclusion in a final sequence data set of transcripts present at a total amount of, for example, 10-100 molecule per sample. This can represent an improvement over some other methods.

The biological sample for cf-mRNA production may be any biological fluid. Exemplary fluids include blood, saliva, urine, interstitial fluid, cerebrospinal fluid, semen, vaginal fluid, amniotic fluid, tears, synovial fluid, mucus, or lymphatic fluid. Cells may be removed from a biological fluid by centrifugation or other means including filtration. Within the blood, cf-RNA may associate with proteins, lipids, salts, or other components. Some cf-RNA is released from cells in extracellular vesicles such as exosomes. Exosomes may be isolated by methods such as, but not limited to, sedimentation in a centrifuge, size exclusion, filtration, equilibrium density centrifugation, immunoisolation, immunodepletion, and combinations thereof.

In some embodiments, the methods of the present disclosure can allow or permit detection of one or more extracellular RNA transcripts in a biological sample (e.g., a biofluid). The biological sample may be serum, plasma, saliva, urine, interstitial fluid, cerebrospinal fluid, semen, vaginal fluid, amniotic fluid, tears, synovial fluid, mucus, lymphatic fluid, or another suitable biological sample. In various embodiments, the methods can enable detection of one or more cell-free mRNA molecules derived from non-blood cells in a serum sample. The methods can enable detection of one or more cell-free mRNA molecules derived from non-blood cells in a serum sample in addition to hematopoietic transcripts.

The genes detected in cf-mRNA can be traced back to a tissue and/or organ of origin (e.g., tissue-specific genes; see Tables 2-7) or may be of particular interest for diagnosing a disease or condition (see Tables 8-9). Furthermore, the methods provided herein may be sensitive such that extracellular RNA molecules present at a copy number as low as 10, 15, 25, 50, 100, 150, 200 or 500 in the biological sample (e.g., biofluid) may be detected. The RNA molecules can be detected by sequencing, qPCR, ddPCR, microarray, or any other suitable method.

The methods provided herein may detect and/or measure extracellular RNA molecules present in a biological sample (e.g., circulating in a biofluids). In various embodiments, the methods may detect and/or measure cell-free mRNA transcripts derived from hematopoietic and/or non-hematopoietic cells (see, e.g., the non-blood genes of Table 1 or Table 10). The methods may generate purified cf-RNA samples, wherein 1, 5, 10, 20, 50, 100, 200, 300, 400, 500, 600, 700, 800, 900, or 1000 or more non-blood genes from Table 1 or Table 10 can be detected and/or measured. The methods can measure or detect at least 1, 5, 10, 20, 50, 100, 200, 300, 400, or 500 tissue-specific, organ-specific or diagnostically important genes, e.g., from Tables 2-9, from cf-RNA extracted from a biological sample. The methods can generate cf-RNA samples from a biological sample, wherein RNA molecules present at a copy number of at not more than 10, 15, 25, 50, 100, 150, 200 or 500, or less can be detected.

Methods of detecting at least 10, 20, 30, 50, or 100 non-blood cf-mRNAs genes in a biological sample are also provided herein. The methods may include, but are not limited to, (a) centrifuging a serum or plasma sample for at least 10 minutes at from 8,000 g to 16,000 g (or other ranges as provided herein) to form a supernatant; (b) extracting RNA from the supernatant; (c) contacting the RNA with a deoxyribonuclease; (d) forming cDNA from the RNA; (f) preparing a cDNA library from the cDNA; (g) sequencing the cDNA library; and/or (h) aligning the sequences to a reference genome to identify sequences arising from at least 10, 20, 30, 50, or 100 non-blood cf-mRNAs genes per biological sample.

The methods may also include (i) contacting the cDNA library with baits comprising polynucleotide fragments from at least 10, 20, 30, 50, or 100 genes of interest to enrich translated genes. In some cases, the methods (d) may comprise contacting the RNA with a reverse transcriptase enzyme to form a single-stranded cDNA and contacting the single-stranded cDNA with a second strand synthesis enzyme to form double-stranded cDNA. The methods may also include (j) ligating unique dual indexes to the cDNA library to form an indexed cDNA library. In some embodiments, the methods may include (k) pooling up to 2, 3, 4, 5, 6, 7, 8, 9, 10 or more indexed cDNA libraries. The methods may also include (1) performing massively parallel sequencing on the pooled cDNA libraries.

In some embodiments, at least 25, 50, 75, 100, 200, 300, 400, 500, 600, 700, 800, 900, or 1000 genes are detected in the biological sample. In various embodiments, the sequences may be aligned to a reference genome to identify sequences arising from at least 25, 50, 75, 100, 200, 300, 400, 500, 600, 700, 800, 900, or 1000 non-blood cf-mRNAs genes per biological sample. The method may further include contacting the single-stranded cDNA with a second-strand synthesis enzyme to form the double stranded cDNA. In some cases, (c) may be performed in solution.

In certain embodiments, methods of detecting at least 10, 20, 30, 50, or 100 non-blood cf-mRNAs genes in a biological sample may include, but are not limited to, (a) centrifuging or filtering a serum or plasma sample at from 1,900 g to 16,000 g (or other ranges as provided herein); (b) extracting an RNA sample from the supernatant; (c) contacting the RNA sample with a deoxyribonuclease; (d) contacting the RNA with a reverse transcriptase enzyme to form a single-stranded cDNA; (e) forming double-stranded cDNA from the RNA; (f) preparing a cDNA library from the double-stranded cDNA; (g) contacting the indexed cDNA library with baits comprising polynucleotide fragments to enrich translated genes; (h) sequencing the cDNA library; and/or (i) aligning the sequences to a reference genome to identify sequences arising from at least 10, 20, 30, 50, or 100 non-blood cf-mRNAs genes per biological sample.

The methods may further include (j) adding unique dual indexes to the cDNA library to form an indexed cDNA library (e.g., via ligation, PCR, etc.). In some embodiments, the methods may comprise (k) pooling up to ten indexed cDNA libraries. The methods may further comprise (1) performing massively parallel sequencing on the pooled cDNA libraries. In some cases, the methods may further comprise contacting the single-stranded cDNA with a second-strand synthesis enzyme to form the double stranded cDNA. In some embodiments, (c) may be performed in solution.

A polynucleotide sequence that “aligns” to a gene generally has about 100% identity to the sequence of part or all of the gene.

As used herein, the singular forms “a,” “an,” and “the” include plural references unless the context clearly dictates otherwise. Any reference to “or” herein is intended to encompass “and/or” unless otherwise stated.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Methods

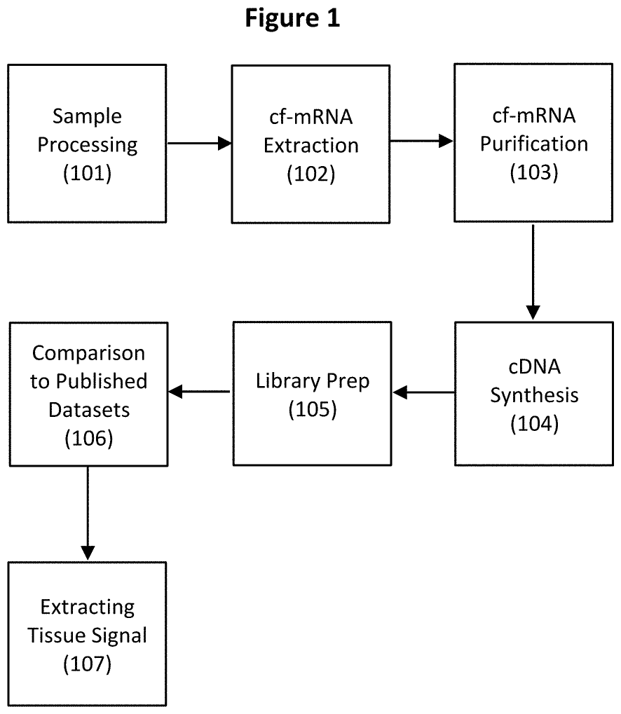

Processes used for cell-free mRNA (cf-mRNA) analysis include biological sample processing, cf-mRNA extraction, cf-mRNA purification, cDNA synthesis, library preparation, DNA sequencing, and bioinformatics (FIG. 1).

Blood was collected in an EDTA vacutainer (BD) for plasma processing or a red-top vacutainer (BD) for serum processing. For serum processing, the blood was incubated for at least 30 minutes at room temperature. After less than 2 hours of post-collection room temperature storage, the blood was centrifuged for 10 minutes at 1600 g to yield plasma or serum (supernatant). Samples can be either processed or frozen for storage at −80 C. To remove residual cells from frozen or fresh samples, the plasma/serum was centrifuged a second time for 10 minutes at 10,000 g to 16,000 g, depending on the application.

Cell-free RNA was extracted and purified using reagents from a QIAamp Circulating Nucleic Acid Kit (Qiagen cat. no. 55114). The following conditions were used for up to 1 ml of plasma or serum. The supernatant was transferred to a new tube, mixed with 130 μl Proteinase K, 1.1 ml Buffer ACL (without carrier), and 330 μl Buffer ATL, and incubated at 60° C. for 45 min. The products were mixed with 3 ml Buffer ACB, 1 μl diluted ERCC RNA Spike-In Mix (Life Technologies cat. no. 4456740), and 2.5 ml chilled isopropanol and incubated for 5 minutes on ice. The sample was loaded onto a QiAmp Mini column using a vacuum manifold, and then washed with 600 μl Buffer ACW1 followed by 750 μl Buffer ACW2, and 2×750 μl EtOH. The column was dried at 56° C. for 10 min. RNA was then eluted twice with 50 μl of Buffer AVE by incubating for 3 minutes each at room temperature followed by 1 minute of centrifugation at 16,000 g. The eluate (˜100 μl) was treated with 3 μl Turbo DNase (Life Technologies cat. no. AM1907) in 1× Turbo DNA buffer for 20 minutes at 37° C. The reaction was stopped with 10 μl DNase Inactivation mix, incubated for 5 minutes at room temperature, and the centrifuged at 10,000 g for 90 seconds.

RNA in the supernatant was brought to 100 μl with water, if necessary, and cleaned using a OneStep PCR Inhibitor Removal Kit (Zymo cat. no. D6030). The Zymo spin column was prepared with 600 μl of Prep buffer, followed by 400 μl and 100 μl of water, all centrifuged at 8000 g for 3 minutes. The sample was then passed through the column by centrifugation at 8000 g for 3 minutes. The sample was then cleaned a second time using reagents from a RNeasy MinElute Cleanup kit (Qiagen cat. no. 74204). The RNA sample was mixed with 350 μl RLT buffer and 900 μl of EtOH and loaded on a RNeasy MinElute column by centrifugation at >8,000 g. The column was washed with 500 μl of RPE Wash Buffer followed by 500 μl of 80% ethanol and then dried as recommended by the manufacturer. For elution, 15 μl of water was added to the column, incubated for 1 min at room temperature, and then collected in a microcentrifuge tube by centrifugation at 16,000 g for 1 minute. For quality control, 1 μl (of 15 μl) was analyzed on a Bioanalyzer using RNA 6000 Pico reagents (Agilent).

cDNA was synthesized using SuperScript IV Reverse Transcriptase (Life Technologies cat. no. 18090050) followed by processing with a NEBNext® Second Strand Synthesis kit (New England BioLabs cat. no. E6111L). RNA (up to 10 μl) was mixed with 1.12 μl random hexamer primers (3 mg/ml) and 0.56 μl dNTPs (10 mM each) in a total volume of 14 incubated for 5 minutes at 65° C., and then chilled to 4° C. The sample was then mixed with 0.43 μl water, 4 μl SSIV Buffer, 0.57 μl DTT (0.1 M), and 1 μl reverse transcriptase (200 U/μl), and incubated at 23° C. for 10 min, 50° C. for 50 min, 80° C. for 10 min, and then held at 4° C. For second strand synthesis, the reaction was supplemented with 4 μl reaction buffer, 2 μl NEBNext Enzyme, brought to a total volume of 40 μl with water, and incubated for 1 hour at 16° C. The dsDNA was cleaned with AMPure XP SPRI beads (Beckman Coulter Inc. cat. no. A63882). 40 μl dsDNA was mixed for 2 minutes with 40 μl Low EDTA TE (Swift Biosciences cat. no. 90296) and 144 μl SPRI beads followed by a 3-minute incubation at room temperature. The beads were collected using a magnetic rack, washed twice with 200 μl 80% ethanol, and air dried for 5 minutes.

A library was prepared with reagents from the Accel-NGS 2S Plus DNA Library Kit (Swift Biosciences cat. no. SP-2014-96) and Unique Dual Indexes (UDI) (Integrated DNA Technologies). The SPRI beads were suspended in 53 μl Low EDTA TE, 6 μl Buffer W1, and 1 μl Enzyme W2 and incubated for 10 minutes and 37° C. 108 μl PEG NaCl Solution (Swift Biosciences cat. no. 90196) was added. The beads were mixed for 2 minutes, incubated for 3 minutes at room temperature, and then collected for 5 minutes on a magnetic rack. After removing the supernatant, the beads were washed twice for 30 seconds with 180 μl 80% ethanol and then air dried. The beads were resuspended in 30 μl Low EDTA TE, 5 μl Buffer G1, 13 μl Reagent G2, 1 μl Enzyme G3, and 1 μl Enzyme G4 and incubated at 20° C. for 20 minutes. 82.5 μl of PEG NaCl Solution was added, followed by 2 minutes of mixing, a 3-minute room temperature incubation, and collection for 5 minutes on a magnetic rack. After removing the supernatant, the beads were washed twice for 30 seconds with 180 μl 80% ethanol and then air dried for 1 minute. The beads were resuspended in 20 μl Low EDTA TE, 5 μl Reagent Y2, 3 μl Buffer Y1, and 2 μl Enzyme Y3 and incubated for 15 minutes at 25° C. 49.5 μl of PEG NaCl Solution was added, followed by 2 minutes of mixing, a 3-minute room temperature incubation, and collection for 5 minutes on a magnetic rack. After removing the supernatant, the beads were washed twice for 30 seconds with 180 μl 80% ethanol and then air dried for 1 minute. The beads were resuspended in 30 μl Low EDTA TE, 5 μl Buffer B1, 2 μl Reagent B2, 9 μl Reagent B3, 1 μl Enzyme B4, 2 μl Enzyme B5, and 1 μl Enzyme B6, incubated at 40° C. for 10 minutes, and then returned to 25° C. 70 μl of PEG NaCl Solution was added, followed by 2 minutes of mixing, a 3-minute room temperature incubation, and collection for 5 minutes on a magnetic rack. After removing the supernatant, the beads were washed twice for 30 seconds with 180 μl 80% ethanol and then air dried for 1 minute. The beads were resuspended in 21 μl low EDTA TE by mixing for 2 minutes followed by a 2-minute incubation. The beads were collected on a magnetic rack, and the supernatant was transferred to a new plate and mixed with 5 μl of Illumina UDI Primer Mix (1-72) (Integrated DNA Technologies), 10 μl Low EDTA TE, 4 μl Reagent R2, 10 μl Buffer R3, and 1 μl Enzyme R4. The PCR reaction was heated to 98° C. for 30 seconds, cycled 16 times at 98° C. for 10 seconds, 60° C. for 30 seconds and 68° C. for 60 seconds, and then held at 4° C. 70 μl SPRI beads were added. Beads and sample were mixed for 2 minutes, incubated for an additional 2 minutes, and collected on a magnetic rack for 5 minutes. After removing the supernatant, the beads were washed twice for 30 seconds with 180 μl 80% ethanol and then air dried for 1 minute. Nucleic acids were eluted in 21 μl water.

cDNA and ERCC DNA were enriched using Sure Select XT V6 whole exome+UTR capture probes and ERCC capture probes in connection with the SureSelect Custom Reagent kit (Agilent Technologies cat. no. 931170) to form cf-mRNA sequencing libraries. Up to ten indexed samples with a total cDNA library mass of 750-1000 ng were pooled. Vacuum centrifugation was used to reduce the volume to 3.4 μl. The sample was then mixed with 5.6 μl SureSelect XT2 Block Mix. 9 μl of the sample was transferred to a PCR strip tube, sealed, incubated at 95° C. for 5 minutes, and then held at 65° C. for at least 5 minutes. 1.5 μl water, 0.5 μl SureSelect RNase Block, 6.63 μl Hyb1, 0.27 μl Hyb2, 2.65 μl Hyb3, 3.45 μl Hyb 4, 1 ERCC capture library (Agilent), and 5 μl Capture Library ≥3 Mb (All Exon V6 60 Mb) were added, and the samples were incubated overnight at 65° C. with a heated lid. MyOne streptavidin beads (50 μl) were prepared by washing four times with 200 μl SureSelect binding buffer. The pooled sample was added to the streptavidin beads and mixed for 30 minutes at 1800 rpm. The beads were collected with a magnet, washed for 15 minutes with 200 μl SureSelect Wash Buffer 1, and then washed three times for 10 minutes at 65° C. with 200 μl SureSelect Wash Buffer 2. Nucleic acids were eluted from the beads by incubating in 20 μl water for 5 min at 95° C., transferred to a new tube, and mixed with 6 μl water, 25 μl 2× Herculase Master Mix, and 1 μl XT2 Primer Mix. The sample was incubated at 98° C. for 2 minutes, cycled 15 times at 98° C. for 30 seconds, 60° C. for 30 seconds, and 72° C. for 1 minute, extended at 72° C. for 10 minutes, and then held at 4° C. The reaction was cleaned with 90 μl AMPureXP beads and eluted in 15 μl water. Products were analyzed by Kapa qPCR and capillary electrophoresis. For Kapa qPCR, dilutions were prepared in 10 mM Tris-HCl, pH 8. Capillary electrophoresis was performed on a Bioanalyzer.

After quantification, sequencing pools were denatured and diluted according to their size and following Illumina's recommendations to obtain optimal clustering. PhiX control was added to the samples as reference. Using a 1000 uL pipette all the diluted library was loaded into reservoir #10 according to the NextSeq 500 (Illumina) instructions. Illumina Basespace was used to conduct sequencing run according to their instructions. Sequencing was conducted with paired end and read cycle was set to 76. NextSeq was selected as the sequencing machine. Sequencing run was started on NextSeq 500 according to the manufacturer's instruction.

Base-calling was performed on BaseSpace platform (Illumina Inc), using the FASTQ Generation Application. For sequencing data analysis, adaptor sequences were removed, and low-quality bases were trimmed using cutadapt (v1.11). Reads shorter than 15 base pairs after trimming were excluded from subsequent analysis. Read sequences greater than 15 base pairs were aligned to the human reference genome GRCh38 using STAR (v2.5.2b) with GENCODE v24 gene models. Duplicated reads were removed using the samtools (v1.3.1) rmdup command.

For cell type deconvolution, a normalization was implemented whereby the expression levels of each gene were divided by its maximum value across the samples. This step rescales expression levels among different genes to avoid domination of the decomposition process by a few highly expressed genes. The normalized expression matrix was then subjected to non-negative matrix factorization (NMF) decomposition using sklearn.decomposition.NMF within the Python library Scikit-learn. NMF decomposition achieves a more parsimonious representation of the data by decomposing an expression matrix into the product of two matrices X=WH; wherein X is the expression matrix with n rows (n samples) and m columns (m genes); W is the coefficient matrix with n rows (n samples) and p columns (p components); and H is the loading matrix with p rows (p components) and m columns (m genes). W is in a sense a summarization of the original matrix H with a reduced number of dimensions. H contains information about how much each gene contributes to the components. Biological interpretation of the derived components was achieved by performing pathway analysis on the top genes that contribute the most to each component.

Whole blood and matched plasma samples were sequenced to identify “non-blood” genes. A gene is considered “non-blood” if its normalized expression (Transcripts Per Million, TPM) is three-fold higher in plasma compared to whole blood (containing blood cells). “non-blood” genes are presumably derived from tissues and/or organs, not blood cells. A blood cell polynucleotide has a sequence that aligns to a blood cell gene and is not a non-blood polynucleotide with a sequence that aligns to a non-blood gene. Non-blood genes represented, on average, 18% of the TPMs in the library, with a range of from 11% to 24%. Non-blood genes represented 15% of all genes detected (gene counted as detected if TPM≥3), range 8%-24%. A list of 2,855 non-blood genes detected in this study is presented in Table 1. A list of lower stringency non-blood genes detected in this study is presented in Table 10.

TABLE 1
Non-blood genes detected in cell-free mRNA

| | | | | | | |
|---|---|---|---|---|---|---|
| A1BG | COL2A1 | GPR88 | MECOM | PROSER2 | SYNPO2L | CTD-2207O23.12 |
| A1CF | COL3A1 | GPRASP2 | MEDAG | PROX1 | SYT17 | CTD-2231E14.8 |
| A2ML1 | COL4A2 | GPRC5A | MEIS1 | PRR15 | SYT5 | CTD-2267D19.3 |
| ABCA3 | COL5A1 | GPRC5B | MEIS2 | PRR16 | TAAR6 | CTD-2270L9.4 |
| ABCA8 | COL6A1 | GPRC5C | MEIS3 | PRR19 | TACC2 | CTD-2302E22.2 |
| ABCC8 | COL6A3 | GPRC5D | MEMO1 | PRR22 | TACR2 | CTD-2313F11.1 |
| ABCG4 | COL8A1 | GPX5 | MEOX1 | PRR7 | TACSTD2 | CTD-2325M2.1 |
| ABHD17A | COLCA2 | GPX6 | MEOX2 | PRR9 | TAGLN3 | CTD-2336O2.1 |
| ABI3BP | COLEC10 | GRAMD2 | MET | PRRG1 | TANC1 | CTD-2369P2.12 |
| ABLIM2 | COLEC11 | GRAPL | METTL1 | PRRG2 | TAS2R38 | CTD-2369P2.4 |
| ABLIM3 | COPZ2 | GRB7 | MEX3A | PRRG3 | TAT | CTD-2396E7.10 |
| ABO | COX5A | GREB1 | MFAP5 | PRRT2 | TBC1D16 | CTD-2396E7.9 |
| AC145212.4 | COX6B2 | GREB1L | MFSD4 | PRRT4 | TBC1D8B | CTD-2517M22.14 |
| ACCSL | COX7A1 | GRM1 | MGAT5B | PRRX1 | TBX1 | CTD-2531D15.5 |
| ACE | CPB1 | GRM2 | MGP | PRRX2 | TBX2 | CTD-2540B15.11 |
| ACE2 | CPE | GRM3 | MID1 | PRSS1 | TBX3 | CTD-2540B15.13 |
| ACER2 | CPEB1 | GRM4 | MIPOL1 | PRSS22 | TBX4 | CTD-2541J13.2 |
| ACOT7 | CPLX1 | GRM8 | MIR205HG | PRSS27 | TCEAL2 | CTD-2545M3.2 |
| ACOX2 | CPNE4 | GSDMC | MIR34AHG | PRSS3 | TCEAL3 | CTD-2561B21.7 |
| ACR | CPNE6 | GSG1 | MISP | PRSS36 | TCF15 | CTD-2561J22.3 |
| ACRBP | CPS1 | GSTA1 | MLF1 | PRSS50 | TCF24 | CTD-2616J11.11 |
| ACSBG1 | CPZ | GSTA2 | MLIP | PRSS54 | TCL6 | CTD-2651B20.1 |
| ACSM2B | CRB1 | GSTM3 | MLNR | PRSS55 | TCP11 | CTD-2666L21.1 |
| ACSM5 | CRCT1 | GSTM5 | MLPH | PRTN3 | TCTEX1D1 | CTD-3035K23.3 |
| ACTG2 | CREB3L1 | GSX1 | MLXIPL | PRX | TDGF1 | CTD-3074O7.5 |
| ACTL7B | CREB3L4 | GTF2A1L | MMACHC | PSAPL1 | TDO2 | CTD-3092A11.2 |
| ACTL8 | CRHBP | GTSF1 | MMEL1 | PSCA | TDRD10 | CTD-3105H18.18 |
| ACTL9 | CRHR1 | GUCY2D | MMP1 | PSD3 | TEAD1 | CTD-3113P16.5 |
| ACVRL1 | CRISPLD1 | GULP1 | MMP2 | PSG1 | TEAD2 | CTD-3137H5.1 |
| ADAD2 | CRNN | GXYLT2 | MMP28 | PSG11 | TEAD4 | CTD-3184A7.4 |
| ADAM18 | CROCC2 | H19 | MMRN2 | PSG3 | TEK | CTD-3193K9.4 |
| ADAM33 | CRP | H1FX-AS1 | MNX1 | PSG4 | TEKT4 | CTD-3214H19.4 |
| ADAMTS17 | CRTAC1 | H2AFY2 | MOBP | PSG6 | TENM4 | CTD-3214H19.6 |
| ADAMTS7 | CRX | HAMP | MOCS1 | PSG7 | TEX13A | EPB41L4A-AS2 |
| ADAMTS9 | CRYAB | HAO2 | MOG | PSG8 | TEX33 | FAM167A-AS1 |
| ADAMTSL1 | CSF3 | HAP1 | MOGAT2 | PSKH2 | TEX35 | FAM171A2 |
| ADCY1 | CSH2 | HAPLN1 | MOV10L1 | PTCHD2 | TEX38 | FOXN3-AS1 |
| ADCY6 | CSPG5 | HAPLN2 | MOXD1 | PTCRA | TF | GATA2-AS1 |
| ADCYAP1 | CSRP2 | HBD | MPDZ | PTGER1 | TFAP2A | GFOD1-AS1 |
| ADGRF5 | CTAGE1 | HBE1 | MPP3 | PTGER3 | TFPI2 | GLYCTK-AS1 |
| ADGRG6 | CTF1 | HCG15 | MPPED2 | PTGES3L | TGFB1I1 | GPR75-ASB3 |
| ADGRL2 | CTGF | HCG25 | MRAS | PTGIS | TH | GRTP1-AS1 |
| ADGRL3 | CTHRC1 | HCN2 | MRGPRD | PTH2R | THBS1 | GS1-114I9.1 |
| ADGRL4 | CTNNA3 | HCN3 | MRO | PTHLH | THEM5 | GS1-114I9.3 |
| ADH1A | CTNNBIP1 | HEIH | MROH8 | PTK7 | THNSL2 | GS1-124K5.4 |
| ADH1B | CTRB2 | HELT | MRPL12 | PTMA | THRSP | HIST1H2AA |
| ADH1C | CTTN | HEPN1 | MRPL23 | PTPN11 | THSD1 | HIST1H2AB |
| ADH4 | CX3CL1 | HES1 | MSANTD3 | PTPN14 | THSD4 | HIST1H2AH |
| ADIRF | CXCL11 | HES6 | MSRB3 | PTPN20 | THY1 | HIST1H2AJ |
| ADM5 | CXCL12 | HFE2 | MST1R | PTPN21 | TICRR | HIST1H2AL |
| ADPRHL1 | CXCL13 | HGD | MSX1 | PTPN5 | TIGD7 | HIST1H2BB |
| ADRA1A | CXCL14 | HHATL | MSX2 | PTPRB | TIMP3 | HIST1H2BH |
| ADRA1D | CXCL3 | HHIPL1 | MT1E | PTPRD | TIMP4 | HIST1H2BK |
| ADRA2A | CXCL9 | HHLA3 | MT1G | PTPRF | TINAGL1 | HIST1H2BM |
| ADRA2B | CXorf36 | HIF3A | MT1M | PTPRG | TJP1 | HLA-DQB2 |
| ADRA2C | CYGB | HIGD2A | MT3 | PTPRH | TKTL2 | HORMAD2-AS1 |
| ADRB1 | CYP11A1 | HIST1H2AI | MTCL1 | PTPRN | TLE6 | HOXA11-AS |
| ADRB3 | CYP19A1 | HIST1H3B | MTNRIB | PTPRR | TLL1 | HSPB2-C11orf52 |
| ADSSL1 | CYP1A1 | HIST1H3C | MTSS1L | PTPRZ1 | TLL2 | IGKV3D-20 |
| AFAP1L1 | CYP21A2 | HIST1H3E | MTUS1 | PTRF | TLX1NB | INO80B-WBP1 |
| AFAP1L2 | CYP24A1 | HIST1H3F | MUC1 | PTRH1 | TLX2 | IQCJ-SCHIP1 |
| AGBL4 | CYP26B1 | HIST1H3I | MUC12 | PURG | TM4SF1 | KCNIP2-AS1 |
| AGXT | CYP27B1 | HIST1H3J | MUC17 | PVT1 | TM4SF18 | KCTD21-AS1 |
| AHRR | CYP2A6 | HIST1H4B | MUC19 | PXDN | TMA7 | KRTAP10-11 |
| AHSG | CYP2B6 | HIST1H4D | MUC3A | PXDNL | TMC4 | KRTAP10-5 |
| AICDA | CYP2C8 | HIST1H4F | MUC4 | PYCR1 | TMEFF2 | KRTAP10-9 |
| AIF1L | CYP2E1 | HIST1H4J | MUC7 | PYCRL | TMEM100 | KRTAP13-3 |
| AIPL1 | CYP3A4 | HIST1H4L | MXRA7 | RAB13 | TMEM108 | KRTAP19-1 |
| AJUBA | CYP3A7 | HIST2H3D | MYBPHL | RAB17 | TMEM119 | LA16C-306E5.3 |
| AKAP2 | CYP46A1 | HJURP | MYCN | RAB30-AS1 | TMEM130 | LA16C-358B7.4 |
| AKR1C1 | CYP4F8 | HLF | MYEOV | RAB3A | TMEM136 | LAMTOR5-AS1 |
| AKR1C2 | CYP8B1 | HMCN1 | MYH7B | RAB3C | TMEM17 | LINC00152 |
| AKR1C3 | CYR61 | HMGA2 | MYL2 | RAB42 | TMEM179 | LINC00504 |
| AKR1D1 | CYS1 | HMGCS2 | MYL6B | RAD51 | TMEM215 | LINC00649 |
| ALB | CYYR1 | HMP19 | MYLK3 | RAD54L | TMEM230 | LINC00657 |
| ALDH1A3 | DAB2IP | HNF1A | MYLPF | RADIL | TMEM231 | LINC00894 |
| ALDH1L1 | DACT2 | HNRNPCL2 | MYO10 | RAET1E | TMEM238 | LINC01011 |
| ALDH3A1 | DAND5 | HOMER3 | MYO15A | RAET1G | TMEM239 | LINC01067 |
| ALDH7A1 | DAO | HOXA10 | MYO18B | RAG2 | TMEM37 | LINC01140 |
| ALDOB | DARS-AS1 | HOXA3 | MYO1C | RAI14 | TMEM40 | LINC01249 |
| ALOXE3 | DBNDD1 | HOXA4 | MYO5B | RAI2 | TMEM44 | LINC01554 |
| ALPK2 | DCANP1 | HOXA5 | MYO5C | RALYL | TMEM52 | LLNLR-245B6.1 |
| ALPK3 | DCHS2 | HOXA7 | MYOZ3 | RAMP2 | TMEM52B | LLNLR-284B4.2 |
| AMBP | DCLK1 | HOXB1 | MYRIP | RAMP3 | TMEM54 | MAGI2-AS3 |
| AMHR2 | DCN | HOXB4 | MYT1L | RANBP1 | TMIE | MAN1B1-AS1 |
| AMN | DCSTAMP | HOXB6 | MYZAP | RAPGEF5 | TMPRSS13 | MAPKAPK5-AS1 |
| AMOTL1 | DDIT4L | HOXB7 | NAP1L1 | RARRES1 | TMPRSS2 | MARVELD2 |
| AMOTL2 | DDR2 | HOXC10 | NAPA-AS1 | RARRES2 | TMPRSS4 | MBNL1-AS1 |
| ANGPTL6 | DEFB118 | HOXD13 | NAT16 | RASAL2 | TMSB15A | MCPH1-AS1 |
| ANK2 | DENND2A | HOXD8 | NAV3 | RASD1 | TMSB4X | MEIS1-AS2 |
| ANKRD13B | DEPDC4 | HOXD9 | NBL1 | RASEF | TNC | MINOS1-NBL1 |
| ANKRD29 | DES | HPCA | NCBP2L | RASGEF1C | TNFAIP8L3 | MIR4435-2HG |
| ANKRD33 | DFNA5 | HPD | NCCRP1 | RASIP1 | TNFRSF19 | MMP24-AS1 |
| ANKRD35 | DGCR6L | HPDL | NCKAP5 | RASL10B | TNNC2 | NAALADL2 |
| ANKRD65 | DGKI | HPN | NCS1 | RASSF8 | TNNI3 | NDUFA4L2 |
| ANKRD9 | DHRS2 | HPR | NDN | RBFOX2 | TNNT2 | NDUFC2-KCTD14 |
| ANKS1B | DIO3 | HPX | NDRG4 | RBFOX3 | TNS2 | NPHP3-ACAD11 |
| ANO1 | DKK3 | HR | NDUFA5 | RBMS3 | TOB1-AS1 | NPPA-AS1 |
| ANO2 | DLC1 | HRASLS5 | NDUFAF2 | RBMXL2 | TOM1L1 | PARD6G-AS1 |
| ANO7 | DLGAP1 | HRAT92 | NENF | RBP1 | TOMM40 | PITPNA-AS1 |
| ANTXR1 | DLGAP3 | HRCT1 | NES | RBP4 | TP63 | POM121L12 |
| ANXA8 | DLK1 | HRG | NET1 | RBPMS | TPBGL | PPAN-P2RY11 |
| AOX1 | DLL4 | HRH3 | NEU4 | RBPMS2 | TPD52L1 | PRKAR2A-AS1 |
| AP000253.1 | DLX2 | HS3ST3A1 | NEURL1B | RDH8 | TPH2 | PRMT5-AS1 |
| AP3S1 | DLX3 | HS3ST6 | NEURL3 | RDX | TPM4 | PSMG3-AS1 |
| APBA1 | DLX5 | HSD11B1L | NEXN-AS1 | REEP1 | TPO | RAD51L3-RFFL |
| APBB2 | DMD | HSD17B14 | NFASC | REEP2 | TPPP | RAET1E-AS1 |
| APCDD1L | DMKN | HSD17B3 | NFATC4 | REG1A | TPPP2 | RAMP2-AS1 |
| APLN | DNAAF3 | HSD17B6 | NFIB | REG3A | TPRG1 | RASGEF1A |
| APLNR | DNAH10 | HSD3B1 | NGEF | REG3G | TPRX1 | RNF144A-AS1 |
| APOA1 | DNAH11 | HSD3B2 | NIPAL4 | REP15 | TPRXL | RP11-1012A1.4 |
| APOA2 | DNAH2 | HSD3B7 | NKPD1 | RERG | TPSAB1 | RP11-101E13.5 |
| APOA4 | DNAJC22 | HSPA12B | NKX2-1 | RFPL1 | TPSB2 | RP11-103H7.5 |
| APOC1 | DNAJC5B | HSPB6 | NKX2-3 | RFPL2 | TPSD1 | RP11-1072A3.3 |
| APOC2 | DNAJC5G | HSPB7 | NKX2-5 | RFTN2 | TRAV16 | RP11-1079K10.4 |
| APOC3 | DNALI1 | HSPB8 | NKX6-3 | RGMA | TRAV41 | RP11-1094M14.12 |
| APOD | DNASE1L2 | HSPG2 | NLGN1 | RGS11 | TRAV8-3 | RP11-10A14.4 |
| APOE | DNASE1L3 | HTR1A | NLRP10 | RGS21 | TRBV28 | RP11-1100L3.7 |
| APOH | DNLZ | HTR1B | NLRP4 | RGS5 | TRBV7-4 | RP11-1100L3.8 |
| APOL4 | DNMT3B | HTR4 | NMB | RGS7BP | TRHDE | RP11-110G21.1 |
| AQP1 | DNPH1 | HTRA1 | NMNAT2 | RHBDF1 | TRIM15 | RP11-110I1.12 |
| AQP7 | DOC2B | HULC | NNMT | RHBDL3 | TRIM2 | RP11-111M22.3 |
| AR | DOCK1 | HYDIN | NNT-AS1 | RHEBL1 | TRIM29 | RP11-1151B14.4 |
| ARC | DOCK6 | ICA1 | NOS1 | RHOD | TRIM47 | RP11-118A1.2 |
| ARF4-AS1 | DOK5 | ID1 | NOS1AP | RHOJ | TRIM54 | RP11-1212A22.4 |
| ARHGAP36 | DOK6 | ID3 | NOSTRIN | RHOV | TRIM7 | RP11-1223D19.3 |
| ARHGEF15 | DPP6 | IFI27 | NOVA1 | RHPN2 | TRIM73 | RP11-1260E13.4 |
| ARHGEF16 | DPT | IFI27L1 | NOVA2 | RIBC2 | TRIML2 | RP11-128A17.1 |
| ARHGEF25 | DPYS | IFNA4 | NOX4 | RIMBP2 | TRIP10 | RP11-138I1.4 |
| ARMC9 | DPYSL3 | IFNE | NPAS3 | RIMS1 | TRNP1 | RP11-1398P2.1 |
| ARMCX6 | DQX1 | IGF1 | NPFFR2 | RIMS2 | TRO | RP11-140L24.4 |
| ARNT2 | DRC7 | IGF2 | NPHP1 | RIMS3 | TRPA1 | RP11-14J7.6 |
| ARNTL2 | DRD2 | IGFBP2 | NPM2 | RIPK4 | TRPM3 | RP11-14N7.2 |
| ARSJ | DSCAML1 | IGFBP4 | NPNT | RN7SL1 | TSGA10IP | RP11-152P23.2 |
| ARTN | DSCC1 | IGFBP5 | NPR1 | RNASE1 | TSKU | RP11-15H20.6 |
| ASAP3 | DSG1 | IGFBP6 | NPR3 | RNASE10 | TSPAN1 | RP11-160O5.1 |
| ASB10 | DSP | IGKV1D-39 | NPY1R | RNASE7 | TSPAN10 | RP11-161M6.2 |
| ASB4 | DUSP13 | IGKV3-15 | NQO1 | RNASE8 | TSPAN12 | RP11-162A12.2 |
| ASCL4 | DUSP15 | IGLL1 | NR0B2 | RND3 | TSPAN9 | RP11-167N5.5 |
| ASPA | DUSP26 | IGLV10-54 | NR1H3 | RNF150 | TSSK1B | RP11-173P15.3 |
| ASPHD1 | DUSP8 | IGLV2-11 | NR1I3 | RNF186 | TTC7B | RP11-173P15.5 |
| ASS1 | DUX4 | IGLV3-10 | NR2F1 | RNF212B | TTR | RP11-176N18.2 |
| ATP13A5 | DYNC1I1 | IGLV3-25 | NR2F2 | RNF222 | TTYH1 | RP11-178L8.8 |
| ATP1A2 | DZIP1 | IGLV6-57 | NR2F6 | ROBO4 | TUBA1B | RP11-17M16.2 |
| ATP2B2 | DZIP1L | IGSF21 | NR5A1 | ROPN1 | TUBA3C | RP11-182J1.1 |
| ATP2C2 | E2F1 | IL10 | NR5A2 | ROR1 | TUBAL3 | RP11-182J1.3 |
| ATP5EP2 | EBF3 | IL12A | NRARP | RP9 | TUBB3 | RP11-192H23.4 |
| ATP5G2 | EBI3 | IL13RA2 | NRG3 | RPL10 | TUBB6 | RP11-192H23.5 |
| ATP5J | ECI1 | IL17D | NRGN | RPL11 | TUBB8 | RP11-203B9.4 |
| ATP5J2 | ECM1 | IL17RC | NRK | RPL12 | TUBG1 | RP11-203H19.2 |
| ATP6V1B1 | ECSCR | IL17RD | NRL | RPL13 | TWIST1 | RP11-20I23.3 |
| ATRNL1 | ECSCR | IL17REL | NRN1 | RPL13A | TYRO3 | RP11-212D19.4 |
| ATXN8OS | EDDM3A | IL1A | NRSN2 | RPL18 | TYRP1 | RP11-212P7.3 |
| AUNIP | EDDM3B | IL20 | NT5DC4 | RPL18A | U91328.21 | RP11-216B9.6 |
| AVPI1 | EDN3 | IL23A | NT5M | RPL23 | UACA | RP11-216B9.8 |
| AVPR1B | EDNRB | IL25 | NTF3 | RPL27 | UBD | RP11-219B4.5 |
| AXL | EEF1G | IL33 | NTN4 | RPL28 | UBE2C | RP11-21J18.1 |
| AZGP1 | EFCC1 | IL34 | NTNG1 | RPL29 | UBL4B | RP11-21L1.1 |
| B4GALNT1 | EFEMP1 | IL4I1 | NTRK2 | RPL3 | UBQLN3 | RP11-227G15.3 |
| B4GALT2 | EFHB | INAFM2 | NTRK3 | RPL35 | UBQLNL | RP11-229P13.23 |
| BAALC | EFHD1 | INHBA | NTS | RPL36 | UCHL1 | RP11-231C18.3 |
| BAAT | EFNA1 | INHBC | NUAK1 | RPL37A | UCN2 | RP11-244F12.3 |
| BAD | EFNA5 | INMT | NUDT17 | RPL38 | UGT2B7 | RP11-24N18.1 |
| BAMBI | EFNB2 | INPP5J | NUDT8 | RPL4 | UHRF1 | RP11-253E3.3 |
| BANF1 | EFR3B | INTU | NUPR1 | RPL5 | UNC13A | RP11-254F7.3 |
| BARHL2 | EFS | IP6K3 | NUTF2 | RPL7A | UNC13B | RP11-256P1.1 |
| BARX2 | EGFL7 | IQGAP3 | NXF3 | RPPH1 | UNC5B | RP11-259O2.3 |
| BATF3 | EGFLAM | IQSEC3 | NXN | RPRM | UNC5CL | RP1-125I3.2 |
| BBS12 | EGFR | IRF6 | NXPH1 | RPS10 | USH1C | RP11-262H14.4 |
| BBS5 | EGLN3 | IRGC | NYNRIN | RPS11 | USHBP1 | RP11-265D17.2 |
| BCAN | EHD2 | IRX3 | OAZ3 | RPS15 | USP13 | RP11-265N6.1 |
| BCAR1 | EHD3 | IRX5 | OBSL1 | RPS16 | USP2 | RP11-266K4.9 |
| BCAS1 | EIF3F | IRX6 | OIP5-AS1 | RPS17 | USP50 | RP11-273G15.2 |
| BCL2L10 | ELF3 | ISLR | OIT3 | RPS18 | UTF1 | RP11-284H19.1 |
| BCL2L14 | ELN | ISLR2 | OLFML1 | RPS19 | UXT-AS1 | RP11-286N22.6 |
| BCL2L2 | EMCN | ISM2 | OLFML2A | RPS2 | VAMP3 | RP11-290L1.3 |
| BCL6B | EMILIN1 | ITGA10 | OLIG3 | RPS20 | VAMP5 | RP11-294J22.6 |
| BDKRB2 | EML1 | ITGA2B | ONECUT1 | RPS23 | VASH1 | RP11-294N21.2 |
| BEND3 | EMP2 | ITGA9 | OPHN1 | RPS24 | VASH2 | RP11-295P9.3 |
| BEST3 | EMX1 | ITGA9-AS1 | OR10A5 | RPS27A | VCAM1 | RP11-298I3.5 |
| BEX1 | ENAH | ITGBL1 | OR10G2 | RPS28 | VEGFC | RP11-2C24.4 |
| BFSP1 | ENPP6 | ITIH1 | OR10H1 | RPS3 | VGF | RP11-304L19.13 |
| BFSP2 | EPAS1 | ITIH3 | OR10H4 | RPS5 | VGLL3 | RP11-305E6.4 |
| BGN | EPB41L1 | ITIH5 | OR10H5 | RPS6 | VIPR2 | RP11-314N14.1 |
| BHMT | EPHB3 | ITPKA | OR10J3 | RPS6KL1 | VN1R2 | RP11-316M1.12 |
| BHMT2 | EPO | IYD | OR10K2 | RPS7 | VPREB3 | RP11-317N8.5 |
| BISPR | EPPIN | JAG2 | OR10P1 | RRAS | VSNL1 | RP11-321N4.5 |
| BMP2 | ERBB3 | JAM2 | OR10Z1 | RRM2 | VSTM2A | RP11-322E11.2 |
| BMP4 | ERC2 | JPH2 | OR11A1 | RSC1A1 | VSTM2L | RP11-324E6.6 |
| BMP7 | ERG | JPH3 | OR12D3 | RSPH1 | VTN | RP11-325F22.2 |
| BMPER | ERICH3 | JPX | OR14C36 | RTBDN | VWA1 | RP11-326N17.2 |
| BNC1 | ERICH5 | KALRN | OR1A1 | RTKN | VWA5B2 | RP11-343C2.9 |
| BOC | ERRFI1 | KANK3 | OR1D2 | RTN4RL2 | VWA7 | RP11-343J3.2 |
| BOLA3 | ESM1 | KAZALD1 | OR1F1 | RXFP1 | VWF | RP11-346D14.1 |
| BTBD17 | ESRG | KBTBD12 | OR1I1 | RYR2 | WBP5 | RP11-347C12.3 |
| BTNL9 | ESRP2 | KCNA5 | OR1J4 | RYR3 | WBSCR27 | RP11-350G8.9 |
| C11orf16 | ESRRB | KCNB2 | OR1L3 | S100A13 | WDR34 | RP11-35J6.1 |
| C11orf95 | ETNK2 | KCND2 | OR1L8 | S100A14 | WDR87 | RP11-362F19.1 |
| C11orf96 | ETV1 | KCNG4 | OR1N2 | S100A16 | WDR97 | RP11-365O16.3 |
| C12orf57 | ETV2 | KCNIP2 | OR1S2 | S100B | WFDC1 | RP11-366H4.3 |
| C12orf75 | EVA1A | KCNJ11 | OR2A1-AS1 | S1PR2 | WFDC3 | RP11-367G6.3 |
| C14orf105 | EVA1B | KCNJ12 | OR2A25 | SAA2 | WIPF3 | RP11-375N15.2 |
| C14orf37 | EVX1 | KCNJ4 | OR2A5 | SALL4 | WISP2 | RP11-378J18.9 |
| C15orf59 | EXO1 | KCNJ5 | OR2B3 | SAMD14 | WNK4 | RP11-380O24.1 |
| C15orf65 | EXOC3L1 | KCNJ8 | OR2B6 | SAMD4A | WNT11 | RP11-382A20.3 |
| C16orf13 | EXOC3L2 | KCNK15 | OR2D3 | SATB2 | WNT2B | RP11-382B18.1 |
| C16orf45 | EXOC3L4 | KCNK17 | OR2F2 | SAXO1 | WNT3A | RP11-383C5.3 |
| C16orf59 | EYA1 | KCNK4 | OR2G3 | SAXO2 | WNT5A | RP11-397A16.1 |
| C16orf89 | F10 | KCNK9 | OR2L13 | SBK2 | WNT5B | RP11-398H6.1 |
| C17orf74 | F2RL3 | KCNN2 | OR2M2 | SBSN | WSCD1 | RP11-398K22.12 |
| C17orf89 | FABP1 | KCNN3 | OR2M5 | SCARA3 | WT1 | RP11-401P9.5 |
| C17orf96 | FABP3 | KCNQ4 | OR2V2 | SCARA5 | WTIP | RP11-403P17.3 |
| C19orf33 | FABP4 | KCNQ5 | OR2Y1 | SCEL | WWC1 | RP11-404E16.1 |
| C19orf57 | FABP5 | KCNS3 | OR3A1 | SCGB1C1 | WWTR1 | RP11-423H2.3 |
| C19orf73 | FADS6 | KCTD10 | OR4C13 | SCGB1C2 | XIRP2 | RP11-428O18.6 |
| C1orf106 | FAHD2B | KDM4D | OR4C15 | SCGB3A1 | XPNPEP2 | RP11-430B1.2 |
| C1orf115 | FAM107A | KDR | OR4C3 | SCHIP1 | YAP1 | RP11-432B6.3 |
| C1orf145 | FAM109B | KHDRBS3 | OR4C5 | SCIN | YES1 | RP11-433J22.2 |
| C1orf226 | FAM110D | KIAA1024 | OR4D5 | SCML2 | Z83844.1 | RP11-438L7.1 |
| C1orf87 | FAM124A | KIAA1161 | OR4D6 | SCN4A | ZBED3 | RP11-43N16.4 |
| C1QA | FAM127C | KIAA1211 | OR4E2 | SCN8A | ZBED5-AS1 | RP11-449P15.1 |
| C1QB | FAM131B | KIAA1217 | OR4K2 | SCNN1B | ZBED8 | RP11-44K6.3 |
| C1QC | FAM132A | KIAA1456 | OR4N5 | SCOC-AS1 | ZBTB46 | RP11-44N21.4 |
| C1QL4 | FAM132B | KIAA1462 | OR4Q3 | SCUBE1 | ZBTB7C | RP11-455O6.8 |
| C1QTNF1 | FAM13C | KIAA1522 | OR4X2 | SCUBE2 | ZCCHC24 | RP11-45M22.4 |
| C1QTNF5 | FAM149A | KIAA1614 | OR51E1 | SDC1 | ZCWPW2 | RP11-463J10.3 |
| C1QTNF9B | FAM150A | KIAA1644 | OR51F2 | SDCBP2 | ZDHHC1 | RP11-466P24.6 |
| C1S | FAM167A | KIAA1755 | OR51G1 | SEC14L5 | ZDHHC11 | RP11-469H8.8 |
| C20orf144 | FAM167B | KIF17 | OR51G2 | SEC14L6 | ZDHHC11B | RP11-483I13.5 |
| C21orf62 | FAM180B | KIF1A | OR51M1 | SEC16B | ZDHHC9 | RP11-486L19.2 |
| C22orf15 | FAM187A | KIF1C | OR51S1 | SELE | ZFHX4 | RP11-488C13.7 |
| C22orf24 | FAM189A2 | KIF20A | OR52A5 | SEMA3A | ZFP2 | RP11-498C9.13 |
| C22orf46 | FAM207A | KIF26A | OR52B2 | SEMA3B | ZFP37 | RP11-498C9.2 |
| C2CD4C | FAM212A | KIF26B | OR52D1 | SEMA3D | ZFPM2 | RP11-4L24.3 |
| C2orf16 | FAM213A | KIFC3 | OR52E2 | SEMA3F | ZIC4 | RP11-500M8.7 |
| C2orf54 | FAM219A | KIRREL | OR52L1 | SEMA3G | ZKSCAN2 | RP11-501J20.5 |
| C3orf56 | FAM222A | KITLG | OR52N2 | SEMA5A | ZMAT4 | RP11-505K9.4 |
| C3orf70 | FAM24A | KL | OR52W1 | SEMA6C | ZNF112 | RP11-511P7.5 |
| C4BPB | FAM26E | KLF17 | OR56A1 | SEMA6D | ZNF135 | RP11-512M8.5 |
| C4orf48 | FAM27C | KLHDC9 | OR56B1 | SENCR | ZNF165 | RP11-521B24.5 |
| C5orf64 | FAM64A | KLHL33 | OR5A1 | SEPP1 | ZNF205 | RP11-521C20.3 |
| C6orf132 | FAM69B | KLHL35 | OR5AN1 | SEPT7-AS1 | ZNF229 | RP11-529K1.2 |
| C6orf141 | FAM71E1 | KLHL4 | OR5C1 | SEPW1 | ZNF324B | RP11-529K1.3 |
| C6orf223 | FAM81A | KLK1 | OR5D14 | SERPINA3 | ZNF334 | RP11-530C5.1 |
| C7 | FAM81B | KLK10 | OR5I1 | SERPINA5 | ZNF358 | RP11-539L10.3 |
| C8orf34 | FAM83E | KLK11 | OR5M11 | SERPINA6 | ZNF366 | RP11-544I20.2 |
| C8orf4 | FAM83H | KLK15 | OR5P2 | SERPINA9 | ZNF385C | RP11-551L14.4 |
| C8orf82 | FAM84A | KLK2 | OR6A2 | SERPINC1 | ZNF423 | RP11-552M11.4 |
| C9orf16 | FAM86B1 | KLK3 | OR6N1 | SERTAD4 | ZNF462 | RP11-556E13.1 |
| C9orf9 | FAM86C1 | KLK4 | OR6P1 | SFTA3 | ZNF470 | RP11-566K19.6 |
| CA5A | FAM92A1 | KLK6 | OR6T1 | SFTPA2 | ZNF471 | RP11-56L13.3 |
| CABLES1 | FAM95B1 | KLK7 | OR6V1 | SFTPC | ZNF479 | RP11-571E6.3 |
| CABP1 | FAM9C | KNCN | OR7A17 | SGK2 | ZNF502 | RP11-573D15.2 |
| CABYR | FARP1 | KNG1 | OR7A5 | SGSM1 | ZNF536 | RP11-573D15.8 |
| CACNA1C | FAT3 | KRT13 | OR8A1 | SH2D4A | ZNF541 | RP11-598F7.5 |
| CACNA2D1 | FBLIM1 | KRT15 | OR8B4 | SH2D6 | ZNF556 | RP11-619A14.2 |
| CACNG8 | FBLL1 | KRT16 | OR8D1 | SH3BGR | ZNF584 | RP11-629N8.5 |
| CADM2 | FBLN1 | KRT17 | OR9A2 | SH3D19 | ZNF608 | RP11-644F5.10 |
| CADM3 | FBXL22 | KRT18 | OR9A4 | SH3GL3 | ZNF610 | RP11-644F5.11 |
| CADPS | FBXL7 | KRT19 | OR9G1 | SH3PXD2A | ZNF620 | RP11-649E7.5 |
| CADPS2 | FBXO17 | KRT222 | OSR1 | SH3PXD2B | ZNF704 | RP11-660L16.2 |
| CALB1 | FBXO36 | KRT6A | OSR2 | SH3RF2 | ZNF71 | RP11-661A12.7 |
| CALB2 | FBXO43 | KRT7 | OTC | SHANK2 | ZNF77 | RP11-667K14.4 |
| CALCRL | FBXW9 | KRT77 | OVOS2 | SHANK3 | ZNF771 | RP11-67L3.5 |
| CALD1 | FCGBP | KRT78 | OXTR | SHC2 | ZNF774 | RP11-681L8.1 |
| CALHM3 | FCN2 | KRT8 | P4HA2 | SHE | ZNF778 | RP11-691N7.6 |
| CALML3 | FCN3 | KRT80 | PAH | SHROOM2 | ZNF781 | RP11-6N17.6 |
| CAMK1G | FEM1A | KRT86 | PAK3 | SIGLEC11 | ZNF81 | RP11-6O2.4 |
| CAMK2B | FERMT2 | KRTAP10-2 | PAK6 | SIRT4 | ZNF835 | RP11-701B16.2 |
| CAMK2N1 | FFAR3 | KRTAP1-1 | PALD1 | SIX5 | ZNF837 | RP11-701H24.8 |
| CAMKV | FGA | KRTAP3-1 | PALM | SKA1 | ZNF853 | RP11-703G6.1 |
| CAPN11 | FGB | KRTAP4-8 | PALM3 | SKIDA1 | ZNF90 | RP11-706C16.8 |
| CAPN13 | FGD1 | KRTAP5-10 | PALMD | SKOR2 | ZNRF4 | RP11-707G14.1 |
| CARD10 | FGD5 | KRTAP5-2 | PAMR1 | SLAMF9 | ZSCAN10 | RP11-70C1.3 |
| CASKIN2 | FGF1 | KRTAP5-4 | PARD3B | SLC12A5 | ZSCAN20 | RP11-70F11.8 |
| CAV1 | FGF11 | KRTAP5-9 | PARD6G | SLC13A3 | ZSCAN31 | RP11-731D1.4 |
| CAV2 | FGF12 | KRTAP6-3 | PARVA | SLC16A11 | AC003002.4 | RP11-731F5.1 |
| CBR3 | FGF17 | KRTAP8-1 | PATE3 | SLC17A3 | AC003075.4 | RP11-732A19.8 |
| CBR3-AS1 | FGF18 | KSR2 | PAX2 | SLC22A11 | AC004448.5 | RP11-73M18.2 |
| CBX2 | FGF2 | L1TD1 | PAX4 | SLC22A12 | AC004540.4 | RP11-744N12.3 |
| CCDC103 | FGF20 | L3MBTL4 | PAX6 | SLC22A7 | AC004813.1 | RP11-746M1.1 |
| CCDC108 | FGFR4 | LAD1 | PCAT19 | SLC22A8 | AC004837.4 | RP11-74E22.8 |
| CCDC114 | FGG | LAGE3 | PCDH15 | SLC23A1 | AC004853.1 | RP11-758P17.3 |
| CCDC116 | FHDC1 | LAMA3 | PCDH17 | SLC25A15 | AC005037.3 | RP11-77H9.5 |
| CCDC136 | FHL1 | LAMA4 | PCDH18 | SLC25A27 | AC005076.5 | RP1-178F15.4 |
| CCDC155 | FIGN | LAMA5 | PCDH7 | SLC25A47 | AC005253.4 | RP1-178F15.5 |
| CCDC184 | FILIP1 | LAMB2 | PCDHA12 | SLC25A53 | AC005262.2 | RP11-78F17.1 |
| CCDC188 | FILIP1L | LAMB3 | PCDHAC2 | SLC26A10 | AC005339.2 | RP11-792A8.4 |
| CCDC3 | FKBP10 | LAMC3 | PCDHB16 | SLC29A4 | AC005614.5 | RP11-793H13.3 |
| CCDC40 | FKBP7 | LAMP5 | PCDHB3 | SLC2A10 | AC005625.1 | RP11-805F19.3 |
| CCDC58 | FLJ22447 | LAPTM4B | PCDHB4 | SLC2A4 | AC005779.2 | RP11-805L22.3 |
| CCDC74B | FLJ40194 | LARP6 | PCDHGA1 | SLC2A5 | AC005786.7 | RP11-815J21.2 |
| CCDC78 | FLNC | LAYN | PCDHGC3 | SLC34A1 | AC006449.2 | RP11-81H14.2 |
| CCDC80 | FLRT2 | LCN12 | PCGF2 | SLC35D3 | AC006538.4 | RP11-822E23.8 |
| CCDC81 | FLT1 | LCTL | PCK1 | SLC35E4 | AC006547.13 | RP11-848P1.4 |
| CCDC85B | FMN2 | LDB2 | PCP2 | SLC35G3 | AC006946.15 | RP11-856F16.2 |
| CCDC85C | FMO2 | LEFTY1 | PDAP1 | SLC35G5 | AC007246.3 | RP11-856M7.2 |
| CCL11 | FMO3 | LGALS4 | PDCD1LG2 | SLC35G6 | AC007249.3 | RP11-864I4.1 |
| CCL13 | FN1 | LGI4 | PDE10A | SLC44A4 | AC007283.5 | RP11-867G23.4 |
| CCL14 | FN3K | LHFP | PDE11A | SLC4A11 | AC007679.3 | RP11-874J12.4 |
| CCL19 | FNDC4 | LHX5 | PDE1A | SLC52A3 | AC007906.1 | RP11-876N24.4 |
| CCL2 | FNDC5 | LHX6 | PDE1C | SLC5A1 | AC008074.1 | RP11-879F14.2 |
| CCL21 | FOLH1 | LHX9 | PDE2A | SLC5A5 | AC009005.2 | RP11-885N19.6 |
| CCL22 | FOXA3 | LIF | PDE3A | SLC6A1 | AC009133.21 | RP11-88H9.2 |
| CCL3L3 | FOXC1 | LIFR | PDE4C | SLC6A13 | AC009758.1 | RP11-90J7.3 |
| CCM2L | FOXC2 | LILRB5 | PDE7B | SLC6A16 | AC010084.1 | RP11-90P5.2 |
| CCNA2 | FOXD2 | LIMCH1 | PDGFB | SLC7A10 | AC010226.4 | RP11-91A18.4 |
| CCNB2 | FOXF1 | LIMS2 | PDGFRA | SLC7A2 | AC011558.5 | RP11-923I11.3 |
| CCND1 | FOXI2 | LINC00094 | PDHA2 | SLC7A4 | AC012146.7 | RP11-930O11.1 |
| CCNI2 | FOXL1 | LINC00176 | PDIA2 | SLC8A2 | AC012507.4 | RP11-930P14.2 |
| CCNO | FOXL2 | LINC00239 | PDLIM1 | SLC8A3 | AC015688.3 | RP11-93B14.6 |
| CCR10 | FOXN4 | LINC00264 | PDLIM3 | SLC9A3R2 | AC016700.5 | RP11-95F22.1 |
| CD164L2 | FOXP2 | LINC00483 | PDPN | SLC9A4 | AC016757.3 | RP11-96K19.5 |
| CD209 | FREM1 | LINC00534 | PDZD7 | SLCO2A1 | AC018816.3 | RP11-96O20.4 |
| CD24 | FRMD1 | LINC00639 | PDZRN3 | SLCO2B1 | AC022182.3 | RP11-977G19.11 |
| CD276 | FRMD6 | LINC00665 | PEG3 | SLIT3 | AC024592.12 | RP11-97O12.7 |
| CD300LG | FRMPD1 | LINC00710 | PERM1 | SMAD6 | AC026248.1 | RP11-982M15.2 |
| CD34 | FSCN1 | LINC00861 | PERP | SMAD9 | AC037459.4 | RP11-982M15.6 |
| CD3EAP | FSD1 | LINC00869 | PF4V1 | SMARCA1 | AC046143.3 | RP11-99J16_A.2 |
| CD5L | FST | LINC00905 | PFN4 | SMIM10 | AC068533.7 | RP1-253P7.4 |
| CDC20 | FTCD | LINC00923 | PGF | SMIM10L1 | AC069277.2 | RP13-131K19.6 |
| CDC25A | FTCDNL1 | LINC00958 | PGK2 | SMKR1 | AC069368.3 | RP1-317E23.6 |
| CDC42EP5 | FTH1 | LINC00967 | PGLYRP2 | SMO | AC073254.1 | RP13-514E23.2 |
| CDCA5 | FTL | LINC01088 | PHACTR3 | SMOC1 | AC074117.10 | RP13-580F15.2 |
| CDCP1 | FUT1 | LINC01116 | PHKA1 | SMTN | AC078883.3 | RP13-582O9.6 |
| CDH11 | FUT6 | LINC01151 | PHLDA3 | SMTNL2 | AC079742.4 | RP13-753N3.1 |
| CDH13 | FXYD1 | LINC01160 | PHLDB1 | SNAI2 | AC079767.4 | RP13-753N3.3 |
| CDH20 | FXYD2 | LINC01420 | PHYHIP | SNCAIP | AC090498.1 | RP1-41C23.2 |
| CDH5 | FXYD3 | LINC01558 | PIEZO2 | SNCB | AC093323.3 | RP1-59D14.1 |
| CDH7 | FZD10 | LINC01565 | PIF1 | SNCG | AC093627.8 | RP1-59D14.9 |
| CDHR4 | FZD4 | LINC01588 | PIGY | SNHG11 | AC093818.1 | RP1-67K17.3 |
| CDK1 | FZD8 | LINGO1 | PIGZ | SNHG14 | AC103564.7 | RP1-78O14.1 |
| CDKL3 | G6PC | LINGO4 | PINLYP | SNHG17 | AC108004.3 | RP1-86C11.7 |
| CDKL4 | G6PC2 | UPC | PIPOX | SNHG19 | AC108488.4 | RP1-90J20.8 |
| CDKN2A | GABRE | LIPG | PITPNM3 | SNHG23 | AC109486.1 | RP3-422G23.4 |
| CDO1 | GABRP | LIPH | PITX1 | SNTG1 | AC112721.1 | RP3-467D16.3 |
| CDR1-AS | GADL1 | LKAAEAR1 | PKD2L2 | SNTG2 | AC114273.3 | RP3-508I15.18 |
| CDR2L | GAL3ST1 | LLPH-AS1 | PKDCC | SNX7 | AC114730.3 | RP3-508I15.21 |
| CDRT15 | GALNT15 | LMAN1L | PKIG | SOBP | AC114752.3 | RP4-549L20.3 |
| CDT1 | GALNT16 | LMCD1 | PKLR | SOD3 | AC132217.4 | RP4-559A3.7 |
| CEACAM5 | GALNT18 | LMNA | PKNOX2 | SORBS2 | ADAMTS18 | RP4-583P15.15 |
| CECR2 | GALNT9 | LMO3 | PKP3 | SORCS1 | ADAMTS9-AS1 | RP4-622L5.2 |
| CELF4 | GALNTL6 | LNX1 | PKP4 | SOST | AE000661.37 | RP4-673M15.1 |
| CEMIP | GAP43 | LOR | PLA1A | SOX17 | AF131216.5 | RP4-738P15.6 |
| CENPB | GAPDH | LOX | PLA2G2A | SOX18 | AL513523.2 | RP4-756G23.5 |
| CENPM | GAREM | LOXL2 | PLAT | SOX5 | ANKRD20A1 | RP4-761J14.8 |
| CEP112 | GAREML | LPL | PLAU | SOX7 | ANKRD33B | RP5-1050D4.3 |
| CEP170B | GATA4 | LPO | PLCB4 | SOX8 | ANKRD34A | RP5-1113E3.3 |
| CES3 | GATA5 | LRIG3 | PLCD3 | SP7 | AP000476.1 | RP5-1139B12.3 |
| CETP | GATA6 | LRP11 | PLCD4 | SPA17 | AP000487.5 | RP5-1142A6.8 |
| CFAP126 | GC | LRRC20 | PLCH1 | SPACA4 | AP000654.4 | RP5-864K19.6 |
| CFAP157 | GCGR | LRRC24 | PLCXD3 | SPAG4 | AP001189.4 | RP5-874C20.8 |
| CFAP69 | GCK | LRRC26 | PLEKHA4 | SPARCL1 | AP003068.23 | RP5-877J2.1 |
| CFAP77 | GCKR | LRRC30 | PLEKHA6 | SPATA24 | AP003068.9 | RP5-887A10.1 |
| CFHR1 | GCOM1 | LRRC32 | PLEKHG4B | SPATA8 | AP003419.16 | RP5-940J5.9 |
| CFI | GCSH | LRRC36 | PLEKHG5 | SPC24 | AP003774.4 | RP5-956O18.3 |
| CGN | GEM | LRRC3C | PLEKHH1 | SPDYC | APOC4-APOC2 | RPARP-AS1 |
| CGNL1 | GFAP | LRRC46 | PLEKHH3 | SPEM1 | ARAP1-AS1 | SDCBP2-AS1 |
| CHAC1 | GFPT2 | LRRC49 | PLK2 | SPHK1 | ARHGAP22 | SERPINA12 |
| CHADL | GFY | LRRC52 | PLLP | SPIC | ARHGAP23 | SERTAD4-AS1 |
| CHDH | GGT5 | LRRC74A | PLPP1 | SPINK5 | ARHGAP28 | ST6GALNAC1 |
| CHL1 | GHR | LRRC75B | PLPP2 | SPOCK1 | ARHGAP29 | STON1-GTF2A1L |
| CHMP4C | GHSR | LRRTM1 | PLPP3 | SPP1 | ARHGEF28 | SYNJ2BP-COX16 |
| CHP2 | GINS4 | LRRTM4 | PLPPR3 | SPR | ARHGEF39 | SYS1-DBNDD2 |
| CHRD | GIPC3 | LSAMP | PLS3 | SPRED3 | ATP13A5-AS1 | TCEB3-AS1 |
| CHRDL1 | GJA1 | LSINCT5 | PNCK | SPRR1B | ATP6V0E2-AS1 | TGIF2-C20orf24 |
| CHRDL2 | GJA4 | LSM2 | PNLIPRP1 | SPRR2A | BBOX1-AS1 | TIPARP-AS1 |
| CHRM3 | GJA5 | LURAP1 | PNMA2 | SPRR2D | BHLHE40-AS1 | TM4SF19-AS1 |
| CHRNA2 | GJB1 | LY6G6C | PNMT | SPRR2E | BOLA3-AS1 | TMEM150C |
| CHRNA4 | GJB4 | LY6G6F | PODN | SPRR2F | BRWD1-AS2 | TMEM178A |
| CHST1 | GJB7 | LY6H | PODNL1 | SPRR3 | C1QTNF9B-AS1 | TMEM189-UBE2V1 |
| CHST6 | GJC1 | LY6K | PODXL | SPRY1 | C6orf47-AS1 | TMEM255B |
| CHST8 | GJD3 | LYNX1 | POLN | SPRY4 | CCL15-CCL14 | TMEM256-PLSCR3 |
| CIART | GLDN | LYPD3 | PON1 | SPRYD4 | CDC42BPG | TMPO-AS1 |
| CILP2 | GLI2 | LYPD5 | POT1-AS1 | SPTBN4 | CEACAM18 | TRHDE-AS1 |
| CISD3 | GLIS2 | LYPD6 | POU6F2 | SRCIN1 | CH17-270A2.2 | TRIM52-AS1 |
| CKM | GLIS3 | LZTS2 | PP14571 | SRL | CMB9-22P13.1 | WDR11-AS1 |
| CKS1B | GLP2R | M1AP | PPARG | SRY | CNTN4-AS1 | ZFP91-CNTF |
| CKS2 | GLYATL1 | MAATS1 | PPFIA2 | SSC5D | CRTC3-AS1 | ZMYM4-AS1 |
| CLDN11 | GLYATL2 | MAGEA4 | PPFIBP1 | SSTR5 | CTA-363E6.7 | ZNF337-AS1 |
| CLDN14 | GMDS-AS1 | MAGEA8 | PPIC | SSUH2 | CTB-186H2.3 | ZNF436-AS1 |
| CLDN19 | GNA11 | MAGI1 | PPM1J | ST8SIA5 | CTB-25B13.12 | ZNF503-AS2 |
| CLDN25 | GNA14 | MAGI2 | PPP1R14A | STAB2 | CTB-31O20.3 | ZNF529-AS1 |
| CLDN3 | GNAI1 | MAGIX | PPP1R14B | STAC | CTB-31O20.6 | ZNF561-AS1 |
| CLDN4 | GNB2L1 | MAL2 | PPP1R14C | STAC2 | CTB-43P18.1 | ZNF571-AS1 |
| CLDN5 | GNG12 | MALL | PPP1R1A | STAP2 | CTC-203F4.2 | ZNF667-AS1 |
| CLEC14A | GNG8 | MAMDC2 | PPP1R26 | STC1 | CTC-250114.3 | ZNF790-AS1 |
| CLEC2L | GOLGA6B | MAMSTR | PPP1R3C | STEAP3 | CTC-260E6.6 | ZSCAN16-AS1 |
| CLEC3B | GOLGA6L2 | MAP1B | PPP2R2C | STKLD1 | CTC-360G5.6 | ARMCX5-GPRASP2 |
| CLEC4G | GP1BB | MAP1LC3C | PPP5D1 | STOX2 | CTC-360G5.9 | BCL2L2-PABPN1 |
| CLEC4M | GP9 | MAP2 | PPY | STRA6 | CTC-429P9.1 | BLOC1S5-TXNDC5 |
| CLIC4 | GPC5 | MAPK10 | PRADC1 | STRC | CTC-455F18.1 | LL22NC03-63E9.3 |
| CMTM5 | GPIHBP1 | MAPK11 | PRAMEF17 | STXBP6 | CTC-471J1.8 | LL22NC03-75H12.2 |
| CNFN | GPNMB | MAPK12 | PRAMEF8 | STYK1 | CTC-543D15.8 | PTGES3L-AARSD1 |
| CNIH3 | GPR1 | MAPK15 | PRAP1 | SUGCT | CTC-559E9.1 | RPL36A-HNRNPH2 |
| CNKSR3 | GPR12 | MASP1 | PRC1-AS1 | SULF1 | CTC-564N23.2 | XXbac-B135H6.15 |
| CNN3 | GPR142 | MAT1A | PRDM16 | SULT1C4 | CTD-2012K14.4 | XXbac-B476C20.9 |
| CNRIP1 | GPR152 | MATN1 | PRDM7 | SUSD2 | CTD-2024P10.1 | XXbac-BPG32J3.19 |
| CNTN1 | GPR161 | MATN4 | PREX2 | SWI5 | CTD-2026K11.4 | XXbac-BPG32J3.20 |
| CNTN4 | GPR17 | MB | PRICKLE2 | SYBU | CTD-2031P19.5 | CTD-2207O23.11 |
| CNTN5 | GPR176 | MC2R | PRIMA1 | SYCE1 | CTD-2033A16.3 | CTD-2196E14.7 |
| COBL | GPR179 | MCAM | PRKCDBP | SYCE1L | CTD-2135J3.4 | CTD-2514K5.4 |
| COL12A1 | GPR32 | MCAT | PRNT | SYDE1 | CTD-2147F2.1 | CTC-338M12.4 |
| COL14A1 | GPR4 | MCF2L2 | PROB1 | SYN2 | CTD-2184D3.6 | |
| COL15A1 | GPR45 | MDFI | PROC | SYN3 | SEPT4 | SEPT10 |
| COL1A1 | GPR62 | MDGA2 | PRODH2 | SYNDIG1 | MARCH2 | MARCH11 |
| COL1A2 | GPR78 | ME3 | PROK1 | SYNPO | XXbac-BPG181M17.5 | XXbac-BPG249D20.9 |
| COL27A1 | GPR85 | MEA1 | PROKR2 | SYNPO2 | XXbac-BPG181M17.6 | XXbac-BPG252P9.10 |

TABLE 10
Low-stringency non-blood genes detected in cell-free mRNA

| | | | | | | |
|---|---|---|---|---|---|---|
| NNMT | KCNS3 | C19orf73 | FAM71E2 | RBFOX1 | RP11-73M18.8 | RP11-138I1.4 |
| NFIB | ROR1 | FAM171A2 | HECW1 | COL23A1 | CTB-147C22.8 | RP11-298I3.5 |
| RPL7A | JPX | LINC00176 | OR1E2 | ARHGEF5 | RP5-1092A3.4 | RP11-433J22.2 |
| LDB2 | PIF1 | ARAP1-AS1 | GNGT1 | ROS1 | RP11-342M21.2 | AP003068.23 |
| CDR1-AS | NR1I3 | C15orf59 | GSX2 | CP | RP11-171I2.2 | XXbac-BPG249D20.9 |
| ADIRF | APLN | C1orf145 | GAS1 | WNT16 | RP11-64K12.8 | RP11-70C1.3 |
| RAMP3 | TPPP | IGLV10-54 | ADM2 | MTMR7 | RP11-420L9.5 | CTD-2231E14.8 |
| CALD1 | ANKRD65 | KRTAP10-2 | YBX2 | HOXA11 | RP13-36G14.4 | PTGES3L-AARSD1 |
| RASSF8 | KRT15 | C1orf106 | MGC45922 | THSD7A | RP11-259K5.2 | RP11-501J20.5 |
| SOX18 | KLHL4 | ZDHHC11B | CAPS | KRT33A | RP11-568J23.6 | CTD-3092A11.2 |
| NOSTRIN | GABRE | IGLV6-57 | GTF2IRD1 | CACNG3 | RP11-1008C21.1 | RP11-815J21.2 |
| LMCD1 | NOVA1 | C6orf141 | OR1E1 | CACNA1G | RP11-498E2.9 | SYNJ2BP-COX16 |
| APLNR | EFCC1 | KRTAP5-2 | GOLGA8J | LGALS14 | ANKRD34C-AS1 | BCL2L2-PABPN1 |
| LIMS2 | PAX6 | H1FX-AS1 | OR10A7 | PROM1 | RP11-313P18.1 | RP11-382A20.3 |
| TM4SF1 | KCNJ8 | ADAMTS18 | UPP2 | SLC13A2 | RP11-750H9.7 | RP11-529K1.3 |
| DOCK1 | SPRR1B | ZMYM4-AS1 | NLRP11 | SYN1 | CTD-3065B20.3 | CTD-2135J3.4 |
| PLEKHA4 | MUC7 | TMEM178B | MRGPRX4 | TFAP2D | RP11-932O9.8 | MIR4435-2HG |
| NUPR1 | GPR161 | WASF3-AS1 | MCHR1 | PLEKHG6 | CTD-2132N18.4 | RP11-1012A1.4 |
| RASIP1 | FGFR4 | TMEM132E | NOS2 | PAX7 | RP11-95D17.1 | RP11-982M15.6 |
| MAGI1 | CCDC85C | TRBV11-2 | FAM216B | FMO1 | RP11-382A20.5 | RP5-864K19.6 |
| CCL14 | LYNX1 | CDRT15L2 | DNAH9 | SLC6A7 | CTB-12A17.3 | RP4-673M15.1 |
| ADH1B | KCNQ5 | C10orf126 | TEAD3 | NR1H4 | RP11-812E19.9 | RP11-1100L3.7 |
| RPS19 | ZNF781 | HAND2-AS1 | CPA4 | DPEP1 | RP11-120K9.2 | RP11-346D14.1 |
| FABP4 | DNAH11 | ARHGEF33 | TAPT1-AS1 | ISL1 | RP11-499F3.2 | RP4-583P15.15 |
| GP9 | CPNE4 | LINC00518 | IL20RA | CLCA1 | RP11-485O10.3 | RP3-508I15.18 |
| DLC1 | TDRD10 | UBL7-AS1 | C10orf53 | SLC7A9 | RP11-236L14.1 | CTD-2336O2.1 |
| CAV1 | GRB7 | C11orf72 | THAP10 | C8B | RP11-1069G10.2 | RP11-173P15.5 |
| ANO2 | RAB3A | KRTAP5-6 | OR2T12 | GABRA1 | CTC-429P9.3 | RP11-681L8.1 |
| TTR | ACTG2 | LINC00632 | MBOAT4 | LRRC7 | RP11-324D17.2 | RP11-192H23.4 |
| KANK3 | MUC3A | TMEM200C | CASC2 | MYOC | CTD-2521M24.10 | RP11-571E6.3 |
| NR5A1 | ETV2 | KRTAP5-1 | OR2G2 | HOXC8 | RP11-507J18.2 | RP11-449P15.1 |
| ALDOB | THSD4 | KRTAP10-1 | NIM1K | C6 | CTC-518B2.9 | CTD-2540B15.13 |
| AQP1 | ERC2 | MIR7515HG | UMODL1 | GUCA2B | RP11-256I23.1 | CTD-2207O23.11 |
| BGN | NEURL3 | KLHL6-AS1 | GUCA1A | EPHA3 | CTD-3105H18.16 | TMEM189-UBE2V1 |
| S100A13 | ABCA8 | LINC01559 | KIAA2022 | BRINP1 | RP11-400N9.1 | RP11-44K6.3 |
| PTRF | HPN | C17orf47 | LINC00469 | DCT | RP11-69G7.1 | CTC-471J1.8 |
| LZTS2 | HOXA7 | MGC16275 | CDH15 | NALCN | RP11-151N17.1 | CTC-260E6.6 |
| CXCL12 | INHBA | LINC00963 | TRIM72 | CST9L | RP11-685G9.2 | IQCJ-SCHIP1 |
| LIMCH1 | HCG15 | SPATA31D1 | PRR29-AS1 | PLA2G3 | RP11-66B24.4 | CTD-2196E14.7 |
| PDLIM1 | PRODH2 | C15orf32 | CRYGN | SOX10 | CTD-2651B20.3 | CTD-2267D19.3 |
| AFAP1L2 | DUX4 | IGHV3-16 | SRRM3 | NEFH | RP11-540O11.4 | RP11-1212A22.4 |
| CLEC3B | WTIP | IGHV3-20 | CLVS1 | CABP7 | RP11-259A24.1 | RP11-6N17.6 |
| TM4SF18 | GPR32 | KRTAP4-7 | LINC00982 | PNPLA3 | CTD-2583A14.9 | RAD51L3-RFFL |
| APBB2 | FOXA3 | IGHV1-45 | METTL24 | CACNA1I | RP11-351M8.2 | RP1-178F15.5 |
| GJA5 | PDPN | KRTAP4-2 | FIBIN | SIX4 | RP4-639J15.3 | CTA-363E6.7 |
| PDAP1 | HOXA3 | SLC25A18 | OR51H1 | SERPINA4 | RP11-37C7.1 | CTC-338M12.4 |
| CLIC4 | PSG8 | KRTAP4-11 | RBP3 | BDKRB1 | RP11-521C20.2 | AC074117.10 |
| ALB | CAMK2N1 | KRTAP2-3 | NRIP2 | HNF4A | RP11-253M7.6 | RP11-265D17.2 |
| RPS28 | C1orf87 | KRTAP5-7 | TCERG1L | RIMS4 | CTD-3131K8.2 | RP1-59D14.9 |
| LPL | EFHB | KRTAP29-1 | C15orf56 | NKAIN4 | CTD-2012M11.3 | RP11-343C2.9 |
| NET1 | PCK1 | C12orf60 | MAGEB6 | ANGPT4 | PLA2G4C-AS1 | MAPKAPK5-AS1 |
| PTCRA | XPNPEP2 | PRAMEF14 | C2orf57 | OXT | CTD-2647L4.5 | RP11-732A19.8 |
| FGD5 | ZNF502 | ZSCAN5CP | RTN4R | CST4 | RP11-120K24.5 | BLOC1S5-TXNDC5 |
| CLDN5 | PON1 | PTPRG-AS1 | DEFB125 | SCTR | RP11-244F12.2 | RP11-403P17.3 |
| CCM2L | VCAM1 | LINC00211 | CSRP3 | H2BFM | RP11-46C24.6 | ARMCX5-GPRASP2 |
| PCGF2 | SP7 | SYNDIG1L | OR6B3 | PAGE4 | RP11-244G12.1 | RP11-746M1.1 |
| KDR | GCSH | IGLV3-12 | CTXN1 | SRPX | CTC-518P12.6 | RP11-864I4.1 |
| SYNPO | EBI3 | KRTAP12-3 | CLCA4 | GLRA2 | CTD-3157E16.1 | RP11-45M22.4 |
| APOH | ELF3 | IGLV5-37 | SYT13 | MCF2 | RP11-1090M7.1 | RP11-162A12.2 |
| PITPNM3 | OR10J3 | VAC14-AS1 | UCN3 | CACNA1F | RP5-1119A7.17 | RP11-15H20.6 |
| ASS1 | CCDC58 | RORA-AS1 | CCDC185 | SYP | RP5-1110E20.1 | RP11-367G6.3 |
| ABLIM3 | LGALS4 | KRTAP19-3 | MYOD1 | KCND1 | RP11-473C18.3 | RP11-73M18.2 |
| CGNL1 | CREB3L4 | PSORS1C1 | SHISA3 | OPN1LW | RP11-723G8.2 | RP11-284H19.1 |
| H19 | KLK7 | LINC00887 | AQP11 | RS1 | LA16C-366D1.3 | XXbac-BPG252P9.10 |
| HBD | OR2L13 | LINC00847 | SLITRK1 | GPR50 | CTD-2350C19.2 | RP11-91A18.4 |
| ERG | SEMA6C | HOXA-AS3 | SH2D4B | VGLL1 | RP11-775G23.1 | RP1-125I3.2 |
| EGFL7 | PLEKHG5 | LINC00856 | IBSP | ITIH6 | RP11-953B20.1 | RP11-266K4.9 |
| PALMD | OAZ3 | LINC00336 | DNAJC28 | ZDHHC15 | RP11-781P6.1 | RP11-227G15.3 |
| BCL2L2 | HOXAIO | SMAD1-AS1 | AMER3 | CRYBB3 | RP11-344B2.2 | CH17-270A2.2 |
| DPYSL3 | SCGB1C1 | SLC22A31 | ZNF114 | IGF2-AS | RP11-143K11.5 | RP4-761J14.8 |
| APOE | GALNT16 | KRTAP10-8 | MUSK | AMELY | RP11-161M6.5 | CTD-3214H19.4 |
| RNASE1 | DGKI | LINC00824 | SOX15 | SYDE2 | CH17-360D5.1 | RP11-573D15.2 |
| PALM | OSR1 | CDC42-IT1 | KLK14 | CFHR2 | RP11-769O8.3 | CTD-2545M3.2 |
| AIF1L | TMSB15A | LINC01090 | SLC18A1 | CACNA1S | RP11-474D1.4 | CTD-3214H19.6 |
| SMTN | TPSAB1 | ABCA9-AS1 | CASR | LRP2 | RP11-13N13.6 | RP11-466P24.6 |
| RAMP2 | CRNN | RTCA-AS1 | ZNRF3-AS1 | EPYC | RP11-5A19.5 | RP11-316M1.12 |
| NES | PAH | LINC01198 | SPATA31E1 | NKAIN1 | RP4-614O4.11 | ZSCAN16-AS1 |
| HIF3A | SLC6A13 | SLC22A24 | ODF3 | COL16A1 | RP11-640I15.1 | CTD-2207O23.12 |
| NRN1 | SLC52A3 | NEBL-AS1 | KLK8 | AQP6 | RP11-757F18.5 | RP11-552M11.4 |
| BCL6B | ZBTB7C | DNAH100S | SOX30 | PPEF1 | RP11-41O4.1 | CTB-31O20.3 |
| SPARCL1 | TRBV28 | LINC01363 | FAM170B | TMPRSS11E | RP11-46D6.1 | RP4-549L20.3 |
| TJP1 | M1AP | LINC00278 | SFT2D3 | TFAP2C | RP11-432I5.8 | CTD-2396E7.10 |
| EFNB2 | JAG2 | KLF3-AS1 | SLN | SULT2B1 | RP11-403P17.4 | RP13-753N3.3 |
| FAM212A | SGSM1 | C16orf47 | CYP2D6 | SLC15A1 | RP11-647F2.2 | CTC-559E9.1 |
| COX7A1 | GPR142 | LINC00853 | RASL10A | F11 | RP11-304L19.8 | RP11-273G15.2 |
| NR2F2 | SLC2A10 | C10orf99 | TAS1R3 | CFAP61 | CTD-2555A7.3 | RP11-430B1.2 |
| HSPA12B | FOXP2 | C1orf210 | IGKV1D-13 | KCNH4 | RP11-457M11.5 | RP11-343J3.2 |
| CD34 | PRDM7 | KIAA0408 | RASD2 | OTUB2 | RP11-883A18.3 | CTD-2651B20.1 |
| NDRG4 | KLK10 | LINC01013 | PYDC1 | SLC26A4 | AC010761.10 | RP11-110G21.1 |
| C9orf16 | CHRDL1 | CRYM-AS1 | WNT10B | SMPX | EEF1E1-BLOC1S5 | CTD-2033A16.3 |
| MECOM | EFNA1 | CERS6-AS1 | PNPLA5 | CMA1 | RP11-357N13.3 | XXbac-BPG181M17.6 |
| ENAH | INPP5J | KRTAP5-3 | TAS2R1 | DAZL | RP11-352D13.6 | RP11-744N12.3 |
| LMNA | CPNE6 | LINC00352 | TMEM266 | NXPE1 | RP11-309H21.3 | SERTAD4-AS1 |
| ID1 | FAM131B | C9orf163 | DYDC2 | CYP26A1 | RP11-69H7.3 | RP11-21J18.1 |
| PLS3 | BEST3 | SMIM2-AS1 | MUC15 | MYO3A | RP11-138C9.1 | ST0N1-GTF2A1L |
| FTL | UNC5B | SZT2-AS1 | GSC | TREM2 | RP11-76K13.3 | RP11-231C18.3 |
| MSRB3 | S100A14 | OGFR-AS1 | VSX1 | PGC | RP11-26L20.4 | GS1-124K5.4 |
| WBP5 | IRX6 | LINC01315 | OR4K15 | NCR2 | RP11-199F11.2 | RP1-86C11.7 |
| FHDC1 | LOX | KRTAP24-1 | OR4K14 | MLN | RP11-595B24.2 | CTD-2270L9.4 |
| TEAD4 | NCS1 | UPK1A-AS1 | VRTN | A4GNT | RP11-338L22.2 | RP11-256P1.1 |
| FCN3 | OR2G3 | C12orf77 | UMOD | CRYGD | CH17-351M24.1 | RP11-982M15.2 |
| TPM4 | OLIG3 | SLC30A10 | EEF1A2 | ATP10B | CTD-2135D7.3 | CTD-2531D15.5 |
| FAM107A | PRAP1 | LINC01419 | OR5AU1 | POU2F3 | RP11-235E17.2 | RP11-216B9.8 |
| LAMA4 | TPRXL | CATSPER4 | ADAMDEC1 | IL36A | LA16C-390H2.4 | RP3-467D16.3 |
| IGF2 | ANO7 | LINC01099 | SPEF1 | IL36B | CTD-3195I5.4 | RP11-874J12.4 |
| UACA | OR10A5 | HOXA-AS2 | MIOX | CFC1 | RP11-85G18.6 | RP11-35J6.1 |
| RPS11 | CDKL4 | TMPRSS11B | ANKK1 | OR1J1 | RP5-1050D4.5 | CTD-2012K14.4 |
| CNN3 | RNASE10 | C1orf229 | PLGLB2 | OR13C9 | RP11-667K14.3 | RP4-738P15.6 |
| RPS18 | IRX5 | SSBP3-AS1 | ADRA1B | WDR38 | RP11-565F19.4 | RP11-511P7.5 |
| ID3 | HOXC10 | C6orf201 | TTLL6 | LMX1B | RP11-1113L8.1 | RP11-498C9.2 |
| PTMA | ANXA8 | JAZF1-AS1 | ANGPTL3 | IGFBPL1 | RP11-64J4.2 | RP11-118A1.2 |
| EHD2 | FAHD2B | MEF2C-AS1 | SYT4 | CLPS | CTD-2561B21.3 | RP11-326N17.2 |
| IGFBP5 | OR51S1 | LINC00970 | IRX1 | C6orf52 | RP13-1032I1.10 | RP5-887A10.1 |
| TMSB4X | LY6H | LY86-AS1 | TMC7 | FGFBP1 | RP11-408H20.3 | RP11-667K14.4 |
| SEPW1 | THNSL2 | LINC00955 | NPBWR2 | MMP20 | RP11-849F2.7 | RP11-77H9.5 |
| SHANK3 | MUC12 | OLMALINC | LONRF2 | MMP27 | RP11-350O14.18 | C6orf47-AS1 |
| KIFC3 | C9orf9 | SNCA-AS1 | BARHL1 | C11orf70 | CTC-479C5.12 | RP11-404E16.1 |
| WWTR1 | CCDC108 | C14orf180 | KRT74 | SLC28A2 | RP11-680F20.12 | RP11-551L14.4 |
| GIPC3 | PERM1 | LINC00961 | CBARP | ENPEP | RP11-68I3.4 | RP5-1139B12.3 |
| RAB13 | OR6T1 | MIR100HG | KRT4 | SPTBN5 | RP11-44F14.2 | MINOS1-NBL1 |
| EPAS1 | TPPP2 | LINC01278 | PCDH11Y | SULT6B1 | RP11-435I10.4 | XXbac-B476C20.9 |
| KLK1 | ACCSL | Z84812.4 | SDR9C7 | ABCG5 | RP1-253P7.1 | CTD-2369P2.12 |
| PROX1 | ADH1A | LINC00339 | MTUS2 | LBX1 | RP11-227G15.9 | RP11-78F17.1 |
| RPS27A | OR14C36 | FOXD1-AS1 | CST5 | PLCE1 | CTC-297N7.9 | RP11-691N7.6 |
| MGP | OR4K2 | LINC00892 | C1orf61 | MYPN | RP11-542C16.2 | RP11-254F7.3 |
| DCN | CCL13 | MIR503HG | IGFALS | MSTN | RP11-649A18.12 | RP11-160O5.1 |
| S100A16 | PLPP2 | LINC01551 | RXFP2 | CDK15 | RP11-461A8.1 | RP5-1050D4.3 |
| GAPDH | FEM1A | VIPR1-AS1 | TEKT3 | CHRNA1 | RP11-177N22.3 | RP4-756G23.5 |
| COPZ2 | TMIE | LMLN-AS1 | POSTN | CCDC54 | RP11-210K20.2 | CTD-2325M2.1 |
| CYYR1 | ITGA9 | KRTAP21-1 | HSPB2 | CILP | RP11-344B2.3 | RP11-521C20.3 |
| SEPP1 | ALOXE3 | LINC01124 | CRYBB1 | HCN4 | RP11-705O1.8 | RP11-294N21.2 |
| SAMD14 | BBS12 | LINC00678 | HRASLS2 | PCDH10 | RP11-455O6.2 | RP11-505K9.4 |
| IGFBP4 | TMEM231 | LINC01503 | MRGPRX1 | FGF5 | RP11-143J12.3 | PPAN-P2RY11 |
| RAI14 | SYCE1L | KRTAP5-5 | SLC16A8 | GH2 | RP1-302G2.5 | RP11-660L16.2 |
| APOC1 | IL25 | USP17L15 | HSPB3 | NKX2-8 | RP11-462G12.1 | NPHP3-ACAD11 |
| RPL12 | MAPK15 | MLLT4-AS1 | CNTN6 | MMD2 | RP11-627G18.1 | RP1-67K17.3 |
| UBE2C | RNF186 | KRTAP4-5 | RSPO1 | DGKB | CTD-2012K14.6 | RP11-161M6.2 |
| RBPMS | CYR61 | DCAF12L2 | F9 | CFHR5 | CTD-2026K11.1 | RP11-93B14.6 |
| RPL29 | ALDH1L1 | KRTAP4-1 | CASKIN1 | RAX | RP11-879F14.1 | XXbac-BPG32J3.20 |
| FAM167B | OR3A1 | C1orf147 | RAB26 | GRP | RP11-318A15.7 | RP11-192H23.5 |
| AKAP2 | TMEM52 | FANCD2OS | TEKT1 | LRP4 | CTC-550B14.6 | RP13-580F15.2 |
| CNKSR3 | AXL | POM121L2 | GCM2 | SOX3 | RP11-322E11.6 | RP11-44N21.4 |
| ICA1 | CGN | TMCC1-AS1 | COL21A1 | DSG3 | RP11-105C19.1 | RP11-1100L3.8 |
| ZNF358 | EXOC3L4 | HIST2H4A | ANGPTL4 | DSC3 | RP11-57H14.4 | RP11-212P7.3 |
| RPL13 | OR56B1 | ADAMTS13 | DNAH8 | DSC1 | RP11-126O1.5 | ATP6V0E2-AS1 |
| MTSS1L | KRT6A | CATSPER2 | KLK13 | FHOD3 | RP11-488I20.8 | RPL36A-HNRNPH2 |
| RPL23 | CALML3 | ARHGAP40 | CLDN10 | KDELC1 | RP11-295D22.1 | CTD-2561B21.7 |
| TIMP3 | MYO5B | C16orf71 | LY6D | HRK | RP11-77H9.8 | RP11-290L1.3 |
| PTPN14 | ISLR2 | FAM160A1 | AQP2 | UGT2A3 | RP11-521L9.1 | CTD-3105H18.18 |
| RPS10 | HMCN1 | GOLGA6L10 | ADAMTS8 | UGT2B28 | RP11-449J21.5 | RP11-321N4.5 |
| NR2F1 | VWA5B2 | TNFRSF11B | OR51Q1 | KCP | CTD-2020K17.1 | RP11-397A16.1 |
| MEIS2 | C5orf64 | ARHGAP39 | OR51I1 | EPHA7 | RP11-839G9.1 | RP11-1094M14.12 |
| DDR2 | PCDHGA1 | C17orf100 | MSI1 | CGA | RP11-116D2.1 | NDUFC2-KCTD14 |
| SHC2 | NLRP10 | TP53AIP1 | ART5 | ELF5 | GS1-279B7.1 | CTD-2147F2.1 |
| SNTG2 | HAPLN1 | C11orf52 | PCDH11X | PRPH | RP11-794M8.1 | RP11-483I13.5 |
| RPS16 | PTPRG | LINC01578 | TGM6 | LACRT | CTD-2537I9.13 | CTD-2540B15.11 |
| KRT8 | KLK2 | HEXDC-IT1 | SCG5 | KRT85 | CTC-559E9.5 | RP5-940J5.9 |
| RANBP1 | PSCA | SLC25A48 | NDP | FAIM2 | RP11-546O6.4 | RP11-262H14.4 |
| C1QTNF5 | CACNG8 | ADAMTS19 | MYO1A | MIP | CTC-398G3.6 | RP11-573D15.8 |
| LIFR | KCNN3 | C1orf158 | GNG4 | BCO1 | CTC-548K16.6 | CTC-250I14.3 |
| SEMA3G | CNTN4 | CNTNAP3B | XIRP1 | KCNK1 | AC002398.11 | CTB-43P18.1 |
| CCDC85B | TRIML2 | C11orf53 | TRIM45 | RGS8 | CTC-239J10.1 | RP11-644F5.10 |
| EXOC3L2 | TPRG1 | SPG20-AS1 | RSPO4 | SLC19A3 | RP11-635N19.1 | RP11-649E7.5 |
| BANF1 | AMN | ADIRF-AS1 | IL13 | LECT1 | RP11-255C15.3 | RP5-1113E3.3 |
| HGD | OR56A1 | MAPK8IP1 | FERMT1 | SPDYE1 | RP11-677O4.2 | RP1-78O14.1 |
| FBLIM1 | KLK3 | C14orf79 | PCSK9 | FRAS1 | RP11-352G18.2 | RP11-500M8.7 |
| PLPP3 | NAT16 | LINC00506 | CPT1C | C4orf17 | RP11-567M16.1 | RP11-265N6.1 |
| ROBO4 | MLF1 | LINC00895 | PDYN | SPACA1 | RP5-951N9.2 | RP11-530C5.1 |
| PPARG | ANKRD33 | LINC01232 | TRPM1 | WNT9A | CTC-543D15.3 | RP11-644F5.11 |
| AFAP1L1 | RAB42 | LINC00997 | ROR2 | SLC6A3 | CTD-2588J6.2 | RP11-347C12.3 |
| PTPRB | ERICH3 | GOLGA6L6 | GRM5 | TM4SF5 | RP11-574H6.1 | RP11-792A8.4 |
| ATP5J | TMC4 | ADCYAP1R1 | SLC32A1 | PTH2 | RP11-388M20.2 | RP11-822E23.8 |
| FXYD1 | FAM83H | TMEM151A | ADAM30 | IGLON5 | RP11-517C16.4 | CTD-2396E7.9 |
| CX3CL1 | MMEL1 | HNRNPCL1 | WFDC2 | FHAD1 | RP11-20I23.6 | RP11-96O20.4 |
| ANKRD9 | BHMT | C17orf96 | INSM2 | C1orf94 | RP1-266L20.9 | CTD-2302E22.2 |
| RPL13A | GPR62 | NEXN-AS1 | SHOX2 | SYPL2 | CTD-2008P7.8 | RP11-488C13.7 |
| RBFOX2 | NLGN1 | NAALADL2 | VTCN1 | IGSF3 | RP11-166B2.5 | RP13-131K19.6 |
| TGFB1I1 | GRM2 | C21orf62 | USP26 | FNDC7 | RP11-960L18.1 | RP11-110I1.12 |
| CYP2E1 | MST1R | LINC01558 | CXCL6 | RXRG | RP11-466A19.1 | BHLHE40-AS1 |
| DNPH1 | OR6A2 | C6orf132 | ADAM29 | MAEL | RP11-38G5.4 | TM4SF19-AS1 |
| DGCR6L | LAD1 | LINC00483 | DYNLRB2 | ADCY10 | RP11-114H24.7 | RP11-43N16.4 |
| VPREB3 | STC1 | C1QTNF9B | SMIM17 | F13B | RP4-714D9.5 | RP11-619A14.2 |
| RRM2 | PDZD7 | ADAMTSL1 | CCDC110 | SYT14 | RP11-378A13.1 | RP11-378J18.9 |
| CCDC80 | HHLA3 | KRTAP4-8 | LGI3 | ATP6V1C2 | CTD-3185P2.1 | RP11-362F19.1 |
| RPS7 | OR10H1 | SERPINA12 | KANK4 | MTTP | RP11-96B5.4 | RP11-544I20.2 |
| RPL37A | DNAJC5G | CRTC3-AS1 | HHIPL2 | ABHD1 | CTB-131K11.1 | XXbac-B135H6.15 |
| PODXL | FOXD2 | PDCD1LG2 | FUT3 | C1QL2 | RP5-890E16.4 | CCL15-CCL14 |
| TMEM40 | MC2R | LKAAEAR1 | COQ3 | LYG1 | RP11-37C7.3 | RP11-455O6.8 |
| NUTF2 | NLRP4 | SERPINA3 | CD70 | NXPH2 | CTB-171A8.1 | RP11-629N8.5 |
| GNA11 | FAM86C1 | LINC00639 | SMR3B | CPO | RP11-666A8.9 | RP11-438L7.1 |
| SNCG | RPPH1 | C22orf15 | DLL3 | ABCA12 | RP11-936I5.1 | RP11-103H7.5 |
| RPL4 | CALB2 | MAP1LC3C | OR2K2 | TRPM8 | RP11-127I20.5 | AE000661.37 |
| CEP112 | PCAT19 | FOXN3-AS1 | POU4F3 | CPNE9 | RP11-434D2.3 | EPB41L4A-AS2 |
| MTUS1 | SBSN | RPARP-AS1 | LAMB4 | FAM198A | RP11-165E7.1 | RP1-41C23.2 |
| NQO1 | TIMP4 | CDC42BPG | POPDC3 | SLC22A14 | CTD-2659N19.10 | RP11-706C16.8 |
| MYZAP | OR10H5 | RAMP2-AS1 | ID4 | MYH15 | RP11-24M17.6 | RP11-95F22.1 |
| KIF1C | KBTBD12 | SCOC-AS1 | MAP6 | NR1I2 | RP11-589P10.7 | RP11-216B9.6 |
| RRAS | DUSP8 | LINC00239 | OR10V1 | AGTR1 | RP11-195M16.3 | RP11-253E3.3 |
| SORBS2 | ESM1 | C15orf65 | APOC4 | SPATA16 | CTD-2562J15.4 | RP11-314N14.1 |
| MYEOV | FAM81A | KRTAP1-1 | LRRC8E | CBLC | RP4-657D16.3 | CTD-3184A7.4 |
| AMOTL2 | IL12A | TCTEX1D1 | SSTR4 | DNMT3L | RP11-819C21.1 | SYS1-DBNDD2 |
| FN3K | TMEM17 | C16orf89 | DCDC1 | HUNK | RP11-74C13.4 | RP11-259O2.3 |
| TMEM100 | ABCG4 | SH3PXD2B | THPO | SLCO1C1 | CTD-2369P2.5 | RP11-423H2.3 |
| NUAK1 | GAREML | GOLGA6L2 | SLC9C1 | ANP32D | RP11-7K24.3 | RP1-90J20.8 |
| ZBTB46 | RADIL | PPP1R14C | CPA1 | PTPRQ | RP11-252A24.3 | APOC4-APOC2 |
| RPL11 | OR1S2 | ANKRD13B | OR2AT4 | ASCL1 | RP11-701H16.4 | XXbac-BPG32J3.19 |
| SOX17 | KRT77 | CACNA2D1 | FTHL17 | PDX1 | RP11-434D2.7 | RP11-88H9.2 |
| ADGRL4 | DZIP1L | C19orf57 | CPXM1 | NPFF | RP11-15A1.7 | RP4-622L5.2 |
| MEIS1 | KLHL33 | MAGI2-AS3 | TBX5 | KRT71 | CTC-548K16.2 | LAMTOR5-AS1 |
| CASKIN2 | GREB1 | B4GALNT1 | OTP | RNF113B | RP11-359E10.1 | RP5-877J2.1 |
| SMAD6 | SLC23A1 | CBR3-AS1 | KCNK3 | TTC6 | RP3-466P17.1 | PRKAR2A-AS1 |
| CCND1 | NFASC | ARHGEF16 | NHLH1 | SYT16 | RP11-56B16.4 | RP11-1079K10.4 |
| MPDZ | KRT78 | SERPINC1 | DDC | ADAM21 | RP1-142L7.9 | RP11-1398P2.1 |
| PDE2A | COL27A1 | TOB1-AS1 | REMI | RDH12 | RP11-196H14.3 | RP11-173P15.3 |
| RPL10 | KLHDC9 | BOLA3-AS1 | KRT9 | FAM181A | RP5-999L4.2 | RP11-758P17.3 |
| RAPGEF5 | FAM150A | LINC00710 | SCG2 | SERPINA10 | RP11-256L11.3 | INO80B-WBP1 |
| KIF17 | USP50 | ARHGAP22 | LRRC15 | DUOXA1 | RP11-546B8.6 | GS1-114I9.3 |
| THSD1 | PKDCC | LINC00665 | OR5A2 | DUOXA2 | RP11-395B7.2 | CTD-3074O7.5 |
| C8orf4 | GLDN | GRTP1-AS1 | NEUROD2 | SLC27A2 | RP11-283G6.5 | RP11-428O18.6 |
| PDZRN3 | LRRTM1 | MEIS1-AS2 | APCS | GCNT3 | RP11-88E10.5 | RP11-90J7.3 |
| KIF26A | MMP1 | TNFRSF19 | OR5B2 | CCDC33 | RP11-54G14.1 | AP003419.16 |
| PLPP1 | MUC17 | KIAA1456 | TEKT2 | WDR93 | RP1-34M23.5 | RP11-707G14.1 |
| FHL1 | XIRP2 | ADAMTS17 | HSD17B2 | ABCC12 | RP11-407N8.5 | ADAMTS9-AS1 |
| NEURL1B | PPP1R26 | TMPRSS13 | OR7G2 | MARVELD3 | RP11-87C12.5 | RP11-140L24.4 |
| RPS17 | MSX1 | IGKV3D-20 | TNFSF9 | CHST4 | RP11-444D3.1 | RP11-856F16.2 |
| BAALC | PCDH7 | KIAA1644 | TRH | CLEC18B | RP11-69C13.1 | CTD-2024P10.1 |
| COX5A | NUDT17 | IGLV3-10 | OR10A2 | OR4D1 | RP11-613M10.9 | RP11-229P13.23 |
| MRPL23 | SPRR3 | KRTAP10-5 | ALDH3B2 | SLC25A52 | RP11-804A23.2 | RP11-90P5.2 |
| SAMD4A | DLGAP1 | HOXA11-AS | CEL | SLC13A5 | RP11-427J23.1 | RP11-286N22.6 |
| RPL28 | LAMB3 | C14orf37 | CNGA4 | ZNF750 | RP11-627G23.1 | RP11-486L19.2 |
| MYO10 | SFTPA2 | IGLV3-25 | GNRH2 | ARL5C | RP11-998D10.1 | RP11-382B18.1 |
| YAP1 | CYP3A7 | ARHGEF25 | SEMA4G | ANO3 | RP11-196G11.1 | CTD-2541J13.2 |
| PF4V1 | FAM86B1 | SLC25A47 | MBD3L1 | NGF | CTD-2531D15.4 | RP13-753N3.1 |
| DNLZ | SH3BGR | BRWD1-AS2 | OR10A4 | PEBP4 | RP11-144G7.2 | RP11-805L22.3 |
| CDR2L | PTGER1 | DARS-AS1 | SYT9 | TMEM255A | CH17-302M23.1 | CTC-564N23.2 |
| PLK2 | TPSB2 | C6orf223 | OR8J1 | HOXC11 | RP11-455O6.9 | CTC-455F18.1 |
| RP9 | PGLYRP2 | CRISPLD1 | LINC00868 | DBH | RP11-113D6.6 | RP11-498C9.13 |
| HLF | SCGB3A1 | LINC01160 | OR2AG1 | RAB9B | RP11-736K20.5 | RP11-401P9.5 |
| CRHBP | MUC1 | KRTAP5-4 | MYBPC2 | ESX1 | RP11-569G13.3 | RP11-10A14.4 |
| BCAR1 | CCDC116 | LINC01249 | SLCO1A2 | GPR83 | CTD-2649C14.3 | RP11-1260E13.4 |
| CCL21 | OLFML2A | LINC00905 | TTC36 | MOGAT1 | RP11-687M24.4 | RP11-167N5.5 |
| COL1A1 | SPATA8 | DNASE1L2 | CSDC2 | BTN1A1 | RP11-315O6.2 | RP11-703G6.1 |
| RPL35 | NTF3 | PNLIPRP1 | DEFB126 | SLC17A1 | RP11-1082L8.4 | TMEM256-PLSCR3 |
| CNRIP1 | SYN3 | LINC00534 | FAT2 | SPDEF | RP11-618K13.2 | LLNLR-245B6.1 |
| DAB2IP | ZNF470 | SLC22A11 | FSHR | KLHL31 | RP11-234G16.4 | RP11-383C5.3 |
| UBD | CCDC78 | MCPH1-AS1 | C17orf78 | CRISP1 | CTD-2396E7.11 | RP11-876N24.4 |
| HIGD2A | S1PR2 | KIAA1161 | LINC00599 | SOX21 | RP5-1057I20.6 | CTD-2031P19.5 |
| VAMP5 | OR4C13 | LINC00958 | GLIDR | KIF25 | RP11-727F15.9 | CTD-3137H5.1 |
| ATP5J2 | CFAP126 | LINC00967 | PCDHGB2 | AMELX | RP3-333B15.5 | RP3-422G23.4 |
| SEPT10 | DNAJC5B | HIST1H2AA | NR2E3 | UNC79 | RP11-283I3.2 | RP11-923I11.3 |
| FTH1 | OR5P2 | SEPT7-AS1 | INS | SOX9 | RP11-620J15.4 | CTD-3035K23.3 |
| INMT | PCDH15 | IGKV1D-39 | MIR2052HG | BHLHE23 | LL21NC02-21A1.1 | RP11-14N7.2 |
| TNS2 | C8orf34 | MIR34AHG | FAM230A | GRIA3 | RP11-76I14.1 | RP11-365O16.3 |
| VEGFC | SPRR2E | GMDS-AS1 | EVX1-AS | FOXA2 | RP11-78J21.7 | RP11-305E6.4 |
| PTPN21 | CACNA1C | PSMG3-AS1 | PCDHGA6 | PAX1 | RP13-941N14.1 | RP1-59D14.1 |
| MYL6B | CHST6 | MIR205HG | CSNK2A3 | CST8 | RP1-101A2.1 | RP11-81H14.2 |
| PPFIBP1 | TPBGL | ATP6V1B1 | SMIM18 | NKX2-4 | RP11-597A11.11 | CTD-2561J22.3 |
| VWF | FREM1 | LINC00264 | MAGEL2 | CSTL1 | RP5-864K19.7 | RP11-398K22.12 |
| PHLDB1 | CTNNA3 | TRHDE-AS1 | PCDHGA8 | OVOL2 | RP11-66N24.7 | RP11-805F19.3 |
| TUBB6 | ZDHHC11 | GATA2-AS1 | PABPC4L | FAM83C | RP11-21A7A.2 | RP11-556E13.1 |
| FAM127C | HRASLS5 | C17orf74 | TBC1D3C | KRT36 | RP11-108L7.14 | CTC-429P9.1 |
| HES6 | OR52L1 | KRTAP19-1 | LINC01485 | FLRT1 | RP11-166O4.6 | CTD-2616J11.11 |
| NAP1L1 | TUBB8 | LINC01067 | RDM1 | CTAG2 | RP11-503G7.2 | CTC-360G5.9 |
| KCTD10 | CTHRC1 | FU40194 | PCDHGA5 | NEUROD4 | CTD-2619J13.27 | RP11-539L10.3 |
| PROSER2 | SCHIP1 | PRMT5-AS1 | SLC10A5 | ACTRT1 | RP13-942N8.1 | RP11-244F12.3 |
| VASH1 | KCNJ4 | LINC00861 | TRBV7-7 | BICC1 | RP11-989F5.1 | RP11-294J22.6 |
| MEMO1 | NOS1AP | CNTN4-AS1 | ETV3L | KIAA1549 | CTD-3035K23.7 | RP11-930O11.1 |
| EFEMP1 | HSD3B1 | C11orf16 | LINC01289 | TCF21 | RP11-323P17.2 | RP11-128A17.1 |
| RPL3 | CFAP77 | TSGA10IP | PCDHGA7 | CASQ2 | RP11-294N21.3 | TGIF2-C20orf24 |
| ECSCR | SPRYD4 | LLPH-AS1 | RAD21-AS1 | OLFM3 | RP11-717F1.2 | RP11-203H19.2 |
| PKIG | DPYS | KRTAP8-1 | RHPN1-AS1 | FGF23 | RP11-1038A11.2 | RNF144A-AS1 |
| ZCCHC24 | MGAT5B | TNFAIP8L3 | CKMT2-AS1 | TRIM67 | RP11-77K12.9 | CTC-360G5.6 |
| DYNC1I1 | HPR | GFOD1-AS1 | PLAC4 | BSPRY | CTD-2313J17.6 | RP11-1072A3.3 |
| ECI1 | BTBD17 | KIAA1024 | MIR4458HG | CNNM1 | RP11-277P12.10 | RP11-67L3.5 |
| MYO1C | PRR15 | ANKRD20A1 | BAALC-AS1 | CPN1 | AP003419.11 | RP11-20I23.3 |
| PPP1R3C | PTPN20 | Z83844.1 | SOCS2-AS1 | C10orf95 | RP11-338E21.3 | LA16C-306E5.3 |
| CMTM5 | GJC1 | LINC01088 | ERICD | SFRP5 | RP11-1060J15.4 | AC006547.13 |
| UPC | NEU4 | SERPINA9 | MIR210HG | INSL4 | CTD-2553L13.10 | ATP13A5-AS1 |
| MRAS | KLHL35 | SERPINA6 | LUCAT1 | IFNA6 | RP11-219B17.3 | RP11-366H4.3 |
| CRYAB | TENM4 | LINC00894 | LINC01207 | GRIA2 | RP11-1348G14.8 | RP11-856M7.2 |
| NENF | OR5AN1 | KRTAP13-3 | OXCT1-AS1 | TNN | RP11-831H9.11 | CTD-3193K9.4 |
| SH3D19 | DRD2 | C14orf105 | GABARAPL3 | TTLL2 | CH17-125A10.2 | LLNLR-284B4.2 |
| SEPT4 | ADAM33 | LINC00094 | LINC01095 | TNFSF11 | RP11-236J17.6 | CMB9-22P13.1 |
| NDUFA5 | OSR2 | KRTAP10-9 | GVQW2 | SOHLH2 | RP11-702H23.6 | RP11-97O12.7 |
| SLC8A3 | TAT | RASGEF1C | C5orf17 | PRAMEF2 | RP11-785D18.3 | FAM167A-AS1 |
| ABHD17A | PDE1C | SLC22A12 | PCDHAC1 | ABCC11 | RP5-1160K1.8 | RP11-2C24.4 |
| TRIM2 | TMPRSS2 | PRAMEF17 | LINC01197 | PRB2 | CTD-2562J17.9 | CTB-31O20.6 |
| PTRH1 | SPRED3 | ARF4-AS1 | BACH1-IT1 | LHX4 | RP11-680F20.6 | CTD-3113P16.5 |
| FILIP1L | SKA1 | ANKRD34A | PCP4L1 | CD80 | RP13-631K18.3 | CTD-2369P2.4 |
| EEF1G | PIEZO2 | KRTAP5-10 | TMBIM4 | CPXM2 | RP11-380N8.7 | RP11-17M16.2 |
| ACRBP | ADRB3 | POT1-AS1 | UGT1A7 | PAEP | RP11-622C24.2 | RP11-21L1.1 |
| TTC7B | ARC | BBOX1-AS1 | EHMT1-IT1 | MYOG | CTD-2509G16.2 | RP11-295P9.3 |
| PRX | GPR78 | KRTAP6-3 | LINC00976 | PLG | RP11-327J17.9 | RP11-885N19.6 |
| FABP5 | PPM1J | KRTAP3-1 | DBET | OPN4 | CTD-3128G10.7 | RP11-325F22.2 |
| HRG | SLC29A4 | CEACAM18 | PCBP2-OT1 | SPINK4 | RP4-608O15.3 | RP11-848P1.4 |
| CNFN | ZKSCAN2 | ARHGEF39 | IGKV1D-17 | TAF1L | RP11-467L13.7 | RP13-582O9.6 |
| RBPMS2 | MAGEA8 | C22orf24 | MCCC1-AS1 | RGS13 | CTD-2047H16.5 | RP11-930P14.2 |
| ZNF778 | RPS6KL1 | U91328.21 | OR5V1 | OMD | RP11-850A17.1 | RP11-24N18.1 |
| SATB2 | UXT-AS1 | SLC25A27 | UGT1A4 | TSPAN8 | RP11-802F5.1 | XXbac-BPG181M17.5 |
| AKR1C1 | RAET1E | KIAA1755 | TGFB2-OT1 | EDA2R | RP11-708B6.2 | CTD-2514K5.4 |
| TUBA1B | ZNF334 | C20orf144 | THRU | HIGD1B | RP11-255M2.3 | AC024592.12 |
| GNG12 | TACSTD2 | FAM189A2 | MIR4300HG | CCL25 | CTC-497E21.3 | RP11-1223D19.3 |
| WDR34 | HFE2 | PLEKHG4B | LINC00682 | CDX4 | CTD-2588E21.1 | RP11-152P23.2 |
| ZFPM2 | MRGPRD | ARHGAP36 | PANDAR | KREMEN2 | RP11-672A2.1 | RP11-176N18.2 |
| PDGFB | DNAH2 | HNRNPCL2 | BLACAT1 | CKMT2 | RP11-693J15.6 | RP5-1142A6.8 |
| CLEC4M | BDKRB2 | LINC01565 | RAD21L1 | KRT34 | RP11-148O21.3 | RP11-879F14.2 |
| NDUFAF2 | FAM27C | KIAA1614 | OR2AE1 | KRT33B | RP11-651L5.3 | CTD-2313F11.1 |
| RBP1 | GRM1 | LINC00869 | MIATNB | TNS4 | RP11-734K23.9 | RP11-1151B14.4 |
| RPL18A | ZIC4 | LINC01588 | LINC00934 | LRRC9 | CTC-276P9.4 | RP11-529K1.2 |
| MEA1 | ASPHD1 | SLC26A10 | SCHLAP1 | MATN3 | RP11-434E6.4 | RP11-99J16_A.2 |
| A1BG | March11 | HS3ST3A1 | LINC01395 | SLC6A11 | RP11-672A2.4 | CTD-2666L21.1 |
| PLCD3 | LGI4 | LINC01420 | LINC00461 | LINC00470 | RP11-697E22.2 | RP11-432B6.3 |
| KALRN | FZD4 | HIST1H2AJ | CTBP1-AS | SLX1A | RP11-535A19.1 | RP11-56L13.3 |
| PRTN3 | MOBP | HIST1H4B | EPHA5-AS1 | HHLA1 | RP11-496I9.1 | AC006946.15 |
| EFHD1 | SPRR2D | ARHGAP23 | ZFPM2-AS1 | IQCA1 | RP11-486I11.2 | CTB-25B13.12 |
| CDT1 | JPH3 | HIST1H3I | C8orf17 | ENAM | RP11-203M5.2 | RP11-304L19.13 |
| RPS23 | FOXL2 | KIAA1217 | LINC00491 | SLC52A1 | RP11-326C3.2 | CTD-2026K11.4 |
| LAGE3 | MFSD4 | DNASE1L3 | WWC2-AS2 | RGS22 | RP11-677M14.2 | RP11-4L24.3 |
| TMEM54 | OLFML1 | SLC9A3R2 | MIR3945HG | RAB25 | CTD-2012K14.8 | RP11-661A12.7 |
| FLT1 | PODN | C1orf115 | RFPL4B | LPIN3 | RP11-624G17.3 | RP11-317N8.5 |
| NRSN2 | OR2Y1 | HIST1H2AI | ALG1L2 | DMGDH | RP11-50I19.2 | RP11-350G8.9 |
| CENPB | VASH2 | HIST1H3F | LINC01234 | PDE6A | RP11-160H12.2 | RP1-178F15.4 |
| AMOTL1 | ADCYAP1 | HIST2H3D | MIR143HG | CCNA1 | RP3-403A15.5 | RP11-701B16.2 |
| CAV2 | CCL22 | HIST1H2AB | FER1L5 | TRPC4 | RP11-201M22.1 | RP11-322E11.2 |
| ZNF423 | FGD1 | HIST1H4D | LINC01126 | STOML3 | RP11-411B6.6 | RP11-793H13.3 |
| TRIP10 | LIPG | HIST1H4L | LINC00939 | GSTT2B | RP11-700F16.3 | CTB-186H2.3 |
| ZNF205 | HAPLN2 | C12orf57 | LINC00964 | GGT2 | RP11-378E13.3 | RP11-977G19.11 |
| PRKCDBP | PSG3 | LINC00657 | HOXC-AS2 | SFTPD | RP11-567I13.1 | RP11-521B24.5 |
| IGFBP2 | PIGY | KIAA1462 | C20orf181 | PYY | RP11-148O21.2 | RP1-253P7.4 |
| NPR1 | CELF4 | ARHGEF15 | VCAN-AS1 | ULBP3 | RP11-358L22.3 | RP5-956O18.3 |
| IFI27 | ZBED3 | C19orf33 | MGC32805 | TAS2R4 | RP11-732A19.5 | RP11-463J10.3 |
| C1QC | PROKR2 | PPP1R14A | LINC00216 | RGN | RP11-90L1.8 | RP11-469H8.8 |
| EMCN | CHRM3 | HIST1H3J | HS3ST5 | TRPV5 | CTD-2523D13.1 | RP11-96K19.5 |
| HOXB7 | DOK5 | HIST1H3C | GOLGA8K | OR7A10 | RP11-297N6.4 | LL22NC03-75H12.2 |
| TCEAL3 | TBC1D8B | HIST1H3E | GOLGA6L22 | OR7C2 | RP11-478K15.7 | GS1-114I9.1 |
| E2F1 | NFATC4 | HIST1H2BB | FENDRR | OR7C1 | CH17-360D5.2 | LL22NC03-63E9.3 |
| F2RL3 | MYO18B | PPP1R14B | FAM156A | WFIKKN1 | RP11-290H9.5 | RP11-14J7.6 |
| TMA7 | ISM2 | C17orf89 | SPDYE6 | IL17B | WI2-8325B5.1 | RP11-101E13.5 |
| ALDH7A1 | CHL1 | MMP24-AS1 | ARL14EPL | SPINK2 | RP11-123K3.9 | RP1-317E23.6 |
| ACE | PSG7 | ITGA9-AS1 | CCDC187 | RFPL3 | RP11-495O11.1 | RP3-508I15.21 |
| CXCL3 | ESRP2 | SH3PXD2A | GABRQ | APOL5 | RP11-5L12.1 | RP11-182J1.3 |
| FAM219A | DLX5 | ARHGAP29 | PTOV1-AS1 | BBOX1 | RP1-102E24.10 | RP5-874C20.8 |
| CISD3 | TFPI2 | ANKRD33B | LYPD8 | KCNC1 | RP11-121C6.5 | CTD-2517M22.14 |
| FN1 | HAMP | HIST1H2BK | SCGB1B2P | FOXJ1 | RP11-468E2.10 | RP11-219B4.5 |
| CDC20 | FAM83E | SLC25A53 | LINC00664 | ART1 | RP11-407G23.7 | RP11-111M22.3 |
| RHOJ | CRX | SLC25A15 | LCMT1-AS1 | INS-IGF2 | AP001505.10 | RP11-70F11.8 |
| APOC3 | GFPT2 | C12orf75 | KCNJ18 | GALNT8 | RP11-400G3.5 | RP11-74E22.8 |
| MEIS3 | TLE6 | PRICKLE2 | MIR497HG | FAM155B | CTD-2609K8.3 | RP11-375N15.2 |
| RPL5 | IL4I1 | C11orf96 | HEXA-AS1 | TSPAN16 | RP11-258O13.1 | AC009133.21 |
| RPS15 | CALB1 | CDC42EP5 | SMIM22 | C19orf80 | RP11-116D17.4 | RP11-731F5.1 |
| GPR4 | TRPA1 | HIST1H3B | IGKV1D-43 | LRCH2 | RP11-727A23.10 | RP11-731D1.4 |
| RPS5 | HMGCS2 | ARHGAP28 | ERVK-28 | ACSBG2 | RP5-1165K10.2 | RP4-559A3.7 |
| LY6G6F | WNT2B | HIST1H2AL | RNF225 | FUT5 | RP4-539M6.22 | C1QTNF9B-AS1 |
| CTTN | REEP1 | HIST1H2BM | MGC15885 | BMP15 | CTD-2555A7.2 | RP11-701H24.8 |
| RPS2 | MT3 | HIST1H2BH | LINC01169 | CACNG6 | RP11-944C7.1 | HORMAD2-AS1 |
| FKBP10 | CPEB1 | C16orf13 | TMC3-AS1 | SULT4A1 | RP11-293M10.1 | CTC-203F4.2 |
| RAI2 | DUSP13 | HIST1H2AH | LINC01235 | CALY | GS1-72M22.1 | LA16C-358B7.4 |
| EVA1B | MYH7B | C22orf46 | PLCB2-AS1 | PRDM12 | RP11-350N15.6 | RP11-178L8.8 |
| UNC13B | PAK3 | CTNNBIP1 | UBE2Q2L | FIBCD1 | RP1-286D6.5 | CTC-543D15.8 |
| CDCA5 | RGS11 | OIP5-AS1 | CALR3 | PKDREJ | RP11-155O18.6 | RP11-566K19.6 |
| RPL18 | SEMA3A | HIST1H4F | TRABD2B | NUTM2F | RP4-665J23.4 | RP11-324E6.6 |
| CD24 | TACR2 | LINC01011 | PCSK6-AS1 | NLRP8 | RP11-348P10.2 | CTD-2184D3.6 |
| PDLIM3 | SNCB | C16orf45 | Z69720.2 | ATOH7 | RP13-379O24.3 | RP11-598F7.5 |
| CLEC4G | PPP2R2C | TMEM178A | BLID | IGKV2D-28 | RP11-661G16.2 | RP11-203B9.4 |
| TINAGL1 | TP63 | LINC01140 | FAM215A | LINC01268 | RP11-1152H15.1 | RP11-512M8.5 |
| ITGA2B | MOV10L1 | KIAA1211 | MIR1539 | LINC01359 | CTD-3051D23.1 | HSPB2-C11orf52 |
| PTPRF | FRMPD1 | MBNL1-AS1 | LINC00165 | RBMY1J | RP11-480D4.6 | RP11-867G23.4 |
| RPL27 | EFR3B | HIST1H4J | LINC00922 | C17orf112 | RP11-471M2.3 | RP11-212D19.4 |
| TUBG1 | PTHLH | ARHGEF28 | SEPT4-AS1 | ITGB2-AS1 | RP11-109N23.4 | RP11-380O24.1 |
| GNB2L1 | CRB1 | HSD17B14 | MAPT-AS1 | SPANXB1 | RP11-293M10.2 | RP11-182J1.1 |
| IL33 | GCGR | LINC00923 | TMEM249 | DLG1-AS1 | CTD-2014B16.3 | RP13-514E23.2 |
| MCF2L2 | SLC7A4 | HSD11B1L | LINC00668 | NCAM1-AS1 | RP11-481J13.1 | RP11-398H6.1 |
| HOMER3 | CECR2 | IGKV3-15 | ZNF488 | FLJ43879 | RP11-97O12.6 | RP11-804A23.4 |
| RPS6 | DSG1 | LINC01554 | PCDHGA4 | C1orf234 | CTD-2342N23.3 | RP11-569G13.2 |
| BAD | GABRP | TMEM150C | MIA | SRRM5 | RP11-638I2.6 | RP11-248E9.6 |
| VAMP3 | TYRO3 | HLA-DQB2 | KLF14 | DAB1-AS1 | RP11-72M17.1 | RP11-713P17.3 |
| NRGN | CCL3L3 | TMEM255B | LCN6 | SMCR5 | FAM181A-AS1 | CTD-2562J17.2 |
| AKR1C2 | HTR1B | NDUFA4L2 | ADPGK-AS1 | CADM3-AS1 | RP5-1039K5.17 | CTD-2653D5.1 |
| GP1BB | IFNA4 | C16orf59 | WDR7-OT1 | DBH-AS1 | RP11-713M15.2 | RP11-989E6.10 |
| CCDC3 | CD209 | KIAA1522 | LINC01541 | P3H2-AS1 | RP11-474O21.5 | RP11-454K7.1 |
| FSCN1 | CHRD | LINC01151 | LINC00543 | TRAM2-AS1 | RP11-444E17.6 | RP11-571M6.15 |
| MRPL12 | SLC4A11 | LINC00504 | LINC01415 | UBXN7-AS1 | RP11-157F20.3 | RP11-796E2.4 |
| SWI5 | DFNA5 | TMPO-AS1 | KC6 | LINC00694 | RP11-762H8.4 | CTD-2049O4.1 |
| TEAD2 | COL5A1 | NPPA-AS1 | DSG1-AS1 | FRG2B | RP11-284F21.10 | RP11-770G2.5 |
| HOXD8 | LYPD3 | IGLV2-11 | LINC01443 | CTAGE4 | RP11-192P3.4 | RP11-566K11.5 |
| YES1 | TSPAN1 | WDR11-AS1 | GDF10 | NRIR | RP11-950C14.3 | RP3-329E20.2 |
| ARMCX6 | ARTN | TCEB3-AS1 | WFDC21P | OR52I2 | RP11-545M17.1 | CTC-338M12.7 |
| CABLES1 | GRM4 | LINC00152 | LINC01228 | NUP50-AS1 | RP11-509A17.3 | ARHGEF26-AS1 |
| SPHK1 | KCNQ4 | RASGEF1A | TRIL | LINC01535 | RP1-40E16.12 | RP11-658F2.8 |
| LNX1 | HAO2 | SMIM10L1 | OR4D2 | LENG8-AS1 | RP11-433J8.1 | RP11-637A17.2 |
| RPS3 | SLC17A3 | LINC01116 | LINC00346 | BOD1L2 | RP11-218E20.3 | RP11-598F7.6 |
| ATP5G2 | MOCS1 | C11orf95 | KRTAP7-1 | IGKV6D-21 | RP11-214N1.1 | RP11-59N23.3 |
| ZNF462 | KCNK17 | FU22447 | HNF1B | SPINK8 | RP11-204N11.1 | CTC-441N14.2 |
| DOC2B | HULC | SERPINA5 | CCL18 | PGA3 | RP11-138I18.2 | RP11-116G8.5 |
| FARP1 | OR6V1 | SLC16A11 | C7orf77 | FU31356 | RP11-307C12.12 | RP11-706O15.5 |
| LSM2 | DLX2 | NAPA-AS1 | TCEB3CL2 | TRPM2-AS | RP11-435O5.6 | RP11-674E16.4 |
| MSANTD3 | PROC | PRC1-AS1 | PAPPA-AS1 | PRRT3-AS1 | RP11-407N17.4 | CTD-2545H1.2 |
| TRIM47 | EVA1A | ZBED5-AS1 | OR13A1 | C9orf147 | RP11-1070N10.6 | RP11-386I8.6 |
| SLCO2A1 | March2 | C1orf226 | LINC00273 | NFE4 | RP11-1112J20.2 | RP11-680F8.1 |
| TMEM230 | COLEC11 | OR2A1-AS1 | LIMS3L | PROX1-AS1 | RP3-523E19.2 | RP11-111H13.1 |
| EIF3F | GHSR | RAB30-AS1 | STH | USP17L1 | RP11-280K24.1 | RP11-216L13.16 |
| CKS1B | TRNP1 | MARVELD2 | SMIM11B | LINC00452 | RP11-171I2.4 | RP11-1134I14.8 |
| PTGES3L | TBX4 | LINC00649 | TPTE | RFPL4AL1 | RP11-342K6.4 | RP11-538I12.2 |
| GCOM1 | NXPH1 | SIGLEC11 | GOLGA6L1 | C1orf143 | RP5-1057J7.7 | RP11-1149O23.3 |
| PLEKHA6 | KCNJ5 | OR2F2 | ASIC5 | LINC01320 | RP11-502I4.3 | RP11-327F22.5 |
| NR1H3 | SEC16B | HEPN1 | DND1 | IGKV3D-7 | RP11-379K22.3 | AC015691.13 |
| ACOT7 | LRP11 | OR2A25 | SALL3 | ZNF883 | RP11-1085N6.3 | RP11-27K13.3 |
| RBMS3 | ITIH5 | KHDRBS3 | C18orf65 | TTC3-AS1 | RP11-2E11.6 | RP11-867O8.5 |
| C4orf48 | CRHR1 | OR6N1 | FAM74A7 | SAPCD1 | RP11-342K6.1 | RP11-603J24.9 |
| PSD3 | KCNIP2 | CTF1 | GTSE1-AS1 | UBAC2-AS1 | RP11-379K22.2 | LA16C-313D11.12 |
| TACC2 | ESRRB | IP6K3 | LINC00235 | SATB1-AS1 | RP11-14J7.7 | ST3GAL6-AS1 |
| TSPAN9 | WFDC3 | MPP3 | OR5M1 | GAS8-AS1 | RP11-85G21.3 | RP11-613M10.8 |
| ADH4 | EDN3 | REG3G | PGM5P2 | LINC01356 | RP3-337O18.9 | RP11-676J12.7 |
| RPS24 | TLX2 | NRK | NPIPA2 | LACTBL1 | RP11-131H24.4 | RP11-273B20.1 |
| NT5M | ZNF229 | NTNG1 | OR13C5 | TTC34 | RP11-270M14.5 | RP13-820C6.2 |
| PTPN11 | STEAP3 | CYS1 | OR5M10 | SP9 | RP11-428J1.4 | RP11-659E9.2 |
| WSCD1 | SMO | IL20 | RSF1-IT1 | SYCE3 | AC007040.11 | RP11-175K6.1 |
| FMO2 | WNT5B | SNHG23 | ANKRD20A3 | PAPOLB | CTD-2287O16.5 | CTD-2354A18.1 |
| EHD3 | PTPN5 | CYP2A6 | NOX5 | TEX40 | RP11-1017G21.4 | RP11-148O21.4 |
| RPL38 | MEX3A | FOXN4 | OR8G5 | HTR5A-AS1 | RP11-482H16.1 | RP13-638C3.4 |
| RPL36 | HPX | TICRR | SPATA31A7 | IGBP1-AS2 | RP11-934B9.3 | CTD-2373J6.2 |
| PKP4 | SNHG14 | FAM222A | PRSS2 | EBF2 | RP11-436D23.1 | RP11-783K16.10 |
| DOCK6 | FAM64A | PLAU | PADI6 | KRTAP1-5 | CTD-2062F14.3 | CTD-2006C1.10 |
| RPS20 | HCG25 | RASL10B | TRBV13 | POTEJ | RP11-47I22.4 | RP1-29C18.9 |
| ZNF366 | DPP6 | FAM71E1 | CASC15 | OR2A12 | RP11-356B19.11 | RP11-379F4.4 |
| C1QA | ATRNL1 | BARHL2 | MC1R | KRTAP12-2 | CTD-2235C13.3 | RP11-809N8.2 |
| IL34 | CCDC136 | CNIH3 | C20orf141 | KRTAP1-3 | RP11-426L16.10 | RP11-367J11.3 |
| RDX | MYL2 | NT5DC4 | LINC-ROR | HMSD | AE000662.92 | KB-1471A8.1 |
| THBS1 | KRT17 | MYLK3 | U91328.19 | OR1C1 | CTD-2201E18.5 | RP11-867G23.8 |
| LRRC32 | PRRT4 | VGLL3 | LINC00640 | NPTXR | RP11-1017G21.5 | AC002310.11 |
| FABP1 | GHR | AKR1D1 | SNHG24 | OR6X1 | RP1-202O8.3 | RP11-353N14.4 |
| CARD10 | WNK4 | CCDC155 | LPP-AS2 | XKR9 | TVP23C-CDRT4 | RP11-342D11.2 |
| GPRC5B | C7 | SH3GL3 | ADAM20P1 | C3orf36 | RP11-187E13.1 | CTD-2547L16.1 |
| FAM213A | PDE10A | TRAV41 | C17orf50 | OR2A2 | CTA-223H9.9 | RP11-473I1.6 |
| BTNL9 | CYP21A2 | SNHG17 | DUXA | PSMB11 | RP11-458F8.4 | RP11-116D17.1 |
| CDH5 | HPCA | TSSK1B | LINC01215 | CELA2B | RP11-780K2.1 | RP11-620J15.3 |
| FABP3 | TRAV16 | HCN3 | DIO3OS | HMX1 | RP5-1039K5.18 | COL18A1-AS1 |
| NOVA2 | STYK1 | CYP1A1 | KRT73-AS1 | C20orf202 | CTD-3148I10.15 | RP11-463O12.3 |
| PRADC1 | CYP11A1 | VSNL1 | KB-7G2.8 | GAB4 | RP11-434H6.6 | RP11-108M9.4 |
| TSPAN12 | OR10G2 | TCP11 | LYPD4 | FAM209B | RP11-114F10.3 | RP11-69L16.4 |
| FERMT2 | IYD | MUC19 | ZBTB8B | ZNF888 | AC000095.11 | RP11-14D22.2 |
| ADGRF5 | ZNF112 | TRAV8-3 | RBAKDN | ZNF99 | RP3-521E19.2 | RP13-1032I1.7 |
| CD5L | BCL2L10 | OR2B6 | GAS6-AS2 | C12orf74 | RP11-290D2.6 | RP11-414J4.2 |
| DNAAF3 | FMO3 | ITIH3 | LINC00935 | SPDYE3 | RP11-144F15.1 | RP11-264B17.5 |
| BOLA3 | SLC6A16 | UNC5CL | HIST1H4K | FOXI3 | XXbac-B444P24.14 | RP11-758P17.2 |
| TPO | NTRK3 | MATN1 | GRK1 | NOTO | RP11-416N2.4 | RP11-867G23.3 |
| COL4A2 | ATP2C2 | SCN8A | STPG2 | IQCF6 | RP11-84G21.1 | CTD-2033A16.1 |
| PDGFRA | ITPKA | FAM84A | LRRC53 | CDIPT-AS1 | RP11-78I14.1 | RP11-806J6.1 |
| CKS2 | USH1C | PPFIA2 | AKNAD1 | MS4A18 | RP11-201K10.3 | RP11-293A21.1 |
| FAM69B | CSH2 | HS3ST6 | HFM1 | RAD51AP2 | RP11-385F5.5 | RP11-586K2.1 |
| RSC1A1 | LRRC30 | KCNK15 | BRINP3 | LINC00612 | RP11-1103G16.1 | RP1-43E13.2 |
| NXN | TMPRSS4 | TEKT4 | KCNJ9 | DCDC2C | RP11-579D7.2 | PPP1R26-AS1 |
| NTN4 | ETV1 | MT1M | C1orf74 | NPIPA7 | RP11-1246C19.1 | CTC-338M12.6 |
| ZNF704 | PRSS3 | LMAN1L | FAM71A | GSG1L2 | RP11-571M6.7 | RP11-864G5.3 |
| EML1 | NGEF | PDHA2 | AXDND1 | COL28A1 | RP11-834C11.14 | RP11-297B17.3 |
| MAGIX | CYP46A1 | IRGC | TDRD5 | GRXCR1 | RP11-474G23.1 | RP11-550A5.2 |
| GJA4 | CPS1 | RAB17 | WDR64 | UBE2QL1 | RP3-412A9.16 | RP11-508N12.2 |
| PPIC | ATP1A2 | BBS5 | FCAMR | ZNF705D | RP11-993B23.3 | CTD-2651B20.7 |
| SPRY1 | FGF2 | COL2A1 | FRZB | ZNF705B | AE000658.22 | RP11-834C11.4 |
| TMEM44 | GSTA2 | SSTR5 | SPATA18 | ZNF705G | RP11-22N19.2 | RP11-71G12.1 |
| ZNF608 | RAD51 | CAPN13 | BIRC8 | KRTAP10-4 | RP11-425A6.5 | RP11-290H9.2 |
| MXRA7 | CHDH | TWIST1 | TMOD4 | AATBC | RP11-531F16.4 | CTC-558O2.2 |
| IRX3 | SEMA3B | DQX1 | TGM4 | FAM230B | RP11-545P7.4 | RP11-76E12.1 |
| AP3S1 | BISPR | FAM180B | LCE3D | EFCAB8 | RP11-357H14.16 | RP11-582J16.3 |
| PALD1 | SLCO2B1 | LRRC52 | BMP10 | C10orf55 | HOXA10-HOXA9 | RP11-37B2.1 |
| SULF1 | TRO | PNMT | CRYGC | RFPL1S | RP11-689B22.2 | RP11-70C1.1 |
| C8orf82 | ADAD2 | NYNRIN | ALPPL2 | THRB-IT1 | RP11-848D3.5 | RP11-182J1.13 |
| PREX2 | MPPED2 | CTAGE1 | CLDN1 | OPA1-AS1 | RP1-74B13.2 | CTD-2035E11.3 |
| DKK3 | TFAP2A | STAC2 | C3orf30 | U82695.9 | RP11-298I3.1 | CTD-2270L9.2 |
| ELN | SOX8 | ZNF774 | TMEM169 | CATIP-AS1 | RP1-100J12.1 | CTD-2012K14.3 |
| COL1A2 | ACSM2B | PTH2R | TSACC | LINC00337 | RP3-402G11.26 | RP11-152H18.3 |
| ARNTL2 | CDCP1 | ADRA2B | IHH | IGLV9-49 | LL09NC01-251B2.3 | RP11-227H15.5 |
| HSPG2 | PSG1 | ZNF165 | RETNLB | MIR137HG | RP11-834C11.12 | RP11-23P13.4 |
| ADAMTS9 | FZD10 | C4BPB | FCRL4 | CT62 | RP11-341G23.4 | RP5-972B16.2 |
| IL17RC | KLK4 | TIGD7 | SLC2A2 | NRSN2-AS1 | RP13-270P17.3 | RP11-566K11.4 |
| CALCRL | CLDN25 | COX6B2 | NKX6-1 | SFTA1P | RP11-887P2.6 | CTD-2600O9.1 |
| ACER2 | KLK11 | BMP2 | CCDC158 | NHEG1 | RP11-484K9.4 | RP5-1086L22.1 |
| ERRFI1 | EDDM3A | SPTBN4 | LRRIQ3 | RFPL4A | RP11-864J10.4 | RP3-475N16.1 |
| ACVRL1 | AICDA | RHBDL3 | CCDC27 | OSER1-AS1 | RP3-428L16.2 | RP1-34H18.1 |
| MEOX2 | KLK6 | PROK1 | LRRC38 | ROR1-AS1 | RP11-588H23.3 | RP1-240B8.3 |
| SHE | FAM9C | FTCD | TMEM82 | IGKV3D-15 | RP11-136F16.1 | RP11-315D16.2 |
| KITLG | KRT80 | GADL1 | EDA | EGFR-AS1 | RP11-725P16.2 | RP11-728F11.4 |
| CXCL11 | SKIDA1 | CYP2C8 | WNT9B | LINC00466 | RP11-1028N23.2 | RP11-371E8.4 |
| SNAI2 | GSG1 | ITGA10 | ACTC1 | CHL1-AS2 | RP11-259N19.1 | RP3-513G18.2 |
| TBC1D16 | GPRC5D | FBXL22 | SIM2 | C4B | RP11-293I14.2 | XXbac-BPG154L12.4 |
| AMBP | DAND5 | F10 | LCE2B | LINC00365 | CTD-2026G6.3 | RP1-122O8.7 |
| SNHG19 | OR5I1 | LRRC46 | PGLYRP3 | LINC00885 | RP4-816N1.6 | RP11-395B7.4 |
| OIT3 | ADRA1A | CROCC2 | AGRP | ZNF812 | RP11-339B21.13 | RP11-342M1.3 |
| DENND2A | OR8A1 | CFAP157 | ZBTB8A | FTX | RP11-536C10.16 | RP3-508I15.9 |
| RBP4 | COL12A1 | TAS2R38 | TFF2 | LINC00851 | RP4-647C14.2 | AC017101.10 |
| ECM1 | EDDM3B | KNCN | CBS | LINC01563 | RP11-479O9.4 | RP11-1069G10.1 |
| EGLN3 | MDFI | SLC35G6 | HSF2BP | BAALC-AS2 | RP11-173D3.1 | RP11-624L4.1 |
| RND3 | SBK2 | GPR17 | LRRC3 | EHMT2-AS1 | RP11-384J4.2 | RP11-798K3.2 |
| ATP5EP2 | PCDHB3 | INAFM2 | TMEM190 | TAAR9 | BCDIN3D-AS1 | RP11-318C24.2 |
| OPHN1 | GRM8 | PRR22 | PTH1R | PATE4 | RP11-170N16.3 | RP11-519G16.5 |
| PDE7B | GJB4 | MDGA2 | LRRC71 | C3orf79 | RP11-190C22.9 | RP11-150O12.6 |
| ASAP3 | OR9A2 | LY6K | COL26A1 | HOXD-AS2 | RP13-616I3.1 | CTD-2292P10.2 |
| C1QTNF1 | PRRX2 | CADM3 | CCDC105 | LINC00959 | RP11-517H2.6 | RP11-258F22.1 |
| MAP1B | DNALI1 | TMEFF2 | USP41 | OR7E24 | RP11-647K16.1 | RP5-1111A8.3 |
| SNX7 | RBFOX3 | SLAMF9 | KLHL10 | FRY-AS1 | RP11-596D21.1 | CTD-2192J16.17 |
| FAM207A | ATP13A5 | OR2M5 | ASB16 | PRKG1-AS1 | KB-1125A3.11 | RP11-107I14.4 |
| KRT19 | CDHR4 | MATN4 | SHANK1 | RC3H1-IT1 | RP11-290L1.2 | RP11-1078H9.5 |
| LAMP5 | GPRC5A | WIPF3 | AQP5 | LINC00896 | CTD-2591A6.2 | RP11-108M9.3 |
| FGF12 | PERP | CYP3A4 | OR7G1 | LINC01055 | RP11-497G19.1 | RP11-360L9.7 |
| HES1 | LPO | OR8D1 | KRT82 | RPEL1 | CMP21-97G8.2 | RP11-317G6.1 |
| PCP2 | KCNJ11 | LRRC24 | SYCE2 | HIPK1-AS1 | RP5-1103G7.10 | RP1-117B12.4 |
| SPIC | OR4X2 | DPT | CCDC42 | BARX1-AS1 | RP5-943J3.2 | RP4-813D12.3 |
| DMKN | TTYH1 | KIF26B | CCDC64B | NKX1-1 | CTB-152G17.6 | RP11-242F24.1 |
| TANC1 | OR52D1 | OR8B2 | FAM151A | RNF148 | RP11-265E18.1 | GS1-124K5.11 |
| KIF20A | CYP27B1 | C3orf84 | BSND | LINC00298 | RP11-25E2.1 | RP11-565P22.6 |
| PINLYP | PPY | SLC35F3 | TCF23 | LINC01553 | RP1-228P16.4 | CTD-2527I21.4 |
| LYPD5 | ADAM18 | UTS2R | SLC6A20 | OR5H14 | RP5-1159O4.1 | RP11-262H14.3 |
| EPB41L1 | OTC | OR10G9 | ITLN2 | LINC01376 | RP11-210M15.1 | RP11-798M19.3 |
| JAM2 | RTN4RL2 | FAM162B | SOHLH1 | LINC01361 | CTD-2021H9.3 | WI2-2373I1.2 |
| NBL1 | ALDH3A1 | C2CD4B | TMC1 | USP12-AS2 | AP001432.14 | AC009133.17 |
| SEMA3F | ASPA | OR52J3 | SSMEM1 | UGT1A1 | LINC00266-1 | RP11-90C4.1 |
| TOMM40 | C1QL4 | ANKRD45 | CFAP47 | OR14A2 | RP11-276H1.3 | RP11-495K9.3 |
| APOL4 | ARSJ | NPBWR1 | CXorf58 | LINC00881 | RP5-856G1.2 | RP11-124O11.1 |
| ALDH1A3 | TRIM73 | XKRX | PTCHD1 | KRTAP5-8 | RP11-334J6.7 | RP4-598P13.1 |
| TF | CSF3 | ARSI | GPR101 | WWTR1-AS1 | CTD-2550O8.5 | PRR5-ARHGAP8 |
| STOX2 | SORCS1 | CDR1 | CLDN2 | HNF1A-AS1 | RP5-1187M17.10 | C8orf34-AS1 |
| NNT-AS1 | CTRB2 | C5orf49 | LRFN5 | PAQR9-AS1 | XXbac-B461K10.4 | RP11-264E18.1 |
| C1QB | KAZALD1 | ACOT1 | MBL2 | TMEFF1 | ZNF670-ZNF695 | RP11-802E16.3 |
| TNNC2 | USP2 | PRSS58 | GJB2 | HOGA1 | CTD-3222D19.2 | RP11-354E11.2 |
| PARVA | TDGF1 | FAM166B | SSX5 | KIR3DL3 | DKFZP434K028 | RP11-310H4.2 |
| PRRG1 | APBA1 | GMNC | OTX2 | LINC01100 | RP11-51F16.8 | RP3-460G2.2 |
| PLCB4 | KRT16 | MUC5AC | OXGR1 | OR51B5 | RP11-178L8.4 | RP11-290F5.1 |
| PRR7 | TYRP1 | SAMD13 | UCMA | TCP10L | LA16C-395F10.1 | RNASEH1-AS1 |
| FGB | FOXI2 | ARHGDIG | PPP1R36 | PSG2 | RP11-166N17.1 | LL0XNC01-250H12.3 |
| FGG | OR4C15 | RBM20 | RTP3 | BGLAP | XX-C283C717.1 | RP11-152F13.10 |
| SYT17 | GCK | GDF3 | HSPA12A | LINC00996 | RP13-188A5.1 | RP11-211G23.2 |
| CETP | TLL1 | PLPP4 | OTOGL | PCDHGC4 | RP5-884G6.2 | RP11-335O4.3 |
| FBLN1 | HPDL | BHLHE22 | SLC6A5 | LINC01206 | RP11-152N13.5 | RP11-967K21.1 |
| SHANK2 | CADPS | RNASE13 | MYRFL | PCDHGC5 | RP11-796I2.1 | TMEM161B-AS1 |
| CHST8 | VN1R2 | FOXL2NB | CCDC182 | PRKCQ-AS1 | AC087499.10 | RP11-161H23.9 |
| LAPTM4B | IL23A | OR56B4 | OR2D2 | LINC01410 | RP11-418J17.3 | CTD-2314B22.1 |
| PLAT | ECSCR | FAM74A1 | SERPINB7 | LINC00426 | RP4-565E6.1 | CTB-127C13.1 |
| CADM2 | OR1F1 | KCNQ3 | IQCD | BRWD1-AS1 | RP11-536C5.7 | RP13-317D12.3 |
| SSUH2 | APOA4 | DIO2 | CFAP52 | CD81-AS1 | FAM212B-AS1 | FXYD6-FXYD2 |
| BEX1 | CNTN1 | PGA5 | SERPINB12 | NCK1-AS1 | RP11-103C16.2 | MSH5-SAPCD1 |
| HSPB8 | OR10K2 | NPB | CHRFAM7A | POM121L7 | CTD-2330K9.3 | RP11-256I9.2 |
| LRRC49 | CPZ | TECRL | MMP10 | MYLK-AS1 | RP11-350G8.5 | RP1-32I10.10 |
| APOA2 | ACTL9 | PENK | TMPRSS5 | PRAMEF11 | RAPGEF4-AS1 | RP4-639F20.1 |
| CHRNA4 | KCNG4 | GDF5OS | HTR3A | IGKV1-6 | LYPLAL1-AS1 | AC002310.12 |
| RASAL2 | PHYHIP | SDR42E2 | PRKACG | KRTAP9-2 | RP11-212D3.4 | TRIM39-RPP21 |
| EMILIN1 | TPRX1 | C8orf49 | CA3 | GHRLOS | RP11-269F19.2 | RP11-110I1.6 |
| B4GALT2 | BCL2L14 | C1orf64 | TMEM74 | LCE1F | RP1-234P15.4 | RP11-650J17.1 |
| METTL1 | CHP2 | IGKV5-2 | DEFA5 | LINC00636 | RP4-575N6.4 | RP11-973D8.4 |
| PAK6 | TEX13A | SYT15 | KLF15 | C1QTNF9 | RP1-69D17.4 | RP11-108010.8 |
| COL6A1 | ADCY1 | TCEAL6 | CFAP100 | KRBOX1 | RP1-137D17.1 | KIRREL3-AS3 |
| GSTM3 | SLC9A4 | GLT6D1 | FBXW12 | MCF2L-AS1 | RP11-536O18.1 | RP11-287D1.4 |
| TMEM108 | ASB4 | OR4F6 | PRSS12 | IDH1-AS1 | RP4-760C5.5 | RP5-1097F14.3 |
| GSTM5 | HOXB1 | GPRIN2 | NDST3 | MAGEB6P1 | RP11-420K8.1 | RP11-142A12.1 |
| GNAI1 | STRC | TBC1D26 | HAND2 | ZBED9 | RP3-512E2.2 | RP11-247L20.3 |
| ZNF771 | SCIN | NAP1L6 | TMEM155 | LINC01063 | RP11-340I6.8 | RP11-279O9.4 |
| VTN | NPY1R | IQCF5 | MAP9 | TMEM114 | RP5-837M10.4 | RP11-301L8.2 |
| CFI | TH | DSCR4 | EDIL3 | FAM215B | RP11-3L21.2 | CTD-2323K18.1 |
| FILIP1 | DCANP1 | CHRM2 | RANBP3L | OR5K1 | RP11-423C15.3 | RP11-537A6.9 |
| CDK1 | PSG11 | DDN | RNF180 | KIZ-AS1 | RP11-44N12.2 | RP11-440L14.1 |
| KNG1 | SFTA3 | IGLV4-3 | SCGB3A2 | NALCN-AS1 | RP4-644L1.2 | RP11-216N14.9 |
| PYCRL | ACTL8 | CCDC87 | SPINK1 | PRH1 | RP11-524G24.2 | RP11-406O23.2 |
| APOA1 | PCDH18 | KCNIP1 | CDC20B | IPO9-AS1 | RP11-284F21.7 | CTD-2026K11.3 |
| SPRY4 | SCN4A | MALRD1 | CARTPT | RGPD4-AS1 | RP11-10N16.2 | RP11-180C1.1 |
| MAPK10 | SELE | GRID1 | FAM170A | SETSIP | RP4-613B23.1 | RP11-140H17.1 |
| GFAP | ZMAT4 | PNMAL2 | LINC01600 | FOXD3-AS1 | RP11-407B7.1 | RP11-66B24.2 |
| CCDC103 | NKX6-3 | TP53TG1 | ADGRF2 | PLSCR5 | RP11-488P3.1 | CTB-51J22.1 |
| HSD17B6 | MAMDC2 | MRGPRG | TLX3 | SBK3 | RP11-517P14.2 | RNF103-CHMP3 |
| THEM5 | SSC5D | KCNA4 | FAM26D | LINC00689 | RP11-552D8.1 | CTD-3076O17.1 |
| EXOC3L1 | FBLL1 | GABRG3 | T | LINC01353 | RP11-542K23.7 | RP4-718D20.3 |
| FGF1 | FBXO17 | OR6B2 | IL31RA | PCAT7 | RP11-219C24.10 | SLCO4A1-AS1 |
| MALL | WBSCR27 | OR14J1 | SP8 | UTAT33 | RP11-186N15.3 | RP5-902P8.12 |
| IGFBP6 | GRAPL | MAS1L | IQUB | OR5K2 | RP5-1091N2.9 | RP5-1021I20.5 |
| FTCDNL1 | OR4D6 | OR52A1 | SHH | HOXB-AS3 | RP5-864K19.4 | RP11-800A3.4 |
| SULT1C4 | RNF222 | C5orf60 | SBSPON | KBTBD13 | RP11-173B14.4 | RP11-687M24.8 |
| CLEC2L | RGS21 | KLHL41 | EN2 | CLDN34 | RP5-994D16.9 | LA16C-4G1.3 |
| USHBP1 | GJB7 | SCARF2 | KCNV1 | SP2-AS1 | CCDC183-AS1 | CTC-260F20.3 |
| PIPOX | DACT2 | OR4A15 | CSMD3 | C11orf94 | RP11-66N11.7 | RP1-122P22.2 |
| PRSS27 | RARRES1 | WFDC10B | PLA2G2F | LINC01135 | RP13-39P12.3 | CTD-2270P14.1 |
| PKLR | SPP1 | NEUROG1 | HTR6 | IFNA17 | RP11-354K1.2 | RP13-131K19.2 |
| MAGI2 | ABCC8 | IL31 | ILDR1 | LINC01239 | RP11-404F10.2 | RP11-25H12.1 |
| ZNF71 | PCDHA12 | CTXN3 | TMC2 | MIR155HG | AF127577.10 | RP11-24F11.2 |
| PDE3A | TNNT2 | OR11H12 | PLPPR1 | Z82214.2 | RP11-20J15.3 | RP11-24J19.1 |
| MRO | SLC8A2 | HS3ST4 | OR13C4 | MLIP-AS1 | RP11-348F1.3 | B4GALT1-AS1 |
| PARD3B | PRNT | ERICH4 | CRB2 | KDM5C-IT1 | C9orf135-AS1 | RP11-523O18.5 |
| CLDN11 | ZNF541 | OR5P3 | LCN9 | TMEM229A | ATP13A4-AS1 | RP11-688G15.3 |
| FRMD6 | SLC35G3 | NXPH3 | CACNA1B | LINC01007 | SLX1A-SULT1A3 | RP5-1087E8.3 |
| NRARP | CLDN4 | SPIRE2 | ANKRD1 | RNF224 | RP11-127B16.1 | AC005609.16 |
| GPRC5C | LCTL | LY6G6D | CYP17A1 | LINC00167 | RP11-474D1.3 | RP11-380L11.4 |
| DHRS2 | MSX2 | TMEM240 | INA | LINC01346 | RP11-717D12.1 | RP11-297D21.4 |
| LAMB2 | CYP4F8 | FAM87A | ZNF214 | IFNA13 | RP11-1036E20.9 | RP11-374N7.2 |
| NR5A2 | DEPDC4 | TBPL2 | GLYAT | TTTY15 | RP11-429E11.2 | AL050303.10 |
| GNA14 | SPEM1 | LHFPL1 | OR5F1 | C16orf82 | RP11-445O16.3 | RP11-227G15.12 |
| ZNF81 | PDE1A | PCDHA5 | HTR3B | ETV5-AS1 | XX-FW81066F1.2 | RP11-245P10.8 |
| HOXD9 | WDR97 | RXFP3 | GLB1L2 | SULT1A4 | RP5-1142A6.2 | RP11-675P14.2 |
| MLPH | PPP5D1 | FIRRE | GGTLC1 | KRTAP4-12 | LL0XNC01-16G2.1 | IGF2BP2-AS1 |
| ABCA3 | SYNPO2L | OR6J1 | ZP1 | MRGPRX3 | RP11-295P9.6 | CTB-60E11.9 |
| HPD | ILIA | GPC2 | KCNE4 | CYP4X1 | RP4-758J18.10 | CTD-2583A14.8 |
| AQP7 | INTU | KCNJ14 | CDH22 | SLITRK2 | RP11-57H14.3 | RP11-816J6.3 |
| KRT18 | MOGAT2 | FAM221B | TRIM48 | LEMD1 | RP3-333H23.8 | RP11-138I18.1 |
| MYO5C | CSPG5 | VIT | OR8K1 | ATP4B | AC009892.10 | CTB-147C22.6 |
| LAMA5 | COL14A1 | PSG5 | MIA2 | TAL2 | RP11-222A5.1 | RP11-442O1.3 |
| TBX1 | TMEM130 | LRRC10B | LYPD1 | ZPBP2 | RP11-374M1.4 | RP11-213H15.4 |
| FGA | A2ML1 | GALR2 | GPM6A | LRRC70 | RABGAP1L-IT1 | CTC-448F2.7 |
| HTRA1 | PALM3 | BRICD5 | SLC2A13 | OR5D18 | RP11-223P11.3 | RP11-416H1.2 |
| DES | ASCL4 | BLACE | MSGN1 | TAS2R42 | CTD-2296D1.5 | AC015849.16 |
| SEMA3D | WNT5A | NTM | ADAMTS12 | FFAR4 | RP11-359E8.3 | CTC-523E23.11 |
| GPNMB | HELT | OR10G7 | ANO4 | SLC51B | RP5-1172N10.2 | RP11-435O5.5 |
| SIX5 | SLC7A2 | PCDHA13 | PABPC3 | SOWAHB | RP11-33A14.1 | RP4-591C20.9 |
| SPC24 | CLDN19 | LRIT3 | GFRA1 | LCE1E | RP5-1033H22.2 | RP5-1181K21.4 |
| CDH13 | ENPP6 | OTOL1 | TMEM132D | GPAT2 | RP11-433C9.2 | RP11-97O12.5 |
| PLCH1 | HHATL | IQCJ | IFNK | SLC36A2 | RP3-522D1.1 | RP11-256I23.2 |
| RARRES2 | GPR176 | MCCD1 | IFNA16 | DEFB132 | RP11-371A19.2 | RP11-173C1.1 |
| MAPK11 | PRSS22 | OR8H1 | CER1 | KRTAP19-4 | RP11-179K3.2 | CTB-139P11.2 |
| SNCAIP | PLA2G2A | OR5T2 | FAM83A | AKAP14 | RP11-402G3.3 | RP13-516M14.10 |
| ADRA2A | CLDN3 | AGTR2 | SLC10A6 | C5orf38 | RP11-469A15.2 | CH507-42P11.8 |
| DMD | CAMK1G | URAD | GLRA3 | OR9Q1 | RP11-69I8.2 | RP11-497G19.7 |
| RHPN2 | FAT3 | RP1L1 | CDH18 | CEACAM19 | RP5-1063M23.2 | RP11-542M13.3 |
| CHRNA2 | PRR19 | OR4A47 | MEGF10 | LRRC16B | RP11-1008C21.2 | RP1-102K2.8 |
| PRDM16 | PCDHAC2 | MEG3 | LECT2 | MAGEE2 | RP11-544A12.4 | CTD-2192J16.22 |
| EDNRB | PKNOX2 | CECR7 | MYLK4 | CYP27C1 | RP11-365O16.6 | CTD-3032J10.3 |
| TMEM238 | GREB1L | UPK3B | PSD2 | CFAP73 | RP1-142L7.5 | CTD-3154N5.2 |
| CXorf36 | OR7A5 | SHISA9 | SLC17A4 | MPPED1 | RP11-475I24.3 | CH507-216K13.2 |
| FAM13C | IL17REL | OR52E6 | HIST1H2BA | KRTAP17-1 | RP11-509J21.2 | RP11-629B11.5 |
| CD276 | SMTNL2 | CFAP57 | HMGCLL1 | OR13F1 | RP11-473M10.3 | RP11-1334A24.5 |
| MT1E | CAMKV | FDCSP | TTBK1 | FGF3 | RP11-147C23.1 | RBAK-RBAKDN |
| GTF2A1L | TMEM52B | OR2Z1 | TCTE1 | SERPINA11 | RP5-947P14.1 | KB-1572G7.2 |
| CDKN2A | NKPD1 | TUBA4B | CYP39A1 | P2RY4 | RP11-206L10.9 | RP11-131M11.3 |
| ANTXR1 | PRRX1 | BCAR4 | FAXC | TNMD | RP11-356I2.4 | RP11-474G23.3 |
| RAB3C | RNASE7 | GPR19 | GPR6 | LCE3E | AC114730.11 | RP11-216L13.19 |
| SEC14L5 | TMEM136 | HCG9 | SLC2A12 | LCE3A | RP11-204P2.3 | RP11-165A20.3 |
| TBX3 | CYP8B1 | PRORY | PNLDC1 | FAM186A | RP11-334A14.8 | RP11-7F17.8 |
| SLC2A5 | SPRR2A | OR4C6 | VIP | ODF4 | XXbac-B33L19.4 | RP1-170O19.23 |
| GINS4 | TMEM215 | ZFP57 | IGFBP1 | KRTAP6-1 | ARHGEF7-AS2 | RP11-798M19.6 |
| SLC35D3 | POU6F2 | FAM43B | SSC4D | FAM110C | RP3-470B24.5 | RP1-170O19.22 |
| SOX5 | PLCXD3 | GPC6 | TMEM47 | DDX53 | RP5-1011O1.3 | RP11-517I3.2 |
| NPNT | MYO15A | ASPDH | SLC16A2 | DRD1 | RP11-148B18.4 | RP11-405M12.4 |
| L3MBTL4 | SPACA4 | WBP2NL | CPXCR1 | CLCNKB | RP11-316M21.7 | RP11-820I16.4 |
| TEK | OR2V2 | SLCO6A1 | FRMPD3 | FAM227A | RP11-525G13.2 | RP11-58C22.2 |
| IFI27L1 | ZNF853 | PCP4 | FATE1 | SLC22A10 | RP5-865N13.1 | RP11-345K9.2 |
| HJURP | TSPAN10 | OTOP2 | RGS20 | ROBO2 | RP11-320G24.1 | RP11-83A24.2 |
| IL13RA2 | ACR | PTCHD4 | TRIM55 | NT5C1B | RP11-10O22.1 | ABHD14A-ACY1 |
| NKX2-3 | FZD8 | OR6S1 | ATP6V0D2 | SGCZ | RP11-379B18.5 | RP11-503P10.1 |
| APOD | OR1I1 | MUC21 | RSPO2 | EFCAB10 | RP4-781K5.5 | RP4-734G22.3 |
| C1S | GXYLT2 | PRCD | MCHR2 | KRT76 | PRICKLE2-AS1 | RP11-637O19.3 |
| RGS5 | OR1D2 | OR52E8 | POU4F1 | MIXL1 | RP11-288I21.1 | RP11-147L13.15 |
| ZDHHC1 | OR4Q3 | TMEM213 | PFKFB1 | PPIL6 | RP11-690C23.2 | RP11-156E8.1 |
| COL3A1 | FAM132A | PRR36 | ITGAD | KPNA7 | RP1-90G24.10 | RP11-222K16.2 |
| BATF3 | KCNB2 | MT1A | CYLC2 | SV2B | PRICKLE2-AS1 | RP5-994D16.11 |
| PCDH17 | RNASE8 | TRBV7-6 | TRIM42 | FAM131C | C1QTNF9-AS1 | CTD-2619J13.5 |
| GATA4 | SIRT4 | ADGRB1 | MICU3 | TLCD2 | AC093627.10 | RP11-443B20.1 |
| FCN2 | LHX5 | DPRX | KIF5A | DBX2 | RP11-656D10.6 | RP11-424N24.2 |
| GATA6 | DSCAML1 | RGAG1 | CFAP70 | KRT79 | RP11-31F15.2 | RP11-302M6.4 |
| MEOX1 | HAP1 | KPRP | WIFI | C11orf87 | RP5-1121E10.2 | RP3-337H4.10 |
| APOC2 | BCAS1 | RTP5 | MMP16 | CLDN24 | LL0XNC01-237H1.2 | RP11-136L23.2 |
| SPA17 | REP15 | OR2AG2 | ADAMTSL3 | NLRP9 | RP11-90K6.1 | RP1-313I6.12 |
| AR | IQGAP3 | OR10D3 | ART3 | NPAP1 | RP11-477J21.6 | bP-218909.3 |
| GLIS3 | TLL2 | LDLRAD2 | CLDN17 | TMEM210 | RP11-16N2.1 | RP11-855A2.1 |
| FBXL7 | TLX1NB | OR51I2 | CLDN8 | KLHL34 | RP11-499P20.2 | RP5-1174N9.2 |
| ALPK3 | TPSD1 | LRRC74B | KCNS2 | NKAIN3 | RP11-185E8.1 | RP11-803B1.8 |
| PTK7 | NIPAL4 | TMEM72 | PRG3 | OR13C8 | RP11-429G19.3 | LLNLR-284B4.1 |
| S100B | OR51M1 | PRB4 | COX6A2 | KRTAP15-1 | RP5-955M13.4 | CTD-2349P21.7 |
| TOM1L1 | RALYL | ANKRD63 | MPV17L | ZNF177 | RP11-29B9.2 | RP11-797D24.4 |
| OVOS2 | PRSS55 | DSCR9 | GRID2 | NUTM2G | CTA-125H2.2 | CTA-228A9.3 |
| ZSCAN31 | SPAG4 | OR6N2 | SST | KRTAP10-6 | RP4-706G24.1 | RP1-225E12.3 |
| ADCY6 | ERICH5 | ENO4 | HTR5A | OTOG | AC011899.10 | RP11-182L21.6 |
| MAPK12 | C2orf54 | IQCF3 | CLEC18A | LINC00265 | RP11-53B5.1 | RP11-314N13.10 |
| CDH11 | PATE3 | ADGRA1 | ARMC12 | POTEE | RP11-87N24.3 | RP5-1024N4.4 |
| PLCD4 | GGT5 | PCDHGB1 | ARSE | RUFY4 | RP11-240L7.4 | RP11-152N13.16 |
| SDC1 | SUSD2 | FAM166A | C9orf43 | OR6C6 | AC008440.10 | RP11-219D15.3 |
| BFSP1 | EPHB3 | COL4A5 | EXTL1 | BSPH1 | RP11-234K24.3 | CTD-2574D22.6 |
| MT1G | CEP170B | LRP1-AS | NLRP14 | H3F3C | RP11-132A1.4 | RP11-258F1.1 |
| GJA1 | MISP | TRBV5-4 | PTPDC1 | IFNA2 | OSBPL10-AS1 | CTD-2535L24.2 |
| BMP4 | PURG | C2CD4D | ESYT3 | JAKMIP3 | KIAA1614-AS1 | AP000275.65 |
| PTPRR | KCND2 | RTL1 | FAM46B | INSC | RP11-388P9.2 | RP13-516M14.4 |
| FAM81B | OR4N5 | CDK3 | RHBDL2 | FAM72B | RP11-536K7.3 | CTA-299D3.8 |
| FAM110D | H2AFY2 | DMBX1 | NRG2 | RNASE9 | RP11-98D18.2 | RP11-849F2.5 |
| ANKRD29 | COLEC10 | LHFPL3 | CD1B | KRTAP13-4 | RP11-334A14.5 | RP11-817J15.3 |
| SDCBP2 | CES3 | CCER1 | FZD7 | DUPD1 | RP5-930J4.2 | RP11-387H17.6 |
| ABI3BP | PKP3 | AMTN | ALS2CR11 | OSTN | RP5-1120P11.3 | CTD-2532D12.5 |
| AZGP1 | KIRREL | CIDEC | OTOA | FSIP2 | CTD-2330K9.2 | CTB-58E17.5 |
| NR0B2 | FOLH1 | NPSR1 | LRGUK | FZD9 | RP6-109B7.2 | CTB-25B13.9 |
| TCF15 | TMEM119 | LHFPL5 | MEPE | SKOR1 | RP1-140A9.1 | RP11-218M11.6 |
| RSPH1 | UBQLNL | HOXC6 | CAPSL | PLA2G2E | RP1-207H1.3 | C20orf166-AS1 |
| NR2F6 | UBQLN3 | OR52M1 | DDX4 | CNR2 | RP11-343N15.2 | RP4-777O23.3 |
| GLIS2 | SUGCT | WAC-AS1 | ACMSD | GLRA4 | RP11-374M1.2 | RP13-347D8.7 |
| MMP28 | MAMSTR | SAMD11 | SYCP2L | ENTPD8 | RP4-669H2.1 | CTC-490E21.14 |
| MFAP5 | PAX2 | PLEKHN1 | CCDC148 | FAM78B | RP11-382E9.1 | RP11-285E9.6 |
| CIART | LSINCT5 | NHLRC1 | PLA2R1 | ASTL | AC073342.12 | RP11-498C9.15 |
| DNAH10 | PRIMA1 | PCDHB11 | FEZF2 | C9orf152 | RP11-327P2.5 | CTD-2527I21.15 |
| CHAC1 | PHKA1 | OMP | FAM92B | AADACL3 | RP11-5P18.5 | RP11-443F4.1 |
| ISLR | OR51G2 | WASIR2 | SPHKAP | LRRC66 | CCDC144NL-AS1 | RP11-834C11.15 |
| CCNB2 | GLI2 | PRR27 | ANKFN1 | FAM25A | RP11-1275H24.1 | RP11-961A15.1 |
| PTGIS | C2CD4C | OR6Y1 | SLC5A10 | MESP2 | RP11-465N4.4 | CTD-2194A8.2 |
| ATXN8OS | OR51G1 | SSPO | CHST9 | PLA2G4E | RP1-293L6.1 | RP1-59D14.5 |
| GULP1 | OR10H4 | PRPS1L1 | CDH12 | RNF133 | XX-C2158C12.1 | RP11-542C16.1 |
| ADGRL2 | CALHM3 | ANHX | GAL3ST2 | C17orf82 | RP11-109I13.2 | RP1-179P9.3 |
| GC | NKX2-5 | RASSF10 | ABCA10 | KRTAP21-2 | RP11-80H5.9 | RP11-143K11.7 |
| CAPN11 | TEX35 | SH2D5 | PPP1R3A | TMPRSS6 | RP1-65J11.5 | RP11-835E18.5 |
| SLC22A7 | OR4C5 | OTUD6A | GPR26 | TMPRSS11A | RP11-666F17.1 | C1QTNF1-AS1 |
| MYOZ3 | OR4C3 | OR2M3 | CCDC173 | OR2AK2 | RP11-25K21.1 | RP11-788M5.4 |
| SPDYC | OR1J4 | LCE1C | MMP21 | CCK | RP11-421L21.3 | ABC7-42404400C24.1 |
| MOG | FOXL1 | ORM2 | PSMA8 | VSTM2B | RP11-12C17.2 | CTD-2201E18.3 |
| EGFR | OR52E2 | SPANXN4 | CXADR | FOXD3 | RP11-760D2.5 | CTB-102L5.4 |
| CDC25A | PFN4 | ESRRG | ADAMTS5 | SPATA21 | RP11-318G21.4 | CTC-277H1.6 |
| LIPH | GPR152 | FAM66C | CA10 | LCE4A | LA16C-23H5.4 | RP11-63A1.4 |
| IQSEC3 | OR52W1 | SPTSSB | RSPH10B | LCE2A | RP5-875O13.1 | CTD-3193K9.3 |
| SMAD9 | GCKR | PFN3 | CNTNAP5 | LCE2C | RP11-547D24.1 | RP11-266K4.13 |
| NXF3 | TSKU | OR5AC2 | ODF1 | DCC | RNASEH2B-AS1 | CTD-2659N19.2 |
| HRCT1 | GALNTL6 | TEX41 | MAGEC1 | HYPM | SLC26A4-AS1 | RP11-666A8.7 |
| LAYN | TRPM3 | KIF4B | GRIA1 | POTEG | RP11-567G24.3 | CTC-459F4.3 |
| MAT1A | GLP2R | TUG1 | LDHAL6A | OR5W2 | RP4-799D16.1 | CTC-510F12.6 |
| OBSL1 | KLK15 | IFNA14 | ELFN2 | HAPLN4 | RP11-168K11.3 | RP11-727F15.11 |
| SYBU | SERTAD4 | SFTA2 | MIR202HG | ERAS | CTC-242N15.1 | CTD-2619J13.8 |
| CRCT1 | GPR1 | OR2T2 | CST6 | SLC18A3 | RP11-61L14.6 | CTC-435M10.6 |
| PARD6G | PCDHB4 | BECN2 | DIRC1 | FAM69C | ARHGEF19-AS1 | RP11-455P21.3 |
| AGXT | OR9G1 | CGB7 | C11orf45 | C2orf78 | B3GALT5-AS1 | RP11-316O14.1 |
| FAM109B | CDH7 | OR10J1 | TRHR | OR6C75 | RP13-786C16.1 | CTB-33G10.11 |
| SMKR1 | CD164L2 | OR6K2 | ZCCHC12 | DNER | IGHV1OR15-9 | CTD-2291D10.4 |
| LHX6 | ROPN1 | ELAVL3 | IGDCC3 | CPSF4L | RP11-723O4.6 | CTD-2619J13.9 |
| LHFP | RIPK4 | POLR2J2 | SLC26A9 | ZCCHC13 | RP11-381O7.3 | RP5-1057I20.4 |
| ITIH1 | RDH8 | ZNF676 | GOLT1A | C1orf204 | RP4-539M6.14 | RP11-457D20.2 |
| TPD52L1 | HOXB4 | NKAPL | PABPC5 | C19orf67 | RP5-821D11.7 | RP11-845C23.3 |
| FOXC1 | PKD2L2 | OR56A5 | GPR149 | NRN1L | RP11-294C11.1 | RP11-866E20.3 |
| MID1 | FGF20 | OR14A16 | OR5J2 | FAM19A3 | RP11-863K10.7 | RP11-879F14.3 |
| MAATS1 | OR51F2 | OR52K1 | WFDC5 | SLITRK6 | CTB-158D10.3 | RP11-1094M14.11 |
| KRT13 | INHBC | SPRR2B | FAM182B | MAGEA12 | RP11-46C24.3 | CTD-2008L17.1 |
| SMARCA1 | CHST1 | GOLGA8M | ANKS4B | MGAT4C | RP11-830F9.6 | AC002398.12 |
| CSRP2 | SCUBE2 | NANOS1 | ISX | SLX1B | CTC-425O23.2 | CTD-2293H3.2 |
| PRSS36 | OR10P1 | NKAIN2 | ACBD7 | ANKRD62 | DKFZp779M0652 | CTB-129P6.4 |
| CADPS2 | RIMBP2 | CHRNG | CHRNA7 | TNFSF15 | RP11-401P9.6 | RP11-42O4.2 |
| CHADL | ADRB1 | ELFN1 | GPR156 | OR8K5 | RP11-111F5.4 | RP11-80P20.3 |
| TCEAL2 | OR2D3 | OR11G2 | LINC00521 | OR8H3 | RP11-126H7.4 | RP11-713P17.4 |
| MCAM | FFAR3 | TMEM26 | C11orf44 | TMEM252 | RP4-640H8.2 | RP11-90D11.1 |
| GJD3 | CKM | OR2L8 | CALCB | ODF3L2 | RP11-219G17.4 | CH507-145C22.1 |
| STXBP6 | KRT86 | OR2G6 | CREG2 | OR5AS1 | RP11-65D24.2 | RP11-10N23.2 |
| UTF1 | HTR1A | NTF4 | WBSCR28 | FTMT | TRAV38-2DV8 | RP11-736N17.10 |
| PYCR1 | OR5A1 | COL25A1 | TSPEAR | OR4P4 | RP3-416J7.5 | CTD-2562J17.7 |
| FUT1 | DNAJC22 | S100A2 | PLEKHD1 | OR4A16 | RP1-122K4.3 | CTD-2647L4.1 |
| C3orf70 | VSTM2A | PCDHGB4 | TMPRSS7 | PNMAL1 | RP11-563J2.3 | RP11-246E12.2 |
| RIMS2 | FSD1 | FAM111B | ENTHD1 | USH1G | ZNF625-ZNF20 | RP11-10A14.3 |
| CLEC14A | CEACAM5 | RELN | OR11H4 | TRIM49B | RP11-798L4.1 | RP11-865I6.2 |
| SMIM10 | OR5D14 | HRH1 | OR11H6 | OR4F15 | RP11-363N22.3 | RP11-333A23.4 |
| CPLX1 | TEX38 | KIR2DL4 | SNX31 | ENPP7 | CTD-2008L17.2 | RP11-697M17.2 |
| PLEKHH3 | SRL | FAM196A | ARL13A | IZUMO1 | CTD-2308N23.2 | RP11-383J24.1 |
| SYNPO2 | RBMXL2 | GJB3 | GSTA3 | DCAF4L1 | RP11-465L10.10 | RP11-386G21.1 |
| MAP2 | NRG3 | HMX2 | SPERT | MBD3L3 | CH17-340M24.3 | RP11-632L2.2 |
| HSPB7 | RHOV | TMCO2 | FAM71D | KIAA2012 | RP11-98D18.9 | CTD-3239E11.2 |
| SPATA24 | PDIA2 | PRELP | OR4D9 | C17orf77 | RP11-375H19.2 | RP11-567J20.2 |
| NOS1 | HHIPL1 | TEX22 | COL6A5 | KBTBD3 | RP11-163F15.1 | RP11-555G19.2 |
| RHBDF1 | CCDC188 | TMEM198 | OR5B3 | CDY2A | RP11-335E6.2 | RP11-211C9.1 |
| DSP | FIGN | TMEM253 | OR10W1 | CAV3 | RP11-84A19.2 | CTB-131B5.5 |
| RN7SL1 | SPRR2F | SLIT1 | KRT2 | EPGN | RP11-332M2.1 | RP11-51J9.4 |
| HSPB6 | LSAMP | NOTUM | MRGPRF | KRTAP11-1 | RP11-296O14.3 | CTC-756D1.3 |
| OXTR | NCBP2L | C7orf65 | SLC22A13 | RESP18 | RP11-566K11.2 | CTC-209H22.3 |
| MET | PGK2 | OR2V1 | XKR3 | PAPPA | RP11-544M22.1 | RP11-67H2.1 |
| MMRN2 | PRSS54 | TMEM105 | FRG2C | MAFA | RP11-208N14.4 | RP11-10C8.2 |
| MYRIP | UBL4B | SCN10A | ADAMTS20 | GPR135 | AP001189.4 | RP11-108K14.12 |
| GLYATL1 | BARX2 | SPPL2C | MAB21L3 | OR8D4 | AC132217.4 | RP11-809O17.1 |
| MTCL1 | PAX4 | WBSCR17 | C11orf86 | OR5M8 | AC069368.3 | RP11-281O15.4 |
| CCL19 | MYT1L | OR4A5 | GPR151 | OR5AK2 | AC093818.1 | KB-1410C5.2 |
| LRRC20 | KCNK9 | RTN4RL1 | CST9 | C1orf194 | AC090498.1 | CTD-2033D15.3 |
| LEFTY1 | PTPRZ1 | MAGEA11 | IQCF1 | ZNF648 | PITPNA-AS1 | RP11-806O11.1 |
| CCL2 | COBL | BRINP2 | SAA1 | C3orf80 | AC006538.4 | KB-1107E3.1 |
| SCNN1B | PSAPL1 | PAX9 | TMEM196 | NKX2-6 | AC006449.2 | RP11-489E7.1 |
| SLC2A4 | GJB1 | MUC2 | RNASE11 | ACTA2-AS1 | ZNF561-AS1 | CTD-2517M22.16 |
| VWA1 | BNC1 | CCDC190 | CSPG4 | FAM71C | ZNF667-AS1 | RP11-1007G5.2 |
| HR | OR6P1 | OR6C76 | C2orf70 | RRH | AC093323.3 | LA16C-390H2.1 |
| IGF1 | PITX1 | OR2A14 | XCR1 | ADGRD2 | ZFP91-CNTF | CH507-338C24.1 |
| UNC13A | ZNRF4 | PRAME | APOBEC4 | PLD5 | AC012507.4 | RP11-789C7.1 |
| PRR16 | CFAP69 | SHC4 | HES3 | WFDC10A | AC022182.3 | RP11-9N12.2 |
| SLC5A1 | GSX1 | EPS8L3 | OR1L1 | ALX1 | AC005614.5 | RP5-1142A6.10 |
| MCAT | GFY | UBTFL1 | SUSD5 | CCDC129 | AC008074.1 | RP11-150O12.3 |
| BAMBI | PDE4C | OR10G8 | C1orf100 | DEFB123 | AC079742.4 | CTD-2292P10.4 |
| CCR10 | KDM4D | OR3A2 | PIFO | PRR26 | AC005339.2 | RP11-131M11.2 |
| DLL4 | SLC5A5 | TMEM257 | LRRC63 | ZSCAN4 | AC012146.7 | RP11-115C10.1 |
| NMNAT2 | LILRB5 | OR6B1 | C10orf111 | EIF2S3L | PARD6G-AS1 | RP11-350F16.1 |
| HSD3B7 | HMP19 | C1orf95 | OR4L1 | SLC47A2 | TRIM52-AS1 | RP11-11N7.5 |
| CABP1 | BEND3 | TCP10 | C15orf48 | C3orf22 | AC093627.8 | GS1-251I9.3 |
| ARNT2 | RNF150 | RPTN | C9orf106 | CLRN3 | AC015688.3 | RP11-158I9.8 |
| LRRC26 | OR4D5 | FANK1 | HTR3C | HOXC9 | ST6GALNAC1 | CTB-58E17.1 |
| CENPM | MEDAG | LELP1 | GOLGA8Q | MAP3K15 | AC078883.3 | RP11-39H3.2 |
| WWC1 | OR7A17 | TUSC5 | FAM27L | MAP6D1 | ZNF529-AS1 | SENP3-EIF4A1 |
| ANKS1B | ZNF556 | C1orf68 | SPINK6 | GREM2 | AC016700.5 | RP11-399B17.1 |
| FLNC | WDR87 | MROH2A | VN1R1 | GPR137C | AC007246.3 | CTD-2574D22.7 |
| WFDC1 | WT1 | FAM19A5 | C9orf62 | ADIPOQ | AC103564.7 | XXbac-B562F10.12 |
| ACSBG1 | HRH3 | SORCS2 | NEUROG2 | MUC16 | AC145212.4 | RP5-965G21.3 |
| BFSP2 | BMP7 | MFRP | LINC00843 | TMEM132C | AC003075.4 | RP11-664I21.5 |
| GTSF1 | LCN12 | OR4C46 | ERBB4 | SLC25A41 | AC068533.7 | RP11-17A4.2 |
| SH2D4A | SLC35G5 | ASCL5 | OTOS | KRTAP13-2 | AC108488.4 | RP11-626G11.6 |
| SYDE1 | KLF17 | VSIG10L | CSRNP3 | TMEM95 | AC005779.2 | RP11-779O18.1 |
| SLC12A5 | ADRA1D | CERS1 | DYNAP | C6orf58 | AC011558.5 | AC002310.14 |
| AGBL4 | SALL4 | AADACL2 | GDPD4 | OR2T10 | AC007906.1 | RP13-20L14.2 |
| RFTN2 | OR52A5 | QRFPR | TAS1R2 | BMP8A | AC005076.5 | RP11-32K4.1 |
| CCDC114 | SGK2 | DLGAP2 | HTR1F | MACC1 | AP003774.4 | OSGEPL1-AS1 |
| ONECUT1 | CAMK2B | KRT14 | OR4K13 | ACSM2A | AC005253.4 | RP11-219B4.3 |
| PLLP | KCNK4 | FOXE3 | MTUS2-AS1 | PAPL | AC003002.4 | RP11-398G24.2 |
| SOX7 | REG3A | CALHM1 | LINC00311 | KCTD16 | ZNF790-AS1 | RP11-10N23.4 |
| ADH1C | GAP43 | ZNF735 | SMCO3 | B3GALT5 | AC005786.7 | RP11-539E17.5 |
| NPM2 | EFS | PRG2 | ZCCHC5 | KCTD8 | ZNF571-AS1 | RP11-755F10.3 |
| CBR3 | SRY | OR2L3 | TMEM31 | EMILIN3 | GPR75-ASB3 | RP11-1123I8.1 |
| ZNF584 | RPRM | FAM47C | C1orf101 | PNMA3 | AC005037.3 | XXbac-B33L19.12 |
| HOXA4 | OR1A1 | HAGLR | FJX1 | GPR39 | AC037459.4 | RP13-895J2.4 |
| DRC7 | CCL11 | CCDC79 | MKRN3 | CNGA2 | AC007249.3 | RP11-212I21.3 |
| CRTAC1 | LRRC74A | MEIG1 | ALOX12B | SCN5A | SDCBP2-AS1 | CTD-2340D6.1 |
| FLRT2 | SYCE1 | OR10G4 | LSMEM2 | LRRC55 | AC010226.4 | RP11-114F3.4 |
| ADSSL1 | MROH8 | CYP4A11 | OR6C4 | NPW | ZNF436-AS1 | RP11-49K24.9 |
| MASP1 | PNMA2 | KANTR | RPRML | RGS7 | AC007283.5 | RP1-149L1.1 |
| TMEM37 | MLNR | HES5 | ARL14 | TRIML1 | AC114273.3 | RP11-702H23.4 |
| SH3RF2 | TEX33 | ACTL7A | PRSS42 | SPRR4 | ZNF503-AS2 | RP11-697K23.3 |
| PHLDA3 | UGT2B7 | DSCR10 | C2orf73 | GPR173 | AC109486.1 | RP11-12J10.4 |
| SLIT3 | FAM26E | SULT1C2 | CAPZA3 | GOLGA6L4 | AC114752.3 | RP1-170O19.17 |
| LMO3 | LURAP1 | OR9Q2 | YIPF7 | SIX6 | AC018816.3 | RP11-108E14.1 |
| PLA1A | SCML2 | PRR31 | HMGB4 | MRGPRE | AC009758.1 | RP11-254I22.3 |
| GSTA1 | ZNF479 | KLHL32 | OR4F21 | ACTRT3 | AP000487.5 | CH17-140K24.5 |
| BOC | LAMA3 | CNGA1 | OR4K5 | A3GALT2 | AC046143.3 | RP11-1090M7.3 |
| ANK2 | KSR2 | LCE5A | OR4N2 | NCMAP | AC010084.1 | RP3-333H23.9 |
| CCNA2 | CHRDL2 | SCX | DCAF4L2 | BPIFC | AC004813.1 | CTD-2574D22.2 |
| KRT222 | ST8SIA5 | SAPCD2 | OR4X1 | OR56A3 | AC079767.4 | RP11-319G9.1 |
| GPC5 | CDH20 | PRR30 | TTTY14 | POU3F2 | AL513523.2 | RP11-491F9.2 |
| LAMC3 | PRAMEF8 | ATP5L2 | OR51V1 | NUTM1 | AC108004.3 | RP11-629N8.4 |
| PTGER3 | OR1L3 | PCDHA11 | LINC00303 | PSG9 | KRTAP10-11 | CTC-338M12.9 |
| STAP2 | OR1L8 | HTR3E | OR51L1 | FAM19A1 | ZNF337-AS1 | RP3-468K18.7 |
| LRRC75B | OR1N2 | OR2A1 | GRIN1 | KLHL25 | AC004837.4 | RP11-499E18.1 |
| USP13 | NPFFR2 | PRR21 | OR51T1 | C11orf88 | TIPARP-AS1 | CH507-154B10.1 |
| AOX1 | RXFP1 | OR4C12 | TRIM60 | TCEAL7 | AC004853.1 | CTD-2139B15.2 |
| CXCL9 | SYNDIG1 | MYCNOS | IFNW1 | SOX1 | AC069277.2 | RP11-297C4.6 |
| MYLPF | L1TD1 | HSPB9 | ZDHHC22 | ZNF662 | GLYCTK-AS1 | RP1-47M23.3 |
| CCDC40 | IFNE | CCT8L2 | CETN1 | OR1G1 | MAN1B1-AS1 | RP4-800O15.1 |
| DOK6 | CCDC184 | KLK12 | RIMKLA | FREM3 | RP11-6O2.4 | RP11-159H10.3 |
| CYP26B1 | EPPIN | PCLO | OR2M7 | NEB | AC005625.1 | RP11-7F17.5 |
| DBNDD1 | OR51E1 | FAM228A | OR2T33 | OR2T11 | AC004448.5 | CTD-2013N17.6 |
| PRSS50 | CCNI2 | SPATA12 | OR2AJ1 | GABRR3 | KCTD21-AS1 | RP11-25L19.1 |
| CDKL3 | EPO | TWIST2 | LRRN4CL | POTEC | AC073254.1 | RP11-252E2.1 |
| NPHP1 | AVPR1B | KRT26 | UBE2U | WT1-AS | AP000476.1 | LLNLR-259H9.1 |
| PSG4 | TUBA3C | ZNF442 | OR2T8 | OR51B4 | AP000253.1 | RP11-454F8.3 |
| AKR1C3 | ACE2 | PRR23C | RBM44 | PLGLB1 | AC005262.2 | RP11-479O17.9 |
| TPH2 | ATP2B2 | THBS2 | NHLH2 | CCBE1 | AC009005.2 | RP11-902B17.1 |
| LRIG3 | SLC6A1 | OR10T2 | MAGEB10 | TISP43 | AC016757.3 | RP11-44N21.3 |
| ETNK2 | ZSCAN10 | GABRA5 | MOS | FAM9A | AC026248.1 | RP11-290O12.2 |
| MLXIPL | SYN2 | IGFL1 | OR10AD1 | SPDYE4 | AP000654.4 | RP11-574K11.26 |
| NDN | HBE1 | TSPAN6 | MUCH | REC114 | RAET1E-AS1 | RP11-145P16.2 |
| PCDHGC3 | FAM95B1 | LHX2 | GP2 | C10orf107 | AP003068.9 | AE000658.31 |
| FAM187A | MLIP | PGLYRP4 | SOX14 | TMEM89 | AC114730.3 | CTC-312O10.3 |
| G6PC | KCNA5 | IVL | SFTPB | C19orf71 | AC112721.1 | RP11-768F21.1 |
| REEP2 | HYDIN | ECM2 | CTRB1 | TRIM61 | AF131216.5 | RP11-76C10.3 |
| EXO1 | PLPPR3 | BNIPL | ZBBX | GRIN2A | AC004540.4 | RP11-1079K10.3 |
| FAM167A | AIPL1 | INHBB | C8orf46 | C10orf120 | AC007679.3 | CTC-236F12.4 |
| PODNL1 | CREB3L1 | CCDC74A | ARMC4 | PGPEP1L | KCNIP2-AS1 | RP1-130H16.18 |
| MMP2 | TRIM7 | C2orf48 | GSG1L | GKN2 | AC147651.4 | RP11-45A17.3 |
| ANO1 | TUBB3 | CA9 | MN1 | DGCR6 | TMEM26-AS1 | CTB-129O4.1 |
| PHACTR3 | ZFP37 | SPAG8 | OR10G3 | HUS1B | AC013472.3 | RP11-196B3.2 |
| GPR45 | OR11A1 | NEUROD1 | RAB3B | VHLL | AC015922.6 | RP11-184M15.2 |
| GALNT15 | FBXO43 | KCNJ3 | GPRIN1 | DUSP21 | RP1-4G17.5 | RP11-69J7.1 |
| POLN | ZNF90 | KCNF1 | KCNAB1 | CLLU1OS | AC005062.2 | RP11-344G13.1 |
| HSD17B3 | SLC7A10 | PM20D1 | STK32A | KRT40 | MCM3AP-AS1 | CTD-2306M5.1 |
| BHMT2 | FMN2 | FOXF2 | PDILT | GRXCR2 | MRGPRG-AS1 | RP11-247C2.2 |
| RIMS1 | SAXO1 | LGI1 | RSPH10B2 | PCDHA9 | AP001615.9 | RP11-10A14.5 |
| DCLK1 | ADAMTS7 | VANGL2 | OR5T3 | PCDHA8 | AP003774.1 | RP11-386B13.4 |
| GALNT9 | TMEM239 | NXPE4 | SPRR1A | PCDHA4 | RASSF8-AS1 | RP11-326A13.2 |
| KRT7 | PEG3 | DLX4 | CCDC8 | PCDHA2 | AC083864.4 | CTC-463A16.1 |
| ZNF135 | SHROOM2 | TTLL10 | ZNF280A | PCDHA1 | AC021218.2 | RP11-452C8.1 |
| FAM124A | ASB10 | WNT4 | GKN1 | PABPN1L | AP001469.9 | CH17-140K24.2 |
| LRRTM4 | PNCK | HOXC13 | DRD5 | OR5D16 | TTC39A-AS1 | RP11-553P9.2 |
| MYCN | GPR179 | GABRB1 | CHRNA5 | OR5L2 | AC007009.1 | CTD-2095E4.4 |
| ME3 | GATA5 | ALPI | ACTRT2 | C2orf91 | ERICH6-AS1 | CH507-154B10.2 |
| ZNF77 | MNX1 | KIF12 | TACR3 | LINC00898 | AP000251.3 | RP11-83C7.3 |
| GRAMD2 | CXCL13 | BSN | CALML6 | ARID3C | AC074286.1 | RP11-509E10.3 |
| ACSM5 | MB | MFI2 | TM4SF4 | C7orf66 | AC004237.1 | RP11-479O16.1 |
| ABO | FUT6 | HEYL | OTUD7A | LINC00654 | AC004538.3 | CH507-210P18.1 |
| RAG2 | TRIM15 | FBXO40 | TIGD4 | SCGB2B2 | XXYLT1-AS2 | CTD-2561B21.8 |
| LINGO1 | FGF18 | IL1F10 | VGLL2 | MGAT4D | AC005281.2 | RP11-57A19.2 |
| ZNF385C | TAAR6 | NPTX2 | HOXD4 | OR6C68 | AC098973.2 | RP3-510D11.4 |
| PROB1 | PTCHD2 | PCOLCE2 | FSTL5 | OR6C65 | RP11-4O1.2 | RP1-163M9.8 |
| EBF3 | TMEM179 | IL17RE | HTR1E | OR6C1 | AP001062.7 | RP11-69M1.8 |
| IRF6 | HOXD13 | CLRN1 | OR13J1 | LINC01460 | AC017104.6 | LL22NC03-N95F10.1 |
| IGSF21 | MUC4 | LENEP | OR12D2 | MT1H | AC010907.5 | RP11-411A19.5 |
| SOBP | SCUBE1 | SYNPR | GREM1 | IZUMO3 | AC007099.1 | RP11-148K1.12 |
| ZNF471 | AUNIP | GABBR2 | CCDC178 | OR2J2 | AP000892.4 | CTD-3113P16.11 |
| HEIH | UCN2 | CRYBA2 | MS4A15 | FAM153C | PCOLCE-AS1 | RP11-314A20.2 |
| GNG8 | CYP19A1 | NEK10 | B4GALNT2 | FOXO6 | AP000692.9 | GS1-259H13.2 |
| RTKN | LRRC36 | ADORA1 | TMEM92 | TCEAL5 | ERVMER34-1 | C1QTNF9B-AS1 |
| ZSCAN20 | SEMA6D | SFRP4 | OR51E2 | C2orf72 | CTB-79E8.2 | RP11-485F13.1 |
| ZNF610 | PRSS1 | OR1L4 | MMP26 | CLPSL1 | NKX2-1-AS1 | RP11-206M11.7 |
| TCL6 | FNDC5 | AGR2 | LALBA | NPY4R | AC007383.3 | RP11-306G20.1 |
| RIMS3 | CLDN14 | SLC1A1 | GPD1 | LINC00619 | AC017074.2 | RP3-335E1.1 |
| DCSTAMP | PRRG2 | IGFN1 | GGT6 | TXNDC8 | MIR194-2HG | RP11-119F19.5 |
| SCARA3 | CILP2 | CCKAR | KLK5 | COL5A2 | AC005355.2 | ABC11-4932300O16.1 |
| TEAD1 | LINGO4 | LMOD3 | RCOR2 | TMEM235 | AC104667.3 | RP11-271C24.2 |
| CTGF | TAGLN3 | SGCA | SOAT2 | BTNL2 | AC010731.4 | RP1-221C16.8 |
| VIPR2 | DCHS2 | AK4 | TBX10 | TMEM225 | AC009264.1 | RP11-477N12.5 |
| RASEF | COL8A1 | ETNPPL | PBK | ERICH2 | RNF219-AS1 | RP11-317C20.9 |
| FAM149A | CYP2B6 | CHST3 | OR2B2 | SP5 | AC026471.6 | RP11-231E6.1 |
| NUDT8 | DIO3 | SIX3 | FAM83B | C9orf129 | AC007389.3 | RP11-46A10.5 |
| AVPI1 | STAC | OR3A3 | HIST3H3 | SPANXN5 | AC144450.2 | CTC-281F24.5 |
| HOXB6 | TRIM54 | NPR2 | PTF1A | CSHL1 | AC114730.8 | RP5-1052M9.5 |
| SOST | STRA6 | FOLR1 | FOXI1 | C9orf170 | AC092415.1 | RP13-147D17.3 |
| NPR3 | DDIT4L | CYP2C9 | SCN11A | PRAMEF20 | AC008060.7 | RP11-539L10.5 |
| DLGAP3 | OR2B3 | CNTFR | KLHL30 | PRAMEF15 | ENTPD3-AS1 | CH17-224D4.2 |
| EGFLAM | LRRC3C | APOA5 | GBX2 | AADACL4 | AC011899.9 | RP4-553F17.1 |
| ABLIM2 | PDE11A | TCHH | CHRM1 | C6orf15 | AF230666.2 | RP11-83B20.8 |
| ZNF324B | SENCR | PLA2G4D | GDNF | KRTAP5-11 | AP000295.9 | CTB-26E19.1 |
| TBX2 | SLC13A3 | UPK2 | DPCR1 | FOXB2 | PTGES2-AS1 | RP11-410D17.2 |
| COL6A3 | GPRASP2 | GJD2 | WFDC13 | TRIM31 | AC009948.5 | XXyac-YR21CG7.1 |
| SPR | VGF | CUZD1 | KCTD19 | OR2H2 | AF064858.7 | RP11-630D6.5 |
| SFTPC | RIBC2 | IGF2BP1 | MRAP | TP53TG3D | KRTAP5-AS1 | RP11-446E24.3 |
| ZDHHC9 | LIF | HOXB13 | C7orf33 | VCX3B | AC007620.3 | RP11-30K9.6 |
| NOX4 | CXCL14 | TNNI1 | FRMPD2 | ANKDD1B | CR392000.1 | RP11-572N21.1 |
| FBXW9 | IGLL1 | ADAMTS4 | NPTX1 | KRTAP16-1 | AC007192.6 | RP11-104F15.9 |
| WNT3A | GPX5 | VWA5B1 | KRT20 | TRBV5-5 | AC092301.3 | CTD-2036P10.3 |
| FXYD3 | FCGBP | GPR87 | GLOD5 | TRBV24-1 | AL023875.1 | RP11-685M7.3 |
| CD3EAP | NMB | CALCA | CDK5R2 | TRAV5 | BX004987.4 | RP4-758J24.6 |
| REG1A | FGF17 | PKD1L1 | OR1L6 | TRAV13-2 | AL592183.1 | RP11-121L10.2 |
| SNHG11 | RFPL2 | DNAI1 | DLK2 | TRAV18 | AC008522.1 | RP5-940J5.8 |
| PVT1 | RFPL1 | RIBC1 | NLRP5 | TRAV22 | ST6GALNAC5 | RP4-655C5.9 |
| RYR2 | GAL3ST1 | OR2S2 | OR1N1 | TRAV24 | AC144652.1 | AL122127.25 |
| WNT11 | ADM5 | CCKBR | ANO5 | TRAV26-2 | RP11-1H8.5 | RP11-181C3.1 |
| AHSG | ZNF835 | SFTPA1 | C1orf111 | IGHV2-5 | CTB-96E2.2 | RP3-330M21.5 |
| FAM132B | NKX2-1 | GNRHR | CFAP46 | IGHV4-28 | AL589743.1 | RP11-333B11.1 |
| CBX2 | CDRT15 | ZSWIM5 | ANGPTL7 | IGHV3-35 | RP1-63G5.8 | CTC-241N9.1 |
| DNMT3B | OR9A4 | MPP2 | FAM90A1 | IGHV2-70 | AP000320.7 | RP11-334E6.10 |
| CHMP4C | GOLGA6B | EFNB3 | IFNB1 | IGHV3-73 | AC104389.1 | RP11-677M14.7 |
| RAD54L | OR10Z1 | CYP4A22 | PRND | KRTAP9-6 | AC213203.1 | RP11-712B9.2 |
| CPE | CCDC81 | FGF19 | AQP4 | KRTAP4-9 | AC006130.3 | CTC-366B18.2 |
| SYT5 | G6PC2 | GAS2L2 | CYP4F11 | KRTAP3-3 | AC006126.3 | RP11-171N4.4 |
| SEMA5A | SH2D6 | MSLNL | ANKRD20A4 | KRTAP3-2 | AC000035.3 | ATP5J2-PTCD1 |
| GLYATL2 | SPOCK1 | PRR35 | REG1B | KLK9 | AC010761.6 | CTD-2373H9.3 |
| KIF1A | KL | ITGA11 | OR1A2 | SYT3 | AL354828.2 | NT5C1B-RDH14 |
| FADS6 | PP14571 | CCL16 | LCE1D | CGB8 | AC004623.2 | RP11-613C6.2 |
| PGF | ZNF837 | RCVRN | PRL | CRYGS | AC138430.4 | RP11-83B20.6 |
| LARP6 | ZFP2 | UGT2B10 | OR8U1 | ARHGEF35 | AC124789.1 | RP11-386B13.3 |
| STAB2 | PAMR1 | NEUROG3 | SPTLC3 | TSGA13 | AC011551.3 | RP11-46H11.3 |
| HCN2 | PCDHB16 | CGREF1 | B3GALT1 | LINC00671 | AC137932.4 | RP11-799B12.1 |
| HRAT92 | CCNO | GRIN2C | OR9I1 | TRBV11-1 | AC022154.7 | RP11-170K4.2 |
| PRRG3 | GPR12 | CELF5 | MYOZ2 | TRBV5-3 | AC002985.3 | CTC-480C2.1 |
| TNNI3 | CPB1 | SMR3A | EFCAB3 | TRGV3 | AC011525.4 | RP11-710F7.2 |
| SLC35E4 | STKLD1 | CYP11B1 | OR9G4 | IGLV3-16 | DNAH17-AS1 | RP11-609D21.3 |
| RASD1 | VSTM2L | CHRNB2 | OR5AP2 | MANSC4 | AC074183.3 | RP11-417L19.5 |
| IL17D | RYR3 | HIPK4 | KRT38 | RFPL3S | AF131216.1 | CTD-2526A2.4 |
| CDO1 | NTS | LHCGR | SOSTDC1 | DEFB134 | AC009060.3 | RP11-364L4.3 |
| NRL | OR2A5 | AIRE | CST2 | C1RL-AS1 | AL928768.3 | LA16C-380A1.2 |
| EFNA5 | FST | HSPA4L | CLDN20 | BHLHA9 | CTB-78F1.1 | RP11-286N22.14 |
| GPIHBP1 | ADGRL3 | CCL24 | KISS1 | IFITM5 | AC023491.2 | RP3-522J7.7 |
| GAREM | SKOR2 | MESP1 | COX7B2 | C18orf63 | GS1-25M2.1 | LA16C-312E8.4 |
| BMPER | FAM24A | GBX1 | SIX2 | TMEM211 | AC011366.3 | CTA-992D9.11 |
| ADRA2C | MTNR1B | NELL1 | OR9K2 | SERPINB4 | AL354822.1 | RP11-64J4.7 |
| ANKRD35 | PRR9 | FA2H | FAM71B | SERPINB5 | AP000662.4 | RP4-675C20.4 |
| GPR85 | LOR | VWA2 | SGCD | KRTAP27-1 | RP11-1C8.4 | RP11-654C20.1 |
| IL17RD | CNTN5 | MC3R | RASA4B | CFAP99 | AC008694.3 | CTC-251116.1 |
| KCNJ12 | OR52B2 | AK8 | OR10A3 | GOLGA8O | AF186192.1 | RP13-977J11.3 |
| ANGPTL6 | TKTL2 | TAAR5 | HOXB9 | TCEB3B | AC005943.6 | KB-1125A3.12 |
| SAXO2 | RAET1G | NMBR | GPR37 | DUXAP8 | AC002056.5 | RP11-81A1.4 |
| ZFHX4 | HSD3B2 | VSTM4 | TPD52L3 | PRR23A | AC005753.1 | RP11-95G6.1 |
| RHOD | DUSP26 | EGR4 | SDR16C5 | OR5K3 | OTUD6B-AS1 | RP11-946P6.4 |
| CYP24A1 | RNF212B | AMER2 | DYDC1 | XKR4 | AC008686.1 | CCDC169-SOHLH2 |
| GPR88 | JPH2 | CDX2 | HTRA3 | MIR219A2 | AP000349.2 | RP11-36D19.9 |
| PIGZ | THRSP | MAGEC3 | OR7G3 | DIO1 | AP000687.1 | RP11-475D10.4 |
| PXDN | SLC22A8 | KCNMB4 | PKHD1 | IGKV2D-26 | AC004824.2 | RP11-175K6.2 |
| FXYD2 | HNF1A | SLC16A9 | OR1M1 | IGLV5-52 | AL365181.2 | CTC-321K16.4 |
| PRRT2 | TDO2 | ZNF728 | PLAC1 | IGLV3-27 | AC005578.3 | RP11-166A12.1 |
| SOD3 | ADPRHL1 | CHST5 | HS6ST2 | LCN8 | AL590235.1 | RP11-271K21.12 |
| OR5M11 | MYBPHL | OR1Q1 | PYGO1 | C1orf141 | AL022578.1 | RP11-76C10.4 |
| LYPD6 | GPX6 | IGDCC4 | PATE1 | OR2L5 | AC008103.4 | RP11-394B2.3 |
| IL10 | MAL2 | OCA2 | C8orf74 | CLPSL2 | AC005150.1 | RP11-534L6.5 |
| DUSP15 | OR8B4 | RNF183 | ALK | TOMM20L | AC009060.2 | RP11-1348G14.4 |
| DZIP1 | PSKH2 | OR10C1 | NRTN | HCAR1 | AC022405.1 | CTD-2562J17.6 |
| NCCRP1 | VWA7 | TMEM246 | KCNG3 | FAM163B | AC078882.1 | RP11-124N3.2 |
| HMGA2 | OR4E2 | LRTM2 | RLN3 | IFNL3 | AF127936.7 | CTD-2089N3.2 |
| ZNF620 | SEC14L6 | ASPG | OR2M4 | MAGEA6 | AC012442.5 | RP11-734I18.1 |
| TCF24 | ARMC9 | GABRB3 | PROL1 | OR1J2 | AC006547.8 | RP11-6N13.1 |
| DAO | SCEL | CLEC3A | CSN3 | C17orf102 | HDAC11-AS1 | CTC-490E21.11 |
| TRHDE | SLC34A1 | PLIN1 | SLC10A4 | OR8S1 | AF146191.4 | RP11-119D9.1 |
| RERG | SNTG1 | MRAP2 | CCDC39 | OR51D1 | GS1-24F4.2 | RP11-326C3.11 |
| P4HA2 | OR2M2 | RBPJL | SYCN | OR13G1 | AP001258.4 | RP11-823P9.4 |
| GEM | C3orf56 | TVP23A | AADAT | OGDHL | SCAMP1-AS1 | CTC-458I2.2 |
| BAAT | MAGEA4 | TAF7L | PITX3 | SLC28A3 | AC012005.3 | RP11-574K11.24 |
| SAA2 | ZNF536 | GTSF1L | DKK1 | CCDC160 | ROPN1L-AS1 | KB-1448A5.1 |
| EMP2 | ITGBL1 | GDAP1L1 | CRYBA1 | TTC30A | AC005480.1 | AP000350.10 |
| PLEKHH1 | DEFB118 | GJA10 | RNF43 | OR11L1 | AGPAT4-IT1 | CTD-2283N19.1 |
| CFHR1 | COLCA2 | RRAD | ASIC2 | ENPP1 | AC004009.2 | RP11-521E5.1 |
| AHRR | SRCIN1 | CDH16 | CCL8 | MYH6 | AC130469.1 | CTA-113A6.1 |
| UHRF1 | GSDMC | SLC38A8 | KRT32 | KRTAP26-1 | RP11-6L6.7 | RP11-524F11.3 |
| ERBB3 | ZBED8 | PRTG | RND2 | OR6C74 | AC006386.1 | CTD-2231E14.2 |
| LY6G6C | PTPRD | CRABP1 | CACNG1 | SCGB1D4 | AC010412.1 | RP11-81H3.2 |
| NAV3 | CRP | LMO1 | PHOX2B | C9orf173 | AC105052.2 | RP11-540O11.1 |
| NCKAP5 | BCAN | MSLN | GABRA4 | OR5B17 | CTB-96E2.6 | XX-C00717C00720L.1 |
| SLC44A4 | FBXO36 | CTCFL | ODAM | OR8G1 | AC226118.1 | RP11-101E3.5 |
| EMX1 | GUCY2D | CCL17 | NMU | ADAMTSL2 | AL365202.1 | RP11-1099M24.9 |
| FOXC2 | C2orf16 | RAG1 | ANXA10 | PRB3 | AC124312.1 | CTD-2144E22.9 |
| DLX3 | OR5C1 | MMP15 | NKX3-2 | UGT2B17 | AP002962.1 | RP11-425A23.1 |
| A1CF | MUSTN1 | CPA6 | GYS2 | NRAP | RP11-1C1.5 | CTB-12A17.2 |
| PPP1R1A | OR14K1 | IL36RN | DBX1 | LCE1B | CCDC37-AS1 | RP11-249M12.2 |
| PXDNL | LGI2 | CHRND | P2RX3 | TTC30B | AC005514.2 | CTC-487M23.6 |
| AMHR2 | IRX4 | TERT | KIAA1549L | SLC22A25 | AP000711.1 | AC006486.10 |
| SCGB1C2 | TAS2R9 | CSH1 | SLC1A2 | XRCC2 | AC007326.1 | CTB-138E5.1 |
| FAM92A1 | ADGRA3 | SARDH | ELMOD1 | PRSS48 | AP000437.3 | RP11-893F2.5 |
| WISP2 | CYP1A2 | ICAM5 | ASIC1 | SP6 | AC006128.2 | RP11-394B2.7 |
| ESRG | DMP1 | TMEM174 | TSPAN11 | FAM47B | AP000432.3 | RP11-75C10.6 |
| NPAS3 | GRIA4 | SPZ1 | SYT10 | ZAR1L | AL136419.6 | RP4-539M6.19 |
| MIPOL1 | PLEKHH2 | GPX8 | MYF6 | OR14I1 | AC003005.4 | CTB-46B19.2 |
| RHEBL1 | ZSCAN1 | THEG | MYF5 | NUGGC | JHDM1D-AS1 | RP11-499E14.1 |
| MMACHC | GUCY1A2 | CACNG7 | PRMT8 | LINC00943 | AC000032.2 | RP11-288A5.2 |
| GRM3 | RIT2 | COMP | FGF6 | LINC01164 | AL022344.7 | CTD-2326C4.1 |
| FRMD1 | MAGEA10 | MAG | RASAL1 | FAM150B | AC104534.2 | RP11-700A24.1 |
| KCNN2 | CCDC113 | SLC45A2 | ENDOU | FOXR2 | CTB-61M7.2 | RP5-1054A22.4 |
| RGMA | CACNG2 | HHIP | FGF8 | FAM180A | AC004076.7 | RP11-640L9.1 |
| AJUBA | ZNF843 | CADM4 | PKD2L1 | C6orf222 | AC115522.3 | RP11-269F21.3 |
| ADGRG6 | ZNF670 | TBR1 | LHX3 | ALG1L | RAET1E-AS1 | CTC-384G19.1 |
| OR12D3 | GAL | NPY5R | KCNT1 | TBC1D28 | ATP2A1-AS1 | RP11-436F23.1 |
| ALPK2 | OR4B1 | ASB5 | CBLN1 | PTPRT | CTB-5E10.3 | RP4-742C19.13 |
| THY1 | DRD4 | IL36G | ZP2 | OR6M1 | AC004156.3 | RP11-274A11.4 |
| TRIM29 | SCT | ZIM2 | SALL1 | SPOCK3 | AC002116.7 | RP11-145E17.3 |
| APCDD1L | PTPN3 | WNT2 | TOX3 | C8orf86 | SPACA6P-AS | RP11-360N9.2 |
| TNC | DPP10 | HOXC12 | SLC6A2 | COLCA1 | AC016629.3 | RP11-319G6.1 |
| TUBAL3 | PCSK1 | HOXA13 | RASL12 | OR2C3 | AP000769.7 | RP11-774O3.1 |
| CA5A | FGF10 | MYH14 | FAM189A1 | OR10S1 | AC007950.1 | RP11-624A21.1 |
| ACOX2 | SMPDL3B | CHAD | SCG3 | LINC00523 | AC003973.4 | CTD-2015H6.3 |
| PTPRN | PROP1 | ZFR2 | PDGFRL | GRM7 | CTB-83J4.2 | RP11-483E17.1 |
| LOXL2 | VWA3A | C4BPA | RP1 | NUDT11 | AC007292.3 | RP11-1072C15.6 |
| DSCC1 | GAL3ST3 | DEFB1 | SFRP1 | CRYBA4 | AD000684.2 | RP11-286N22.16 |
| SPINK5 | CAMK2A | WNT10A | JPH1 | GK2 | AC074212.5 | EPPIN-WFDC6 |
| CCDC74B | EPHA8 | USP44 | ANXA13 | ARL9 | AC092071.1 | RP11-514O12.4 |
| SCARA5 | SIX1 | PCDH8 | LHB | CACNA1H | AC100830.3 | CTC-484P3.3 |
| GALNT18 | SPSB4 | SLC30A8 | SLC17A7 | SLC22A6 | AC100830.4 | CTC-506B8.1 |
| FKBP7 | OR4S2 | C8orf48 | RSPH6A | HRNR | AF001548.6 | RP11-622A1.2 |
| MOXD1 | INE2 | LYZL6 | SLC1A6 | ZNF311 | AF001550.7 | RP11-305O6.3 |
| PTPRH | ASIC4 | OR5AR1 | SULT2A1 | OR5D13 | AC139099.4 | CTC-261N6.2 |
| HTR4 | AMZ1 | ESRP1 | HAS1 | DCAF12L1 | RP1-37N7.1 | CTD-2353F22.1 |
| FNDC4 | TSKS | WISP1 | KCNN1 | L1CAM | AC005262.3 | RP11-381K20.2 |
| LHX9 | SEZ6L2 | STMN2 | PON3 | POU3F3 | AC010504.2 | CTB-114C7.4 |
| RGS7BP | PQLC2L | C7orf62 | NPVF | CSAG1 | AC074212.6 | CTC-345K18.2 |
| OR52N2 | BTC | ZFHX2 | ANKRD7 | OR10R2 | ZNF582-AS1 | RP11-394B2.1 |
| DLK1 | BRSK2 | DHDH | FAM188B | LINC01562 | AC005307.3 | RP11-20I23.5 |
| CYGB | HRC | KIF6 | SPAM1 | RBMS3-AS2 | AF067845.1 | RP11-159K7.2 |
| FOXF1 | EIF4E1B | KCNK5 | MOGAT3 | Z99756.1 | RP1-80N2.3 | CTD-2210P15.3 |
| CD300LG | OR51B6 | NACAD | NOBOX | IGBP1-AS1 | AC005546.2 | CTA-243E7.3 |
| GFRA3 | OR4S1 | NEUROD6 | ASPN | OR2L2 | AC002550.5 | CACNA1G-AS1 |
| CFAP74 | CREB3L3 | TBX20 | ELAVL2 | SAMD5 | AC073610.5 | ANKRD33B-AS1 |
| FAM217A | GJC3 | KCNA7 | SLCO1B3 | TEDDM1 | AC131056.3 | RP11-778J15.1 |
| CELA2A | PRR18 | DKKL1 | FGF9 | METTL11B | AP000708.1 | RP11-81A1.7 |
| PLA2G2D | OR4M1 | CNTD2 | CD207 | LINC00222 | AC008984.2 | CTA-212A2.2 |
| SPINK7 | COL11A1 | FABP7 | BCHE | BVES-AS1 | AC145124.2 | CTD-2335A18.2 |
| C1QTNF2 | CDH3 | GRIK2 | SERPINI2 | RIPPLY2 | AC156455.1 | RP11-561O23.9 |
| MUC5B | DMRT3 | GDF9 | HHLA2 | KHDC3L | AP000721.4 | RP11-165I9.10 |
| UGT3A1 | ABCC9 | COLEC12 | UPK1B | DPPA5 | AC011718.2 | CTD-2175A23.1 |
| NKD2 | OR4D11 | GUCA1C | PEX5L | SPANXN1 | AC113404.1 | RP11-495K9.6 |
| CABS1 | MC5R | ACSS3 | CCL20 | SPANXA2 | AC022182.1 | RP11-548K23.11 |
| PADI1 | AIM1L | TPBG | TFCP2L1 | PNMA5 | AC007325.1 | RP11-359P5.1 |
| SLC26A1 | SNAP91 | TPSG1 | GCG | GJC2 | RP1-65P5.5 | RP11-1086I4.2 |
| SLIT2 | ASCL3 | OPRD1 | TACR1 | OR5H2 | CTB-32O4.3 | LLNLR-304A6.1 |
| PLPPR4 | KLHL38 | FAM135B | DNAH6 | RASSF9 | AP006621.8 | RP13-977J11.6 |
| MYOM3 | SYT1 | PRG4 | SMYD1 | GOLGA6L9 | AC142472.6 | RP11-148B6.2 |
| SCN2B | ATP2B3 | DMRTC2 | SLC9A2 | SPANXA1 | AC005618.6 | RP11-156P1.2 |
| AADAC | FGFR3 | SLC26A7 | SLC5A7 | ZNF667 | AC008592.8 | CTD-2013N17.7 |
| FAM57B | ETV4 | PMP2 | LCT | POTEH | AL161668.5 | RP11-434D12.1 |
| GPR61 | CRMP1 | CRH | GRIN3B | AKR1B10 | RP6-74O6.6 | RP11-33B1.4 |
| TTLL8 | TRAM1L1 | EQTN | FAM184A | CYP2A7 | AL133245.2 | RP11-77K12.7 |
| NAT2 | NPAS4 | OLFML3 | MARK1 | KRTAP9-9 | AC068831.6 | RP11-220I1.5 |
| SLC38A4 | FLRT3 | HOXB5 | MORN1 | KRTAP4-6 | ARL2-SNX15 | AC005606.15 |
| ADCY8 | SRPX2 | CCNB3 | NPHS2 | TMPRSS11F | TMEM51-AS1 | RP5-1142A6.9 |
| FMOD | GPR148 | AKAP4 | PDC | OR2T6 | AC073072.5 | RP11-83N9.5 |
| LUM | OR10K1 | IL19 | PRAMEF12 | SOWAHC | AP000679.2 | RP11-483P21.2 |
| PDZD9 | DMRT2 | ASB15 | AMPD1 | NPIPB6 | AL021546.6 | CTD-2616J11.9 |
| KERA | OSBPL6 | FERD3L | TNNI3K | CSF2RA | AC062028.1 | RP11-384M15.3 |
| AMDHD1 | ADCY5 | NTN5 | CFHR3 | FKBP1C | AC012593.1 | CTD-2620I22.3 |
| OR4K1 | BARX1 | TFAP2E | NT5C1A | OR5B21 | AF178030.2 | CTD-237103.3 |
| PROM2 | CA6 | TAAR8 | MFAP2 | RTP2 | AC105760.2 | CTC-273B12.7 |
| OBP2A | COL5A3 | RSPO3 | PTCH2 | B3GNT6 | SLC2A1-AS1 | RP11-1035H13.2 |
| DKK2 | CPB2 | VAX2 | MROH9 | SLC34A3 | AP001046.6 | CTD-3203P2.1 |
| EME1 | DAB1 | SHC3 | PLPPR5 | RD3 | AC016582.2 | CTD-3073N11.9 |
| DHH | LVRN | MAPK4 | CHRNB4 | AKR1C4 | AC102953.4 | RP11-753A21.2 |
| WNT7A | PPP1R1B | KCNIP3 | RBP2 | ZNF534 | AC098823.3 | RP11-830F9.5 |
| NCAM2 | OVOL1 | UNC45B | C9 | FAM3D | AC093375.1 | CTB-33G10.6 |
| CHODL | FSHB | SLC30A3 | IL5 | OR10G6 | AC104655.3 | LLNLR-249E10.1 |
| CDH24 | USP29 | GPR31 | SLC27A6 | MAGEA1 | AC016999.2 | CTB-60B18.10 |
| HCRTR1 | PKP1 | SYT8 | GABRR2 | ABCA4 | AC105053.3 | RP4-806M20.4 |
| ALX3 | OR1S1 | SAA4 | TULP1 | LRRTM3 | AC003986.5 | RP11-553L6.5 |
| IL17F | LIN28A | ACADL | GLP1R | CSF2RA | AC079776.1 | RP11-382A20.4 |
| SLC28A1 | PGR | KIF2B | BMP5 | EVX1 | AC007879.7 | CTD-2207O23.10 |
| KLHL40 | KCNK2 | LRRC4C | RBM24 | ZCWPW2 | AC013460.1 | RP11-158H5.7 |
| TRIM63 | OPRK1 | SLC5A12 | CAP2 | FGF11 | AC018462.2 | RP5-1085F17.3 |
| SLC30A2 | SEMA5B | HMCN2 | FHL5 | EYA1 | STEAP3-AS1 | RP4-569M23.5 |
| DPYSL5 | GRHL2 | GAS2 | SIM1 | HOXA5 | AC099684.1 | CTC-444N24.6 |
| ACAN | INSM1 | ASXL3 | BVES | NTRK2 | AC007163.6 | RP11-20G6.3 |
| TMED6 | GJA9 | PLEKHS1 | COL9A1 | COL15A1 | AC124057.5 | RP11-834C11.11 |
| OCM | EVC | GPR1-AS | EYA4 | POM121L12 | ARMCX3-AS1 | RP11-923I11.4 |
| APOBEC1 | CAMSAP3 | VAX1 | NR2E1 | SMOC1 | AC106873.4 | RP11-219B4.7 |
| NAA11 | CYP2W1 | HABP2 | UNC93A | RTBDN | AC005682.5 | RP11-1136J12.1 |
| SHCBP1L | KIRREL2 | KISS1R | PACRG | CPN2 | AC097381.1 | RP11-356C4.3 |
| NANOG | NOTCH3 | RGR | C6orf118 | HOXD11 | AC133528.2 | RP11-1006G14.4 |
| PRM2 | CDHR2 | SLC25A2 | SMOC2 | LINC00174 | AC020571.3 | RP11-63M22.1 |
| CDKL2 | TTC9B | CARD14 | PRPH2 | NOX1 | AC069513.4 | RP11-615I2.2 |
| SHROOM3 | CA12 | PCDHB10 | WISP3 | HTR1D | AC131097.4 | CTD-2369P2.8 |
| RFX4 | CACNG5 | HOXB8 | TBX18 | SPAG6 | AC091801.1 | CTD-3252C9.2 |
| UNC5D | PROZ | PANO1 | PCDHB2 | TYR | AC093802.1 | RP5-1052I5.2 |
| LRFN2 | TTLL9 | DMRTB1 | NUDT12 | ITGA8 | KRTAP10-10 | CTD-2528L19.4 |
| HS3ST2 | ZNF80 | IGSF11 | HAND1 | FAP | AC106876.2 | RP5-1142A6.7 |
| OPRM1 | ACTL6B | ZC2HC1B | PCDHB5 | ADCY2 | AC005481.5 | RP11-375I20.6 |
| SLC4A9 | COX4I2 | ADGB | PCDHB6 | AC011239.1 | AC091878.1 | RP11-303E16.8 |
| CDH9 | C1QL1 | SLC44A3 | PCDHB7 | RASAL2-AS1 | AC023469.1 | RP11-373L24.1 |
| TEX14 | FKBP6 | KCNA10 | HAVCR1 | AC068282.3 | SRGAP3-AS1 | AC008746.12 |
| GABRA2 | RAX2 | LRTM1 | GRM6 | DNAJC9-AS1 | TMEM72-AS1 | RP11-77H9.2 |
| RHCG | GDF5 | SHISA2 | IL12B | AC007750.5 | AC114812.8 | RP11-24M17.4 |
| SERP2 | WFIKKN2 | March4 | CDH6 | AC007563.5 | COL4A2-AS2 | AC018755.17 |
| STC2 | DEFB129 | STBD1 | IRG1 | AC114783.1 | CDKN2A-AS1 | RP11-388M20.6 |
| ST8SIA2 | SPATA3 | ILDR2 | HTR2A | AC007386.2 | AC015936.3 | RP11-316M20.1 |
| SEPT12 | LRRN4 | ELOVL3 | HES2 | AC073834.3 | AC007250.4 | AC006116.24 |
| KCTD14 | GFRA4 | UNC80 | ALX4 | AL035610.2 | AC091814.2 | RP11-111J6.2 |
| ALLC | PHF21B | GDA | CCDC85A | NEURL1-AS1 | AP001055.6 | CTD-2130O13.1 |
| KCNA6 | ACOT12 | FAM171B | GYG2 | SAPCD1-AS1 | RP11-3B7.1 | RP11-420N3.2 |
| FREM2 | TAS2R5 | SCN1A | LAMC2 | AC007405.8 | AC104134.2 | RP11-254F19.3 |
| C2orf50 | TAS2R16 | ZC2HC1C | LZTS1 | AC064853.2 | AP001626.1 | RUNDC3A-AS1 |
| PIH1D2 | TFAP2B | PVRL4 | WNT8A | AC079988.3 | AC099850.1 | RP11-718I15.1 |
| PPP1R1C | CDH4 | ANKRD53 | ARSF | KCNMA1-AS1 | AC008069.1 | AC006116.20 |
| CNDP1 | OPN1SW | VSX2 | LTK | AC079922.3 | EFCAB6-AS1 | RP11-264B17.4 |
| GLT8D2 | CYP11B2 | SYT2 | NGFR | PSG6 | AP001469.5 | RP11-108P20.3 |
| OR5M9 | HES7 | PPP4R4 | EYA2 | TRBV7-4 | AC002456.2 | CTD-2013N17.4 |
| MYOCD | HOXD3 | LEFTY2 | POU1F1 | CEMIP | LA16C-OS12.2 | RP5-1024G6.7 |
| MKX | HOXD10 | ACTA1 | CTNNA2 | CABYR | CTC-518B2.8 | RP5-1142A6.5 |
| BEND6 | CCDC89 | FLG | CLDN18 | ACTL7B | RP5-1172N10.4 | CTC-273B12.10 |
| AGXT2 | KLHDC7A | FLG2 | ADAM7 | KRTAP5-9 | RP11-394O4.5 | RP11-605F22.1 |
| DUOX2 | GIPR | CYP4B1 | GUCY2C | H1FOO | RP11-463C8.7 | RP11-345P4.7 |
| ABCA9 | SYT7 | DMRTA2 | AFM | NPC1L1 | RP11-109D20.2 | RP11-307C19.2 |
| CERS3 | EXD1 | GABRA6 | CNGB1 | CPNE7 | RP4-621N11.2 | RP11-253M7.1 |
| PANX3 | LGALS7B | LHX1 | CHAT | ADAM11 | CTC-339O9.1 | RP11-279F6.2 |
| KCNJ16 | FOXE1 | KAAG1 | MS4A12 | CACNG4 | GLTSCR2-AS1 | RP11-540O11.7 |
| PDE8B | TMEM139 | RGS4 | MYO3B | GPC4 | RP11-654A16.3 | XXbac-BPG300A18.13 |
| ADGRF1 | UCHL1 | DCDC2 | SLC6A15 | | | TM4SF19-TCTEX1D2 |

### Example 2: Enrichment of Tissue-Derived Cf-mRNA by Size Fractionation

Generally, most RNA in blood is found within blood cells. Methods for preparing serum and plasma generally involve a low-speed spin that removes most blood cells. However, residual blood cells are a significant source of noise that may interfere with an analysis of cf-RNA.

The GTEx tissue expression database was used to identify tissue-specific and organ-specific genes. A gene is considered tissue or organ specific if its expression is at least five-fold higher is one tissue or organ compared to all other tissues and organs. Tissue-specific and organ-specific genes detected in this study are presented in Tables 2-7.

TABLE 2
Tissue-specific genes (455) detected in cell-free mRNA

| | | | | | | |
|---|---|---|---|---|---|---|
| DBNDD1 | GCK | HAPLN2 | JPH3 | HSD11B1L | TH | FAM24A |
| PON1 | AMBP | BEX1 | NRGN | KLK1 | MYLPF | HSD3B1 |
| ABCC8 | APBA1 | SAA2 | UCHL1 | KLK4 | SLC9A4 | HSD3B2 |
| ETV1 | SETP4 | CABLES1 | PSD3 | KLK2 | PRNT | BTBD17 |
| PAX6 | PPY | DSG1 | CXCL13 | KLK6 | CYP8B1 | LY6G6C |
| FMO3 | VTN | B4GALNT1 | FBXO43 | KRT80 | OXTR | MOG |
| IYD | CDR2L | STAB2 | GLYATL2 | MGAT5B | GPR62 | PRSS1 |
| PRSS3 | HPX | RAPGEF5 | NMNAT2 | SOST | SPEM1 | MUC12 |
| CPS1 | APOA4 | BAAT | ATP2B2 | SLC22A11 | TMIE | CYS1 |
| HSD17B6 | APOC3 | SLC22A7 | SLC6A1 | FAM107A | GPR88 | DOK6 |
| CYP46A1 | PTPN5 | CAPN11 | SYN2 | MOBP | ACTL9 | LRRC30 |
| RAD51 | IL23A | FXYD2 | CCNB2 | SFTPC | AP003774.4 | TSSK1B |
| MCF2L2 | GSG1 | CYP2C8 | FAM81A | PHYHIP | NLRP10 | PRR22 |
| PTPRN | MGP | RBP4 | CABP1 | ADAM18 | KCNK4 | CTAGE1 |
| ITIH1 | ACRBP | PPFIA2 | HPD | CTRB2 | BGN | KRT222 |
| CAMK2B | PDE10A | FOXN4 | FGF17 | CPLX1 | SLC35D3 | CEACAM18 |
| RIMBP2 | KNG1 | SLC25A47 | APOA2 | KCNK9 | GALNT9 | GOLGA6B |
| SLC6A16 | HRG | CYP11A1 | LRRC36 | LINGO1 | C2CD4C | AC007249.3 |
| DLX3 | CSPG5 | MYLK3 | FAM131B | MUC17 | ACSM5 | C2orf16 |
| SPA17 | REG1A | ADAMTS18 | CILP2 | SERPINA6 | 11-Mar | POM121L12 |
| BCAS1 | TPO | ADAD2 | FTCD | USP50 | COLEC10 | MYBPHL |
| ROPN1 | PROC | LRRC46 | SPTBN4 | FABP4 | KCND2 | CDRT15 |
| NGEF | DLGAP3 | ADCYAP1 | COX6B2 | VSTM2A | C17orf74 | HBD |
| MOV10L1 | RIMS3 | FEM1A | NLRP4 | SPATA24 | PRSS55 | FAM95B1 |
| CD5L | ACTL8 | CACNG8 | CYP3A7 | DLGAP1 | RALYL | AC005076.5 |
| PPP2R2C | IGSF21 | KLK3 | LY6K | GTSF1 | LCN12 | AC079767.4 |
| SNCB | SERPINC1 | FAM71E1 | PGLYRP2 | RBMXL2 | ANKS1B | PRKAR2A-AS1 |
| PAX2 | APOA1 | MMEL1 | CCDC116 | PGK2 | ZNF479 | GPX5 |
| RGS11 | ZNF541 | FCN3 | DMKN | MUC7 | MC2R | RP1-90J20.8 |
| MAP2 | SLC8A2 | BARHL2 | IP6K3 | TPPP | TEX33 | KCNIP2-AS1 |
| PKD2L2 | TNNT2 | ITGA10 | HS3ST6 | APLN | SFTPA2 | LINC00710 |
| RIMS1 | TTR | RGS5 | ITIH3 | KSR2 | SPATA8 | ATXN8OS |
| RDH8 | CRHR1 | NR1I3 | FBLIM1 | FGG | NRG3 | CYP21A2 |
| GCKR | IQSEC3 | ECM1 | CAMK2N1 | FGA | TEX38 | HLA-DQB2 |
| CETP | A1BG | OAZ3 | LRRC52 | FGB | UBL4B | RP11-1212A22.4 |
| MT3 | GHSR | CRNN | C1orf145 | PAH | KDM4D | LINC00264 |
| PHACTR3 | HPCA | REG3G | LRRTM1 | UTF1 | MYT1L | APOC2 |
| ARHGAP28 | AKR1D1 | PKP4 | VSNL1 | WDR87 | PDZD7 | AC004448.5 |
| MYO15A | C4BPB | COL8A1 | TEKT4 | KLF17 | PNLIPRP1 | RP11-343J3.2 |
| APOH | WFDC3 | TAGLN3 | PDHA2 | GAP43 | SBK2 | CLEC2L |
| HCN2 | SLC12A5 | AHSG | SPRR2D | ARNT2 | ERC2 | PRMT5-AS1 |
| ACR | CHST8 | GC | TDRD10 | IL17D | ADH1A | AC003075.4 |
| CHADL | IRGC | CDCA5 | EFHB | AGXT | SMTNL2 | TEX35 |
| DHRS2 | GRM4 | NXF3 | CADPS | CBX2 | PRR19 | SMKR1 |
| CPNE6 | TCP11 | ARHGAP36 | ALB | GOLGA6L2 | FBLL1 | RP11-758P17.3 |
| HRH3 | LAMP5 | MAGEA4 | DNAJC5G | ASPHD1 | APOD | PNMA2 |
| CCM2L | PRDM7 | PXDNL | CLDN19 | ZIC4 | KRT77 | SPRR2A |
| EPPIN | AUNIP | DNAJC5B | CDC25A | CA5A | GJB4 | TIPARP-AS1 |
| TNNC2 | IGLL1 | ACTL7B | CAMKV | CADM2 | THEM5 | CFHR1 |
| CELF4 | RIBC2 | DRD2 | HIST1H2AA | INHBC | LINC00176 | OIP5-AS1 |
| CDH20 | VGF | SLC22A8 | SLC35G3 | TSGA10IP | SCN8A | HULC |
| LIPG | ARHGAP22 | DUSP15 | ADCY1 | UBQLNL | ZNF165 | AP003068.9 |
| PRSS54 | SYT5 | C20orf144 | BAALC | UBQLN3 | CYP2B6 | CYP2A6 |
| ACSBG1 | TNNI3 | TKTL2 | NKX6-3 | PFN4 | SLC22A12 | TMPO-AS1 |
| CKM | KIF1A | MAT1A | TMEM52B | LY6H | TUBA3C | RP11-1100L3.7 |
| CLEC4M | UNC13A | NPAS3 | SERPINA12 | ACER2 | AVPR1B | RP11-219B4.5 |
| IL4I1 | CYP2E1 | TDO2 | LIPC | SPACA4 | ADH4 | RP11-14J7.6 |
| PRX | PRRG1 | TMEM178A | SPIC | HTR1A | TMEM239 | PRC1-AS1 |
| EBI3 | GFAP | SPOCK1 | TMEM100 | PSAPL1 | ITGBL1 | TUBB3 |
| ZNRF4 | SLC34A1 | GRM1 | SAMD14 | C5orf64 | RYR2 | TMEM179 |
| CCDC114 | TUBG1 | RAB3C | GPRC5B | ALOXE3 | TAT | RPPH1 |
| PLEKHA4 | G6PC | CPB1 | LPO | GPC5 | GRM3 | SLC35G6 |
| RAB3A | REEP2 | FAM81B | LINC00905 | TPPP2 | NOS1AP | CTD-2184D3.6 |
| ETV2 | BCAN | CABYR | TTYH1 | PIPOX | LOR | RP11-505K9.4 |
| PTPRZ1 | CRP | THY1 | DNAAF3 | NKPD1 | PRR9 | RP3-422G23.4 |
| MARCH11 | SEPT4 | | | | | |

TABLE 3
Red blood cell-specific genes (23) detected in cell-free mRNA

| | | | | | |
|---|---|---|---|---|---|
| SPTA1 | ALAS2 | TRIM10 | ANKLE1 | SLC6A9 | ABCB10 |
| CENPF | SPTB | HMBS | AHSP | RHCE | YPEL4 |
| ANK1 | FHDC1 | HBD | TRAK2 | ATP1B2 | CA1 |
| EPB42 | ACHE | ACSL6 | MICALCL | IFIT1B | |

TABLE 4
Platelet-specific genes (326) detected in cell-free mRNA

| | | | | | | |
|---|---|---|---|---|---|---|
| ITGA2B | LY6G6F | P2RX1 | FKBP1B | RSU1 | ITGB3BP | LAMTOR1 |
| ITGB3 | GP6 | PPBP | HOMER2 | ADCY3 | PKM | GP1BB |
| F13A1 | MFSD1 | ST3GAL3 | TM6SF1 | DAPP1 | OST4 | PRKAR1B |
| JAM3 | 43713 | SCFD2 | PITPNM2 | PF4V1 | LYPLAL1 | MAPRE2 |
| TREML1 | TSPAN33 | PTCRA | GLA | CLCN3 | F11R | BET1 |
| CLU | RAB27B | ENDOD1 | TTC7B | PLA2G12A | LRP12 | SH3BGRL3 |
| C6orf25 | GNG11 | TDRP | PDGFA | SLC10A3 | DPY19L1 | SLC2A3 |
| PROS1 | MFAP3L | MLH3 | SNN | STX1A | CCR4 | CD84 |
| ABCC3 | CD151 | TNFSF4 | PRUNE | MAX | PYGB | TGFB1 |
| THBS1 | SLC6A4 | LRRC8B | TLR5 | GAS2L1 | MYZAP | TPST2 |
| CD9 | VEPH1 | PARVB | TM2D2 | PDLIM1 | ARMCX6 | BCAP31 |
| SLC24A3 | GNB5 | C2orf88 | ANKRD28 | RHOBTB1 | RTN2 | TST |
| MPL | GP5 | NEXN | CPNE5 | CCL5 | TLK1 | DAB2 |
| AQP10 | IGF2BP3 | CLEC1B | ARHGAP18 | MYLK | TVP23B | TIMP1 |
| TNNC2 | PKHD1L1 | KIF2A | FERMT3 | MAP1A | NCK2 | DAD1 |
| SELP | SPARC | PANX1 | MEIS1 | C1orf198 | DSE | PTPN12 |
| ELOVL7 | HGD | SLA2 | SLC39A3 | RGS10 | TMEM104 | MOB1B |
| CFAP161 | LTBP1 | ARHGAP6 | TAGLN2 | SNAP23 | C12orf75 | GFI1B |
| BEND2 | SLC18A2 | PDE3A | TPM1 | ASAP2 | MTURN | ITM2B |
| SPX | ZNF185 | LRRC8D | SDC4 | TGFB1I1 | PPP1R14A | ODC1 |
| CABP5 | LIMS1 | F2R | COL24A1 | ARL6IP5 | MGLL | LIPC |
| ZNF385D | GP9 | TMEM40 | TBXA2R | ST7 | LAT | CDIP1 |
| MMRN1 | MYL9 | ENO2 | PTPRJ | TRAPPC1 | PLXDC2 | FAXDC2 |
| RGS18 | RUFY1 | PVALB | RNF208 | FAM63A | TTC39B | DGKG |
| CD226 | LIPA | PF4 | TMEM189 | RAB6B | CARD19 | RAB32 |
| PRKAR2B | ACRBP | NGFRAP1 | CDC14B | RABGAP1L | KIF3C | ASAH1 |
| VIL1 | PCSK6 | TPP1 | ACVR1 | CDKN1A | MEST | OPHN1 |
| GNAZ | GRAP2 | PRTFDC1 | SMOX | SLC9A9 | CLIP2 | MTFR1L |
| ENKUR | MAP3K7CL | CA13 | FHL1 | MGAT4B | SLC50A1 | YPEL5 |
| EGF | ANGPT1 | EFHC2 | CTDSPL | 43526 | GUCY1A3 | TTC33 |
| SH3BGRL2 | P2RY12 | SAMD14 | MED12L | GPX1 | WDR44 | CA2 |
| TUBB1 | RGS6 | LGALSL | TMEM91 | CD27-AS1 | PROSER2 | ARHGAP21 |
| PGRMC1 | DNM3 | SLC8A3 | MSANTD3 | CNST | CRAT | SLC37A1 |
| GUCY1B3 | TSPAN9 | NT5M | SUSD1 | ACTR3B | CAMTA1 | THRB |
| SDPR | CXCL5 | CYB5R1 | ATP2A3 | TUBA4A | TPM4 | LPAR5 |
| PTGS1 | SIAE | PRR7 | TWSG1 | LDLRAP1 | TMEM55A | SH3TC2 |
| GP1BA | TMEM185A | AP001189.4 | CCDC92 | TAX1BP3 | TSPAN18 | IFRD1 |
| CTSA | RDH11 | AGPAT1 | TSC22D1 | ADAM9 | CLDN5 | TMEM106C |
| CMTM5 | NPTN | LEPROT | SSX2IP | RAP1B | C1orf116 | B2M |
| ALOX12 | C19orf33 | WRB | MCUR1 | RAB37 | SPOCD1 | BZRAP1-AS1 |
| PDE5A | FSTL1 | SMIM3 | YIF1B | PARD3 | DYNLRB1 | RP11-290H9.2 |
| ANO6 | CTTN | TPTEP1 | NRGN | TMEM50A | PTPRS | RP11-501J20.5 |
| ITGB5 | RBPMS2 | NOMO1 | PEAR1 | TTYH3 | EHD3 | RP11-544M22.13 |
| C15orf54 | SPINT2 | PCYT1B | TRAPPC2 | UBL4A | YWHAZ | MSANTD3-TMEFF1 |
| VCL | MMD | NT5C3A | BMP6 | CDK2AP1 | SRC | SEPT5 |
| TUBA8 | ESAM | AIG1 | HACD4 | ILK | GRK5 | MARCH2 |
| ITGB1 | WASF3 | SEMA4D | RAB11A | LGALS12 | YWHAH | |

TABLE 5
Neutrophil-specific genes (239) detected in cell-free mRNA

| | | | | | | |
|---|---|---|---|---|---|---|
| GOS2 | ANPEP | ITPRIP | BASP1 | IL1RAP | CEACAM3 | N4BP1 |
| PTGS2 | KRT23 | PILRA | MYADM | ST8SIA4 | FRAT1 | MSRB1 |
| CXCL1 | SIRPA | CPD | TNFRSF1A | MSL1 | RHOB | IPMK |
| EGR1 | CXCR4 | FCGR2A | SGK1 | IFNGR2 | IL4R | APOBEC3A |
| CXCL8 | FPR2 | IGF2R | AQP9 | NINJ1 | PLBD1 | NR4A1 |
| DUSP1 | BTG2 | HSPA6 | CD14 | APOBR | DYSF | ALOX5 |
| JUNB | NAMPT | THBD | HRH2 | CXCL16 | DHX34 | ZFP36L1 |
| FOS | CEBPB | TCIRG1 | OGFRL1 | RASGRP4 | SIGLEC10 | MBD6 |
| MMP25 | ZDHHC18 | TLR2 | ACSL1 | FGL2 | IGSF6 | NCF2 |
| RGS2 | IER3 | KDM6B | MPPE1 | NRBF2 | CCNJL | SIGLEC9 |
| TREM1 | PLPPR2 | CFD | CTBS | DPEP2 | BST1 | LILRB2 |
| PLAUR | PANX2 | CREB5 | AMICA1 | BCL6 | TLE3 | B3GNT8 |
| ZFP36 | LRRC4 | PLXNC1 | IL17RA | CHSY1 | VMP1 | JUN |
| MME | SLC19A1 | FCGR3B | NFAM1 | GRN | PDE4B | UBE2D1 |
| IER2 | ADGRE2 | LILRA2 | TNFRSF1B | PPP1R15A | CR1 | PADI2 |
| CSF3R | ATG16L2 | CEBPD | FFAR2 | LAT2 | RARA-AS1 | ABTB1 |
| SOCS3 | CXCR2 | P2RY13 | ABCA7 | TECPR2 | RPGR | MNDA |
| ITGAX | IL6R | MMP9 | MXD1 | ZNF467 | ATP6V0B | RARA |
| DGAT2 | NFIL3 | SLC16A3 | MEGF9 | ARSA | CPPED1 | IMPDH1 |
| MGAM | SORL1 | XKR8 | STX3 | WDFY3 | METRNL | PLK3 |
| C5AR1 | FPR1 | NFKBIA | RBM47 | DAPK2 | RELT | BCL3 |
| CSRNP1 | QPCT | FCGRT | CLEC4E | NOTCH2 | LIMK2 | TLR4 |
| ADGRE3 | GLT1D1 | CXCR1 | LRRC25 | PISD | NABP1 | C16orf54 |
| MBOAT7 | ATHL1 | TLR1 | PHC2 | EEPD1 | C15orf39 | IMPA2 |
| BCL2A1 | RNF149 | KREMEN1 | C10orf54 | PELI1 | PFKFB4 | CYB5R4 |
| CSF2RB | SLC11A1 | FAM129A | XPO6 | CD46 | NCF4 | MID1IP1 |
| LILRB3 | SEMA4A | PTAFR | TUBA1A | NUAK2 | IL1RN | RAB3D |
| ALPL | IL1B | CHST15 | ADGRE5 | IFNGR1 | CCR1 | LYN |
| PROK2 | WLS | TMEM154 | LAMP2 | SELL | TSEN34 | KIAA1551 |
| PI3 | LRRK2 | CMTM2 | GNG10 | IL13RA1 | TMCC3 | RNF130 |
| EGR2 | ADM | RNASET2 | MEFV | ANKRD13D | GK | C7orf43 |
| ADAM8 | SLC43A2 | NOTCH1 | MPZL3 | FRAT2 | HCK | CTC-250I14.6 |
| VNN2 | SLC12A9 | AATK | NLRP12 | SECTM1 | TMEM127 | TNFRSF10C |
| TRIB1 | KCNJ15 | TNFAIP2 | SELPLG | TMEM43 | RNF19B | TMEM120A |
| SULF2 | | | | | | |

TABLE 6
Brain-specific genes (163) detected in cell-free mRNA

| | | | | | | |
|---|---|---|---|---|---|---|
| DBNDD1 | CDH20 | ARHGAP22 | SPOCK1 | BAALC | CADM2 | AVPR1B |
| ABCC8 | ACSBG1 | SYT5 | GRM1 | NKX6-3 | LY6H | GRM3 |
| ETV1 | RAB3A | KIF1A | RAB3C | SAMD14 | HTR1A | NOS1AP |
| PAX6 | PTPRZ1 | UNC13A | THY1 | GPRC5B | C5orf64 | BTBD17 |
| CYP46A1 | GCK | PRRG1 | JPH3 | TTYH1 | GPC5 | MOG |
| MCF2L2 | APBA1 | GFAP | NRGN | HSD11B1L | TH | DOK6 |
| PTPRN | SEPT4 | REEP2 | UCHL1 | KLK6 | OXTR | KRT222 |
| CAMK2B | CDR2L | BCAN | PSD3 | MGAT5B | GPR62 | ATXN8OS |
| RIMBP2 | PTPN5 | HAPLN2 | NMNAT2 | FAM107A | TMIE | RP11-1212A22.4 |
| BCAS1 | PDE10A | BEX1 | ATP2B2 | MOBP | GPR88 | AC004448.5 |
| NGEF | CSPG5 | CABLES1 | SLC6A1 | PHYHIP | KCNK4 | RP11-343J3.2 |
| PPP2R2C | DLGAP3 | B4GALNT1 | SYN2 | CPLX1 | SLC35D3 | CLEC2L |
| SNCB | RIMS3 | RAPGEF5 | FAM81A | KCNK9 | GALNT9 | PNMA2 |
| RGS11 | IGSF21 | PPFIA2 | CABP1 | LINGO1 | C2CD4C | OIP5-AS1 |
| MAP2 | SLC8A2 | ADAMTS18 | FGF17 | VSTM2A | KCND2 | TUBB3 |
| RIMS1 | CRHR1 | ADCYAP1 | FAM131B | DLGAP1 | RALYL | TMEM179 |
| MT3 | IQSEC3 | CACNG8 | SPTBN4 | TPPP | ANKS1B | RPPH1 |
| PHACTR3 | GHSR | BARHL2 | CAMK2N1 | APLN | NRG3 | CTD-2184D3.6 |
| MYO15A | HPCA | PKP4 | C1orf145 | KSR2 | MYT1L | AP003774.4 |
| HCN2 | SLC12A5 | TAGLN3 | LRRTM1 | GAP43 | PDZD7 | |
| CHADL | CHST8 | ARHGAP36 | VSNL1 | ARNT2 | ERC2 | |
| CPNE6 | GRM4 | DRD2 | CADPS | IL17D | FBLL1 | |

TABLE 7
Liver-specific genes (63) detected in cell-free mRNA

| | | | | | | |
|---|---|---|---|---|---|---|
| PON1 | VTN | A1BG | CYP2C8 | APOA2 | FGA | COLEC10 |
| FMO3 | HPX | AKR1D1 | RBP4 | FTCD | FGB | ADH1A |
| CPS1 | APOC3 | C4BPB | SLC25A47 | CYP3A7 | PAH | CYP2B6 |
| HSD17B6 | KNG1 | CYP2E1 | NR1I3 | PGLYRP2 | AGXT | ADH4 |
| ITIH1 | HRG | G6PC | AHSG | ITIH3 | CA5A | TAT |
| GCKR | PROC | CRP | GC | ALB | INHBC | APOC2 |
| APOH | SERPINC1 | SAA2 | MAT1A | UPC | PIPOX | CFHR1 |
| CLEC4M | APOA1 | BAAT | TDO2 | SERPINA6 | CYP8B1 | HULC |
| AMBP | TTR | SLC22A7 | HPD | FGG | ACSM5 | CYP2A6 |

Tables 8 and 9 present genes of interest for the diagnosis of liver-specific disease and pregnancy that to not meet the stringent criteria for non-blood genes.

TABLE 8
Additional liver-specific genes detected in cell-free mRNA

| | | | |
|---|---|---|---|
| MBOAT7 | PPP1R3B | PNPLA3 | TM6SF2 |

TABLE 9
Pregnancy associated genes detected in cell-free mRNA

| | | | | | | |
|---|---|---|---|---|---|---|
| ALPP | CAPN6 | CGA | CGB | CSHL1 | LGALS14 | PAPPA |
| FABP1 | FGA | FGB | ITIH2 | KNG1 | OTC | SLC38A4 |
| PLAC4 | PSG7 | ADAM12 | CSH1 | GH2 | | |

### Example 2: Enrichment of Tissue-Derived Cf-mRNA by Size Fractionation

Generally, most RNA in blood is found within blood cells. Methods for preparing serum and plasma generally involve a low-speed spin that removes most blood cells. However, residual blood cells are a significant source of noise that may interfere with an analysis of cf-RNA.

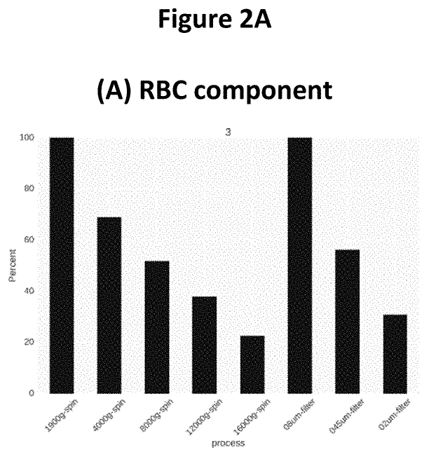

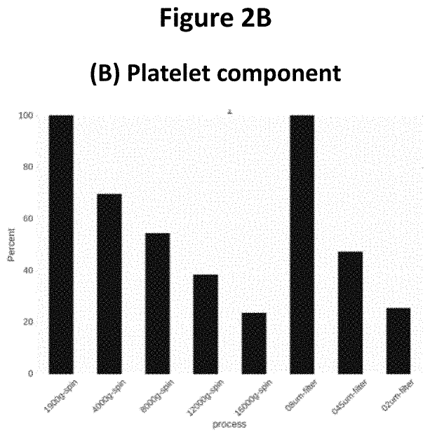

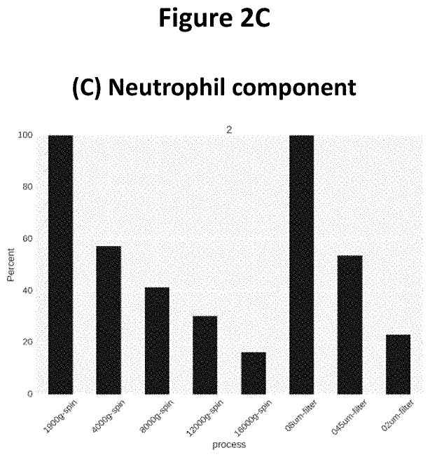

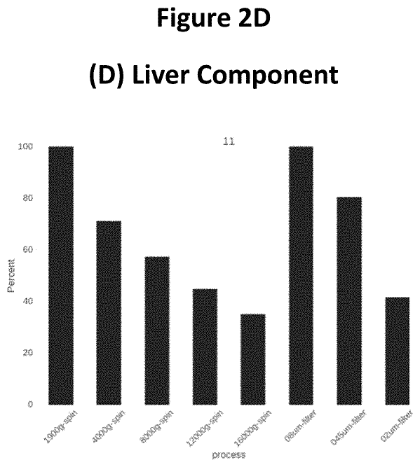

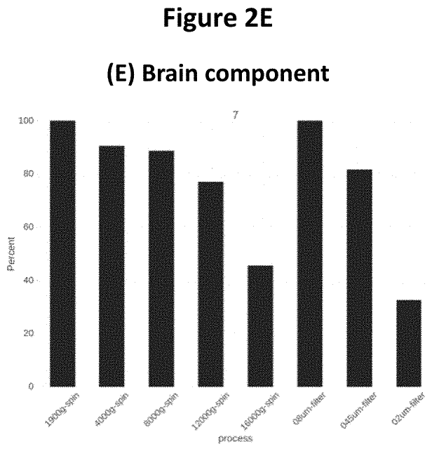

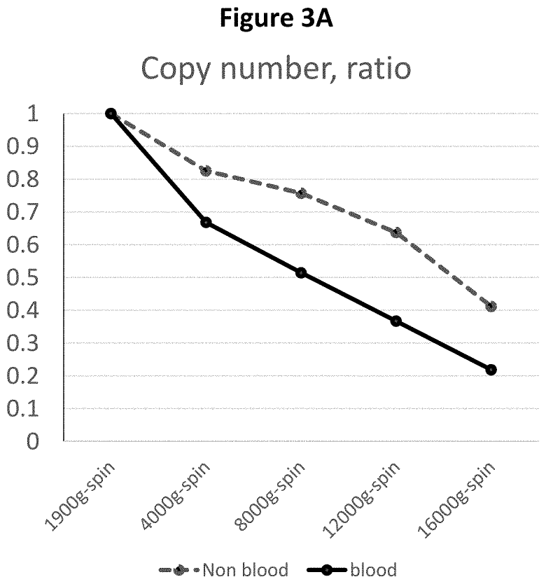

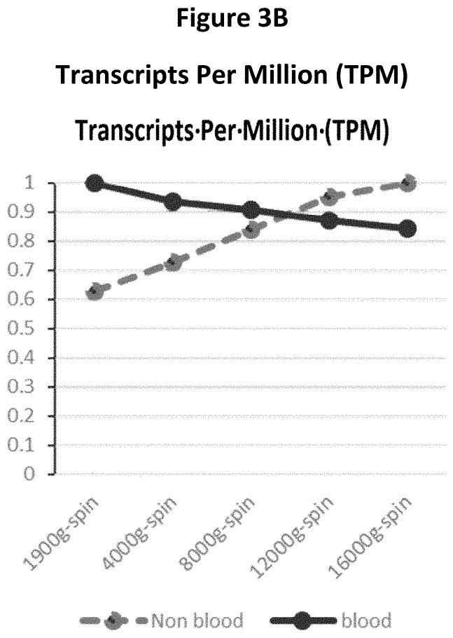

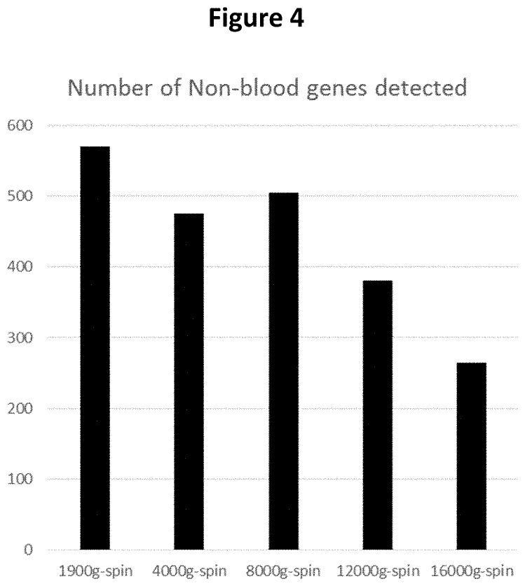

Size-selection performed on serum or plasma can increase the ratio of solid tissue-derived cf-mRNA compared to blood cell-derived cf-mRNA. To prepare serum or plasma, cells are sedimented in a 1,600 g spin. A second centrifugation step was implemented to enrich tissue-derived cf-mRNA. Plasma was centrifuged for 10 minutes at various speeds causing sedimentation forces ranging from 1,900 g to 16,000 g, followed by cf-RNA isolation, cDNA synthesis, library preparation, and sequencing. As the centrifugation speed increased, RNA transcript from blood cell components—platelet, and neutrophil transcripts (representative of transcripts from blood cells)—decreased more rapidly than tissue specific transcripts such as transcripts from liver or brain, resulting in an increase in the ratio of non-blood cf-mRNA to blood cell-derived cf-mRNA (FIGS. 2A-3B). This enrichment was counterbalanced by a decrease in the number of detectable tissue-derived genes (FIG. 4). The optimal speed for preparation of a low noise but representative and diverse cf-mRNA library depends upon the application and often ranges from 10,000 g to 16,000 g. For example, higher centrifugation speeds are preferred for the analysis of liver cf-mRNA transcripts compared to brain cf-mRNA transcripts. 16,000 g g was used for the results presented below.

Size selection was also performed by filtration through membranes with a size-cutoff of 0.8 μm, 0.45 μm, or 0.2 μm (FIG. 2). As the pore size decreased, RNA transcript from blood cell components—platelet, and neutrophil transcripts (representative of transcripts from blood cells)—decreased more rapidly than tissue specific transcripts such as transcripts from liver or brain, resulting in an increase in the ratio of non-blood cf-mRNA to blood cell-derived cf-mRNA. (FIG. 2).

### Example 3: Selection of Cf-mRNA Extraction Methodologies

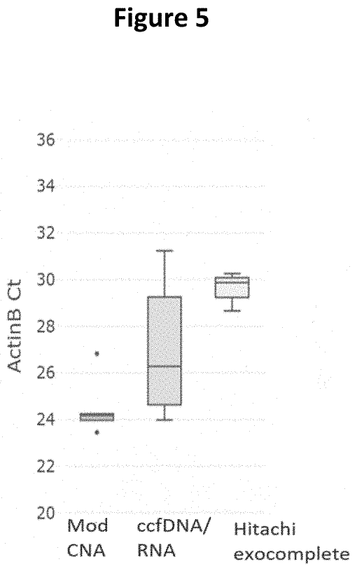

Various kits and methods were evaluated to optimize cf-mRNA extraction, including phenol based extractions of total cf-RNA: TRIzol, miRNeasy (Qiagen), Direct-zol (Zymo Research), nucleoZOL (Macherey-Nagel), mirVana (Life Technologies); extracellular vesicle capture based approaches followed by lysis (either phenol based or not): exoRNeasy (Qiagen), ExoComplete (Hitachi); immunoselection or immunodepletion of vesicles followed by extraction of nucleic acids; lysis followed by total RNA/nucleic acid isolation: Plasma/Serum RNA Purification Mini Kit (Norgen), QIAamp Circulating Nucleic Acids kit (“CNA kit”), QIAamp ccfDNA/RNA kit (Qiagen); and others. The CNA kit was selected because it showed the best balance between efficiency, scalability, linearity and consistency of cf-RNA extraction (see FIG. 5). The CNA kit is a total cf-RNA extraction kit which is agnostic to whether circulating cf-RNA is traveling as free RNA or is protected by proteins, lipids, or vesicles.

cf-RNA tends to be degraded in vivo and is further fragmented during the extraction process. miRNAs are also typically shorter than mRNAs. Thus Qiagen's “purification of miRNAs” protocol was selected instead of their standard “nucleic acid purification” protocol.

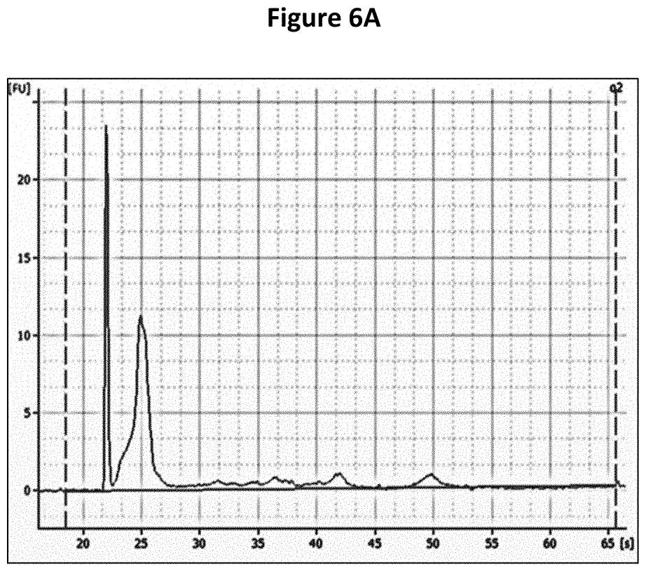

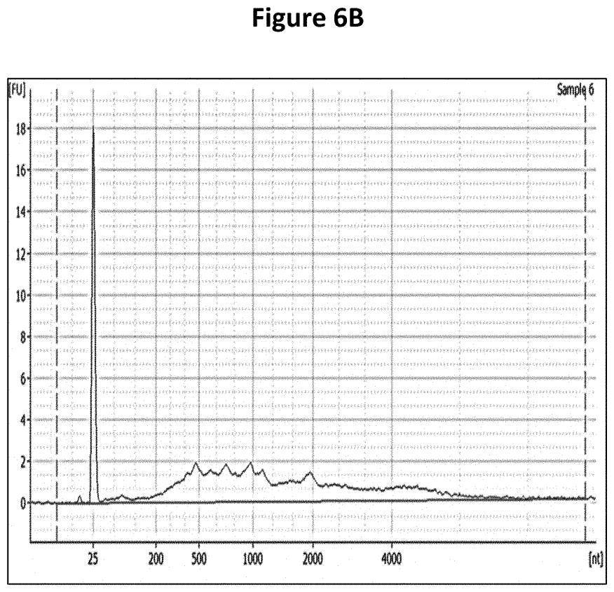

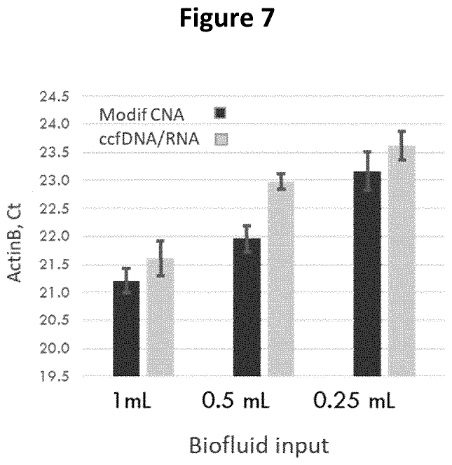

Multiple changes were made to the protocol supplied with the CNA kit to maximize the efficiency and the consistency of extraction. The protocol was adapted by (1) not adding carrier RNA to the lysis buffer because it interferes with sequencing results; (2) spiking in ERCC external reference RNAs during lysis; (3) pre-warming lysis buffers to lysis temperature (60° C. instead of 25° C.); (4) extending the lysis time from 30 minutes to a minimum of 45 minutes; (5) adding a second 100% ethanol washing step to better remove inhibitors from the sample; and (6) adding a second nucleic acid elution step to more completely remove RNA from the column. The size distribution of polynucleotides extracted with the modified method revealed an improved yield of fragmented cf-RNAs compared to the standard nucleic acid purification protocol (FIGS. 6A-6B). The modified extraction method with the CNA kit yielded more cf-mRNA than the QIAamp ccfDNA/RNA kit and showed better linearly with increased or reduced plasma input (FIG. 7).

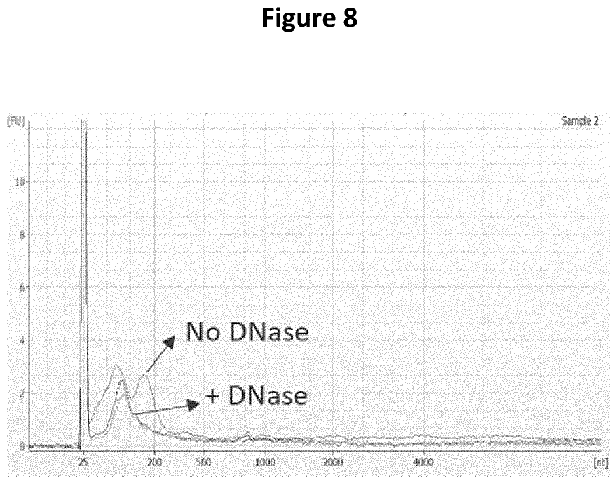

A dedicated enzymatic DNase step was incorporated into the protocol to remove DNA contamination and carry-over. Low level DNA contamination can be a source of error in gene-expression quantification and can be relevant for cf-RNA isolation because the amount of cf-RNA in serum or plasma is extremely low. Some commercially available cf-RNA extraction kits either ignore steps to remove DNA (e.g., many phenol-based kits) or recommend on-column DNase I treatments, which can be suboptimal for complete removal of DNA. Indeed, DNase I can be sensitive to salts, which are abundant during RNA extractions, and can have low efficiency with low DNA concentrations, which can be the case when working with cell-free biofluids. Turbo DNase (Ambion), a mutated version of DNase I with increased affinity for DNA, was utilized because it is particularly effective in removing trace quantities of DNA and more resistant to inhibitors. DNase treatment was performed according to the Ambion protocol except for an increase in the amount of enzyme to 2.5-3 μl enzyme per sample. DNase I treatment eliminated a substantial amount of contaminating nucleic acids that would otherwise interfere with cf-RNA analysis (FIG. 8).

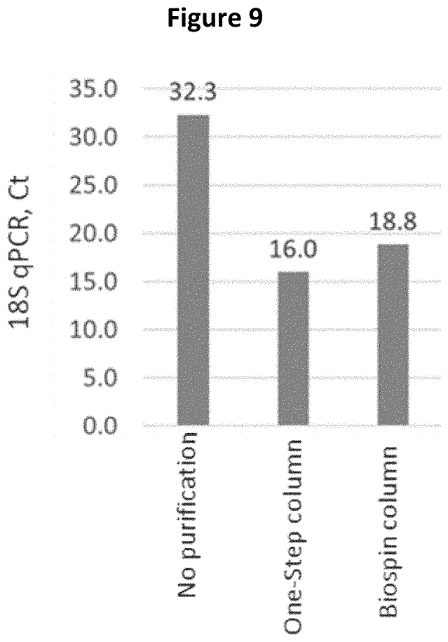

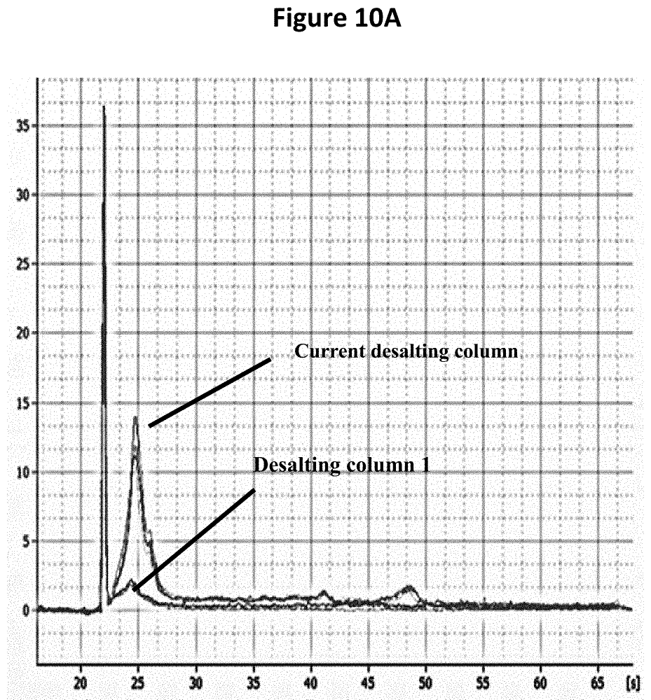

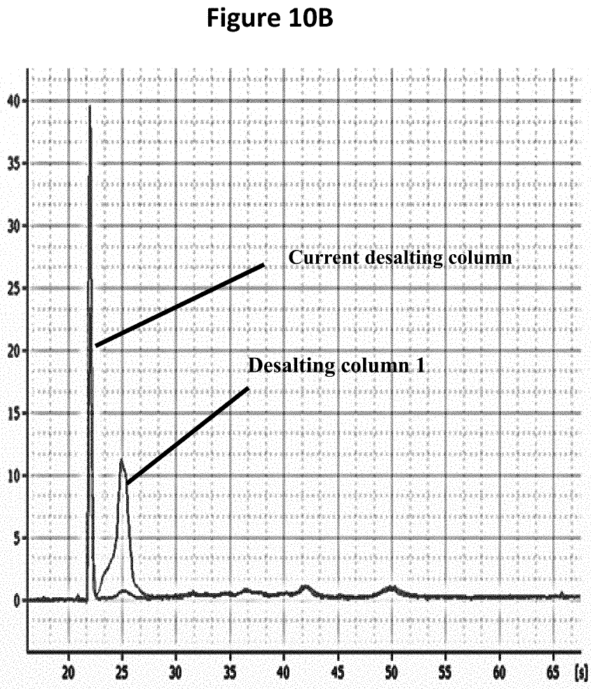

Titrating the input of extracted material into downstream reactions revealed variable traces of inhibitors from blood that lower the efficiency of biochemical reactions. Inhibitor removal and silica-membrane-based purification steps were therefore implemented after the DNase treatment. Inhibitor removal was performed with a OneStep PCR Inhibitor Removal kit (Zymo) with extra column washes to ensure complete removal of the prep buffer. Cleanup on this column increased the apparent yield of cf-RNA by removing contaminants that interfere with enzymes, and the OneStep column had a higher apparent yield than a Micro Bio-Spin column (Bio-Rad) (FIG. 9). Cleanup using the OneStep PCR Inhibitor Removal kit also preserved the recovery of fragmented cf-RNA polynucleotides (FIGS. 10A-10B). Silica-membrane purification was performed with a MinElute PCR Purification kit (Qiagen) using a larger volume of ethanol to maximize recovery.

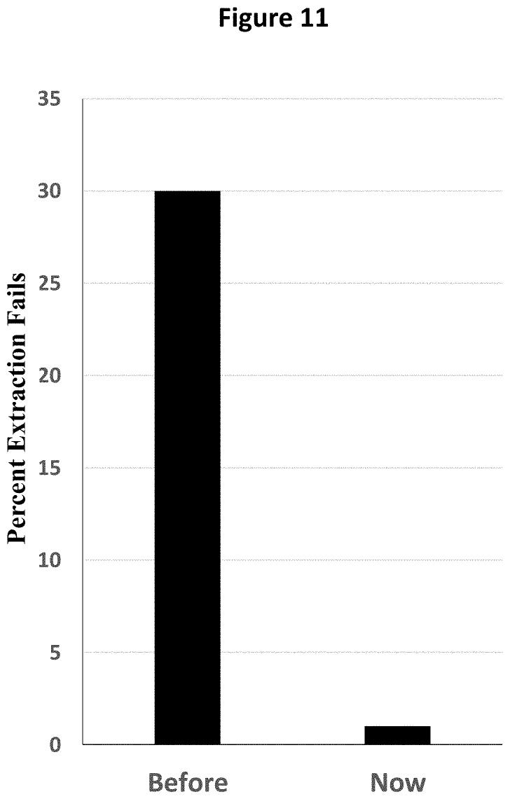

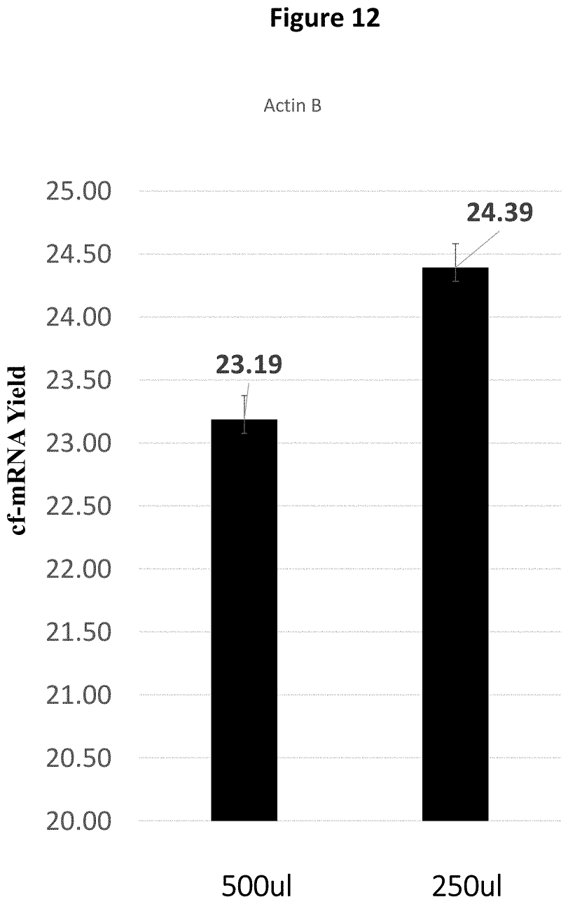

The final cf-RNA extraction and purification process dramatically reduced the frequency of assay fails due to suboptimal yields of less than 20 pg RNA and provided efficient and linear recovery of cell free mRNA using a range of input volumes (100ul to 3 ml) (FIGS. 11-12).

### Example 4: cDNA Synthesis, Library Preparation, and Whole Exome Capture

Commercially available low-input RNA sequencing kits generally include reagents for cDNA synthesis and removal of non-informative RNA species. The cDNA synthesis step in SMARTer (Takara) was found to be inefficient. A three-step strategy was therefore developed with a dedicated initial cDNA synthesis step, a second strand synthesis reaction, and a commercially available kit optimized for library preparation from low levels of dsDNA, followed by capture of the whole exome.

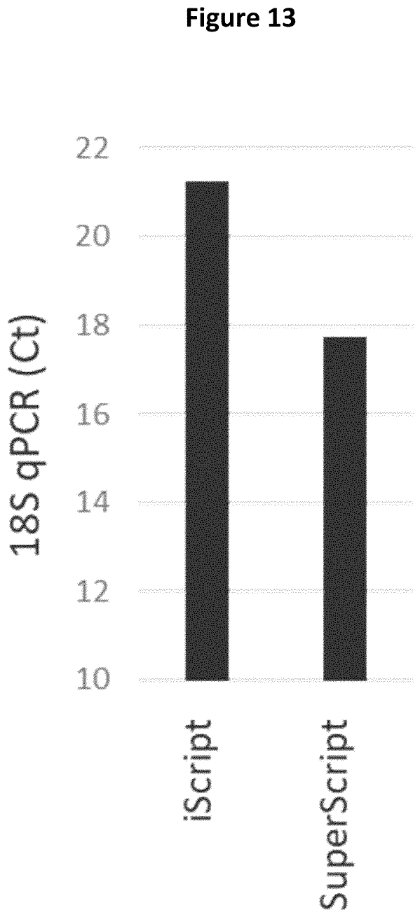

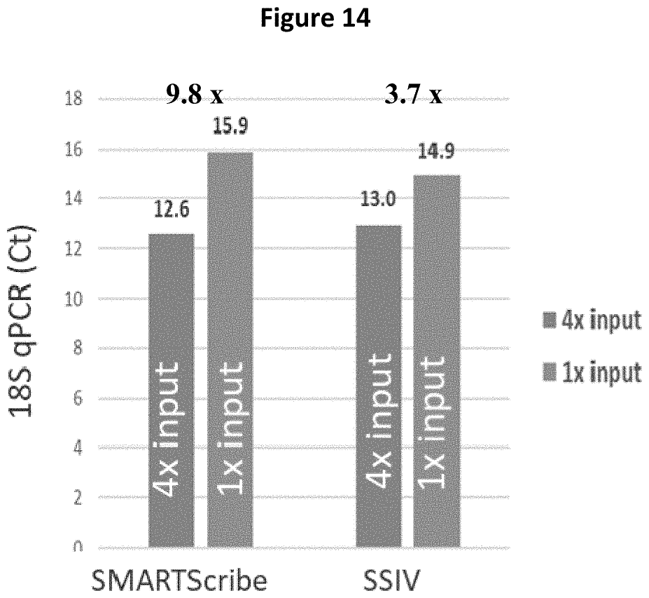

SuperScript IV (Invitrogen) was selected as the enzyme for reverse transcription because it demonstrated increased enzyme efficiency, linearity and resistance to traces of inhibitors with cf-RNA input when compared to iScript (Bio-Rad), qScript (Quantabio), SuperScript III (Invitrogen) and SMARTScribe (Takara). The conversion efficiency with SuperScript IV was further optimized by (1) priming with random hexamers instead of oligo dT, (2) increasing the primer concentration by 30-fold to 3 mg/ml, and (3) extending the reaction time from 10 minutes to 50 minutes as a precaution. The optimized SuperScript IV method yielded more cDNA than iScript (FIG. 13) and had better linearity than SMARTScribe (FIG. 14).

Double-stranded cDNA was generated in a second strand synthesis reaction using NEBNext Enzyme. No cleanup was performed between first and second strand synthesis. The second strand synthesis reaction was optimized by reducing the reagents used and total reaction volume by 50%.

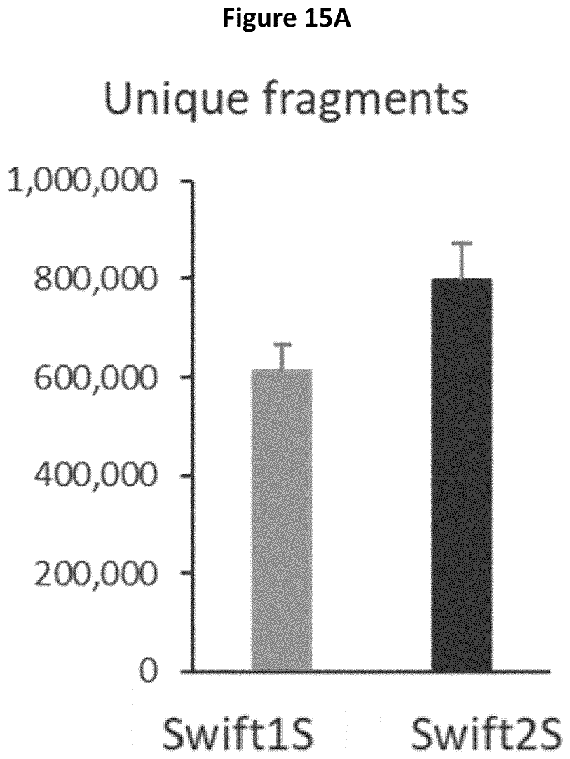

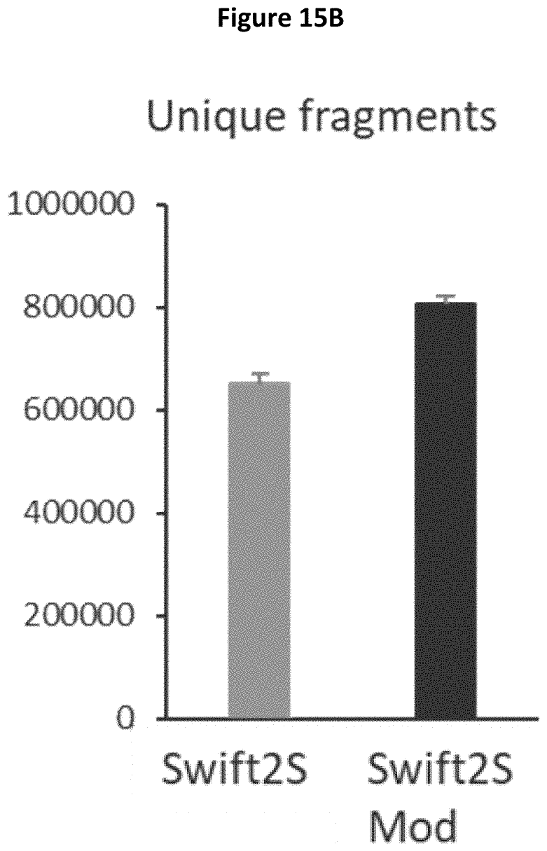

Accel-NGS 2S Plus was found to be the most robust and scalable library preparation method to generate sequencing libraries from low amounts of input cDNA when compared with Accel-NGS 1S Plus (Swift Biosciences) and others such as NxSeq® UltraLow DNA Library Preparation Kit (Lucigen). In particular, the number of unique sequence fragments was approximately 30% higher using Accel-NGS 2S Plus compared to Accel-NGS 1S Plus (FIG. 15A). Reagents remaining from the NEBNext second-strand synthesis step were not compatible with the chemistry of the Repair I step of Accel-NGS 2S Plus. The cDNA was therefore cleaned before Repair I using 1.8×SPRI beads. To minimize sample loss, Repair I was performed with the SPRI beads in the solution, and polyethylene glycol (PEG) was added after Repair I to promote DNA binding to the beads. To maximize recovery while avoiding contaminants including aptamers and dimers, the amounts of beads were adjusted to 1.8× for Repair I, 1.65 for Repair II, 1.65 for Ligation I and 1.4 for Ligation II. These modifications to the Accel-NGS 2S Plus protocol increased the number of unique sequence fragments by approximately 20% (FIG. 15B).

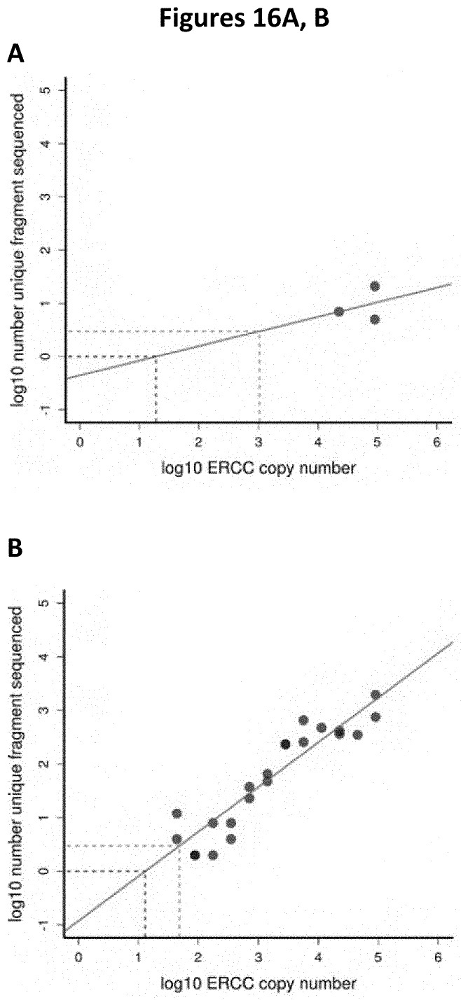

During PCR and before enrichment, each library prep was uniquely labeled with UDIs on both ends. UDIs were selected instead of standard indexes to minimize index hopping, which was especially important for cf-RNA libraries given the low number of copies of the input material. When standard indexes were used, sequencing reads were misassigned to a negative control (NTC). This contamination is alleviated by the use of UDIs (FIGS. 16A-16B).

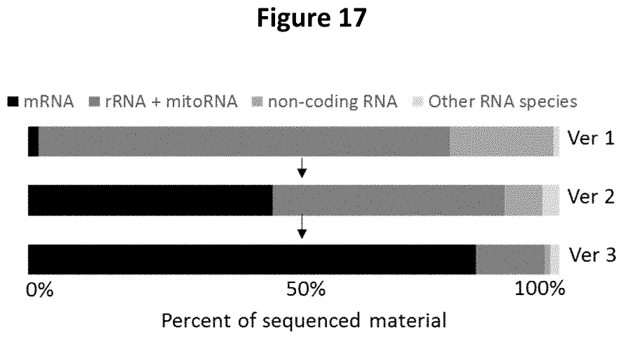

cf-mRNA was enriched from total cf-RNA by whole exome capture. This method was selected instead of rRNA depletion because mRNA constitutes less than 10% of the RNA molecules in circulation. Capture is performed with either RNA baits (Agilent) or DNA capture probes (IDT). RNA baits are preferred due to higher coverage of specific regions of interest. However, both can both be used. For normalization and quality control, the whole exome probes were combined with another set of probes designed to capture the 35 ERCC standards covering a wide range of copy numbers and sizes that were spiked during the extraction step. Capture was performed on pools of up to 10 cDNA samples according to a modified version of an Agilent protocol using XT2 blockers and reagents with XT probes. The percentage of RNA polynucleotides arising from mRNA and other sources such as ribosomal RNA, mitochondrial RNA, non-coding RNA and other RNA species was determined by sequencing to compare different enrichment strategies. The mRNA fraction was substantially higher with whole exome capture compared to negative enrichment by rRNA depletion and the starting pool of total RNA (FIG. 17). In particular, approximately 80% of the sequence reads from the whole exon capture material were mRNA sequences, approximately 45% of the sequences after rRNA depletion were mRNA sequences, and less than 5% of sequences from total RNA were mRNA sequences.

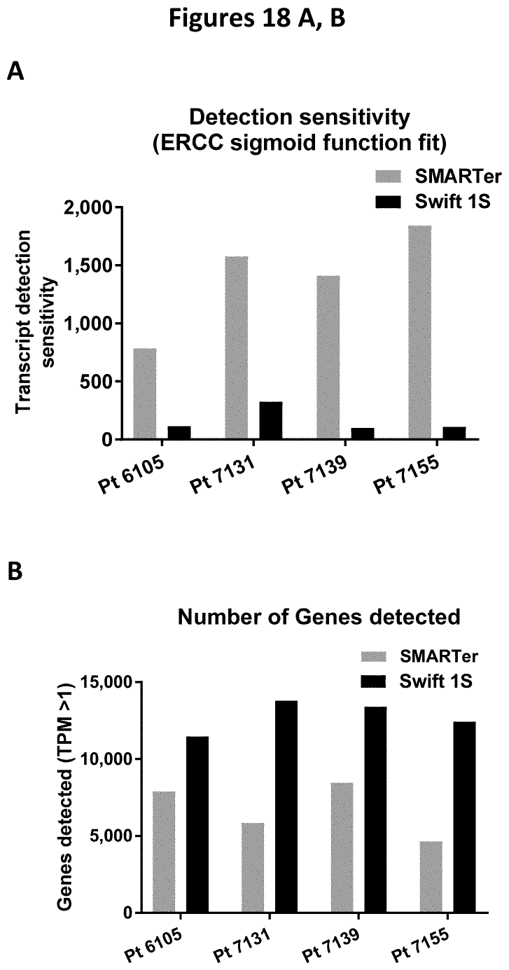

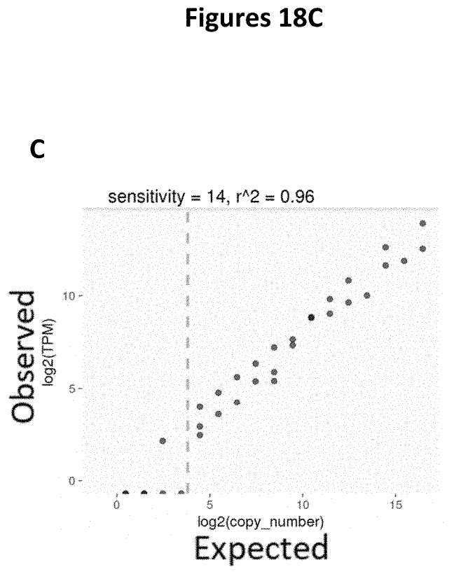

The cDNA synthesis, library preparation, and whole exome capture methods presented in this section yielded sequencing reads more representative of the broad spectrum of cf-mRNAs compared to libraries constructed and depleted of rRNA using the SMARTer kit. The sensitivity for detecting low amounts of spiked in ERCC standards was improved by a factor of 5 to 20 (FIG. 18A). This increased sensitivity facilitated detection of substantially more protein coding genes (FIG. 18B). Using spiked in ERCC standards of known concentration, the sensitivity of detection was estimated to be approximately 14 copies (FIG. 18C).

### Example 5: Comparison to Other Cf-mRNA Libraries

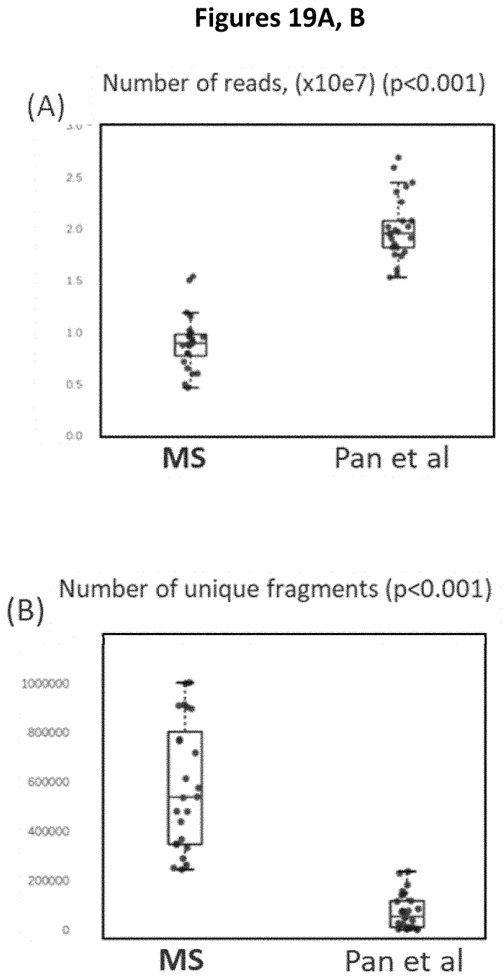

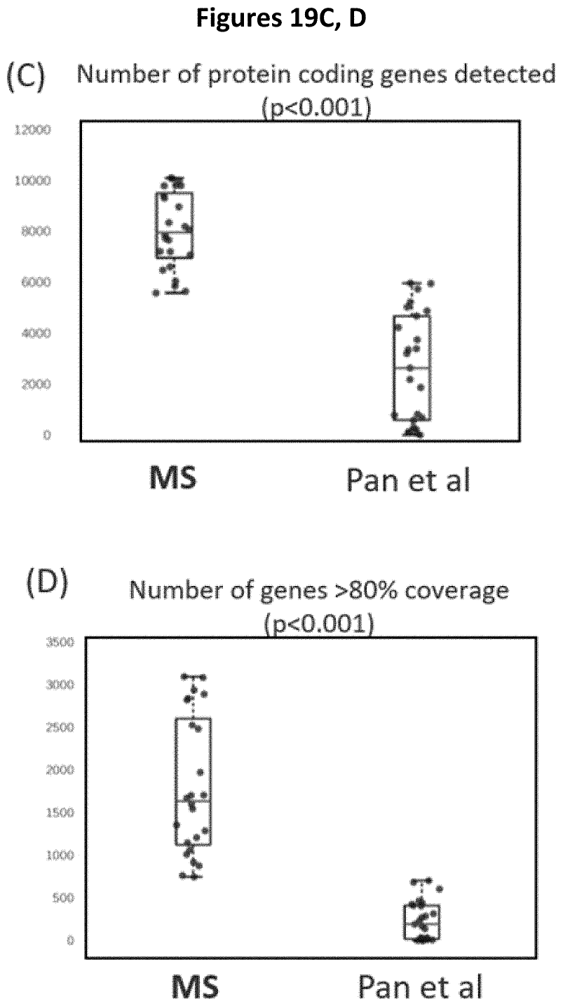

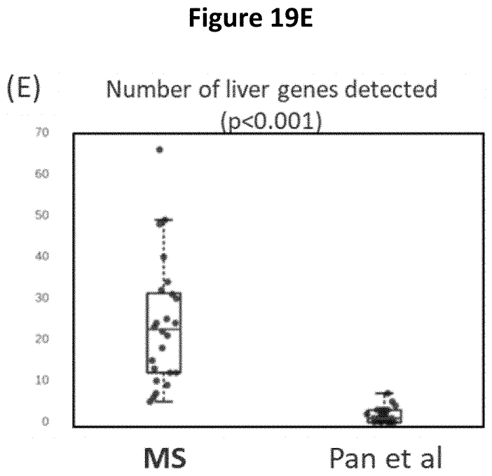

Cell-free-mRNA libraries prepared by the method of Example 1 were superior to the “Pan et al” cell-free mRNA libraries (Pan et al, Clin Chem. 2017 November; 63(11):1695-1704). For this analysis, raw sequencing data from both studies was processed using the bioinformatics pipeline described in Example 1. The Pan libraries were prepared from the equivalent of 500 μl of serum per prep, whereas only 165 μl of serum per prep was used to prepare the Example 1 libraries. Despite approximately two-fold fewer sequencing reads per prep (FIG. 19A), the Example 1 protocol (modified with centrifugation at 16,000 g and enrichment with DNA capture probes) yielded approximately six-fold more unique fragments (FIG. 19B), approximately three-fold more protein coding genes (FIG. 19C), approximately four-fold more genes with >80% coverage (FIG. 19D), and approximately eight-fold more liver genes (FIG. 19E).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of preparing a cf-RNA sample, the method comprising:
(a) centrifuging a biological sample at from 1,600 g to 16,000 g; and
(b) isolating RNA from the biological sample;
wherein at least 500, 600, 700, 800, 900, 1000, 1100, 1200, 1300, 1400, or 1500 non-blood genes selected from the list in Table 1 or low stringency non-blood genes selected from Table 10 are present in the cf-RNA sample.

2.-86. (canceled)
